(19) United States
(12) Patent Application Publication
(10) Pub. No.: US 2023/0192660 A1
(43) Pub. Date: Jun. 22, 2023

(54) Protease Inhibitors for Treatment or Prevention of Coronavirus Disease

(71) Applicant: The Board of Trustees of the Leland Stanford Junior University, Stanford, CA (US)

(72) Inventors: Michael Z. Lin, Stanford, CA (US); Xinzhi Zou, Stanford, CA (US); Michael Westberg Soerensen, Stanford, CA (US); Lin Ning, Redwood City, CA (US); Yichi Su, Berkeley, CA (US)

(21) Appl. No.: 17/922,555

(22) PCT Filed: May 7, 2021
(86) PCT No.: PCT/US21/31425

§ 371 Date: Oct. 31, 2022

Related U.S. Application Data
(60) Provisional application No. 63/021,863, filed on May 8, 2020.
(60) Provisional application No. 63/052,153, filed on Jul. 15, 2020.
(60) Provisional application No. 63/078,491, filed on Sep. 15, 2020.

(51) Int. Cl. C07D 403/12; C07D 401/12; C07D 403/14; A61K 31/40; A61K 31/497; A61K 31/422; A61K 31/706; G01N 33/58; A61P 31/14

(52) U.S. Cl. CPC C07D 403/12; C07D 401/12; C07D 403/14; A61K 31/40; A61K 31/497; A61K 31/422; A61K 31/706; G01N 33/582; A61P 31/14

(57) ABSTRACT

Provided are protease inhibitor compounds that find use in treating or preventing coronavirus disease. In some embodiments, the coronavirus disease is COVID-19. Also provided are compositions and kits comprising the compounds, as well methods of using the compounds to treat or prevent coronavirus disease. Methods of assessing inhibition of coronavirus protease activity by an agent are also provided.

### CROSS-REFERENCE TO RELATED APPLICATIONS

This application claims the benefit of U.S. Provisional Patent Application No. 63/021,863, filed May 8, 2020, U.S. Provisional Patent Application No. 63/052,153, filed Jul. 15, 2020, and U.S. Provisional Patent Application No. 63/078,491, filed Sep. 15, 2020, which applications are incorporated herein by reference in their entireties.

### INTRODUCTION

A novel coronavirus, Severe Acute Respiratory Syndrome Coronavirus 2 (SARS-CoV-2), was first identified in December 2019 as the cause of a respiratory illness designated coronavirus disease 2019, or COVID-19. A new clinical syndrome, COVID-19 is characterized by respiratory symptoms with varying degrees of severity, from mild upper respiratory illness to severe interstitial pneumonia and acute respiratory distress syndrome, aggravated by thrombosis in the pulmonary microcirculation. Its clinical evolution is characterized by three main phases—early infection phase, pulmonary phase, and hyperinflammation phase—with clinical features ranging from mild or no symptoms to acute respiratory distress syndrome and multi-organ failure.

SARS-CoV-2 is a positive-sense single-stranded RNA virus that belongs to the (3-coronaviruse family along with SARS and MERS. The SARS-CoV-2 genome contains five genes that code for four structural proteins—spike (S), envelope (E), membrane (M) and nucleocapsid (N)—and 16 non-structural proteins. Viral entry into human cells is mediated by an interaction between the S glycoprotein and the Angiotensin-Converting Enzyme 2 (ACE2) receptor. ACE2 is a metalloprotease that lowers blood pressure by catalyzing the hydrolyses of angiotensin II. ACE2 enzymatic activity is not related, or needed, in SARS-CoV-2 entry into the host cells.

A number of investigational agents and drugs that are approved for other indications are currently being evaluated in clinical trials for the treatment of COVID-19 and associated complications. Data from randomized controlled trials, prospective and retrospective observational cohorts, and case series studies are rapidly emerging. Remdesivir (GS-5734), an inhibitor of the viral RNA-dependent, RNA polymerase with in vitro inhibitory activity against SARS-CoV-1 and the Middle East respiratory syndrome (MERS-CoV), was identified early as a promising therapeutic candidate for COVID-19 because of its ability to inhibit SARS-CoV-2 in vitro. The U.S. Food and Drug Administration (FDA) recently approved remdesivir for the treatment of patients with COVID-19 requiring hospitalization. However, a study of more than 11,000 people in 30 countries sponsored by the World Health Organization found that remdesivir had little or no effect on hospitalized COVID-19, as indicated by overall mortality, initiation of ventilation and duration of hospital stay. As such, there remains a need for effective therapeutics for treatment of SARS-CoV-2 infection and COVID-19.

### SUMMARY

Provided are protease inhibitor compounds that find use in treating or preventing coronavirus disease. In some embodiments, the coronavirus disease is COVID-19. Also provided are compositions and kits comprising the compounds, as well methods of using the compounds to treat or prevent coronavirus disease. Methods of assessing inhibition of coronavirus protease activity by an agent are also provided.

### BRIEF DESCRIPTION OF THE FIGURES

FIG. 1: Models of boceprevir, telaprevir, narlaprevir and rupintrivir in the active site of SARS-CoV-2.

FIG. 2: Schematic diagram of a fusion protein and method for assessing inhibition of coronavirus protease activity by an agent.

FIG. 3: Fluorescence imaging data for various compounds employed in the method schematically illustrated in FIG. 2.

FIG. 4: Cellular localization/substrate cleavage data shown by immunoblot for various compounds employed in the method schematically illustrated in FIG. 2.

FIGS. 5-6: Compounds according to some embodiments of the present disclosure.

FIG. 7: Preliminary data demonstrating the potency of inhibition of SARS-CoV-2 MPro by compounds according to some embodiments of the present disclosure.

FIGS. 8-14: Compounds according to some embodiments of the present disclosure.

FIG. 15: Cocrystal structure of SARS-CoV-2 Mpro and ML1000.

FIG. 16: Cocrystal structure of SARS-CoV-2 Mpro and ML1001.

FIG. 17: Cocrystal structure of SARS-CoV-2 Mpro and ML102.

FIG. 18: Cocrystal structure of SARS-CoV-2 Mpro and ML104.

FIG. 19: Schematic illustration of a reporter polypeptide (top) and a method of using the reporter polypeptide in an assay for protease inhibition.

FIG. 20: Inhibition data from the assay schematically illustrated in FIG. 19 for compounds according to some embodiments of the present disclosure.

FIG. 21: Data demonstrating inhibition of viral replication in A549-ACE2 cells by compounds according to some embodiments of the present disclosure.

FIG. 22: HCV protease inhibitors with a P2 proline analog can be docked into the SARS-CoV-2 M^{pro} active site. (A) Co-crystal structure of SARS-CoV-2 M^{pro} and inhibitor 13b (PDB 6Y2G). (B) Using Pymol, boceprevir was placed into the SARSCoV2 M^{pro} active site and unconstrained bonds were manually rotated for optimal complementary with the S1 and S2 pockets and hydrogen-bonding to the backbone carbonyl of Glu-166. (C) Telaprevir was similarly docked into the SARS-CoV-2 M^{pro} active site for optimal complementary with the S1, S2, and S4 pockets and hydrogen-bonding to the backbone carbonyl of Glu-166. (D) Alignment of the 13b-M^{pro} cocrystal with the manually docked boceprevir structure shows that the backbone of the P2-analogous segment of 13b is superimposable with the proline analog of boceprevir.

FIG. 23: Inhibition of SARS-CoV-2 M^{pro} activity in vitro. (A) Left, relative M^{pro} activity in the presence of 2 mM DTT and 150 μM of drugs. GC-376 showed the most inhibition, followed by boceprevir, then telaprevir and narlaprevir. The HIV protease inhibitor ritonavir, ebselen, and disulfiram showed less than 50% inhibiting of enzyme activity, indicating IC₅₀>150 μM in reducing conditions. Right, in the absence of DTT, ebselen and disulfiram showed efficient inhibition of M^{pro} activity. Enzymatic assays were carried out with 100 nM purified SARS-CoV-2 M^{pro} with an uncleaved C-terminal His₆-tag, M^{pro}-His₆(B) IC₅₀ measurements by inhibitor titrations on 100 nM SARS-CoV-2 M^{pro}-His₆ (top) or 100 nM fully mature SARSCoV2 M^{pro} (bottom). For ebselen and disulfram, measurements were performed in the absence of DTT, while the assay buffer contained 2 mM DTT for all other drugs. Mean values of 2 to 3 independent experiments are shown. Error bars represent standard deviation.

FIG. 24: Design and in vitro potency of a novel coronavirus M^{pro} inhibitors. (A) Structures of boceprevir, telaprevir, ML1000, and ML1100. ML1000 is essentially boceprevir with a γ-lactamyl group in place of the P1 cyclobutanyl group. ML1100 replaces the bicyclic P2 proline analog of boceprevir with that of telaprevir. (B) IC₅₀ measurements of ML1000 and ML1100 with 100 nM mature SARSCoV2 M^{pro}. With enzyme concentration at 100 nM, the lowest possible IC₅₀ that can be detected in theory is 50 nM. Thus, IC₅₀ values were also measured for ML1000 and GC-376 with 20 nM enzyme. (C) IC₅₀ values of ML1000 and GC-376 measured with 20 nM mature SARS-CoV-2 M^{pro} were 12 and 14 nM, respectively. Thus, ML1000 and GC-376 shows tight binding, even at 20 nM of M^{pro}, and the IC₅₀ values may still be limited by the enzyme concentration. Mean values of 3 independent experiments are shown. Error bars represent standard deviation.

FIG. 25: Potency of ML1000 and ML1100 in inhibiting M^{pro} activity in Huh7 cells. Above, representative immunoblot of lysates of Huh7 cells transduced with lentivirus encoding the construct shown in FIG. 2A. Immunoblots were probed with antibodies against FLAG to detect the substrate protein and beta-actin as a control. Below, the fraction of cleaved substrate relative to the total substrate abundance was quantified at different inhibitor concentrations and normalized to the cleavage ratio in absence of inhibitor. Mean values of 3 independent experiments are shown. Error bars represent standard deviation. The relative potency of the drugs in this assay is GC-376>ML1000>ML1100>boceprevir.

FIG. 26: Inhibition of SARS-CoV-2 replication in Caco-2 cells. The viral titer was quantified after incubation with different concentrations of inhibitor. For each condition, three technical replicates were combined for a single measurement. The data were plotted on a log-log graph and fit to a logistic function. EC₅₀ values were calculated as the inhibitor concentration needed to reduce the viral titer by one log 2 unit relative to the upper baseline, and the corresponding points on the curves are indicated with squares on each fitted curve.

### DEFINITIONS

The following terms have the following meanings unless otherwise indicated. Any undefined terms have their art recognized meanings.

“Alkyl” refers to monovalent saturated aliphatic hydrocarbyl groups having from 1 to 10 carbon atoms and such as 1 to 6 carbon atoms, or 1 to 5, or 1 to 4, or 1 to 3 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

The term “haloalkyl” refers to a substituted alkyl group as described above, wherein one or more hydrogen atoms on the alkyl group have been substituted with a halo group. Examples of such groups include, without limitation, fluoroalkyl groups, such as trifluoromethyl, difluoromethyl, trifluoroethyl and the like.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

The term “substituted alkynyl” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, and —SO₂-heteroaryl.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

“Acylamino” refers to the groups —NR²⁰C(O)alkyl, —NR²⁰C(O)substituted alkyl, NR²⁰C(O)cycloalkyl, —NR²⁰C(O)substituted cycloalkyl, NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂— substituted alkyl, —SO₂-aryl, —SO₂-heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

“Amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group —N₃.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl and —SO₂-heteroaryl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic and at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

“Heteroarylalkyl” by itself or as part of another substituent, refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. In certain embodiments, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl. In certain embodiments, the heteroarylalkyl group is 6-20 membered heteroarylalkyl, e.g., the alkanyl, alkenyl or alkynyl moiety of the heteroarylalkyl is 1-8 membered and the heteroaryl moiety is a 5-12-membered heteroaryl.

The term “heteroaralkyl” refers to the groups -alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Heteroalkyl, Heteroalkanyl, Heteroalkenyl and Heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups which can be included in these groups include, but are not limited to, —O—, —S—, —S—S—, —O—S—, —NR³⁷R³⁸—, ·═N—N═, —N═N—, —N═N—NR³⁹R⁴⁰, —PR⁴¹—, —P(O)₂—, —POR⁴²—, —O—P(O)₂—, —S—O—, —S—(O)—, —SO₂—, —SnR⁴³R⁴⁴— and the like, where R³⁷, R³⁸, R³⁹, R⁴⁰, R⁴¹, R⁴², R⁴³ and R⁴⁴ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻ M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of 0, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as +N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃-M⁺, —OSO₃R⁷⁰, —PO₃-2(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or -S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in certain embodiments, a group that is substituted has 1, 2, 3, or 4 substituents, 1, 2, or 3 substituents, 1 or 2 substituents, or 1 substituent.

### DETAILED DESCRIPTION

Before the compounds, compositions and methods of the present disclosure are described in greater detail, it is to be understood that the compounds, compositions and methods are not limited to particular embodiments described, as such may, of course, vary. It is also to be understood that the terminology used herein is for the purpose of describing particular embodiments only, and is not intended to be limiting, since the scope of the compounds, compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the compounds, compositions and methods. The upper and lower limits of these smaller ranges may independently be included in the smaller ranges and are also encompassed within the compounds, compositions and methods, subject to any specifically excluded limit in the stated range. Where the stated range includes one or both of the limits, ranges excluding either or both of those included limits are also included in the compounds, compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the compounds, compositions and methods belong. Although any compounds, compositions and methods similar or equivalent to those described herein can also be used in the practice or testing of the compounds, compositions and methods, representative illustrative compounds, compositions and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present compounds, compositions and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the compounds, compositions and methods, which are, for clarity, described in the context of separate embodiments, may also be provided in combination in a single embodiment. Conversely, various features of the compounds, compositions and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present compounds, compositions and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Compounds

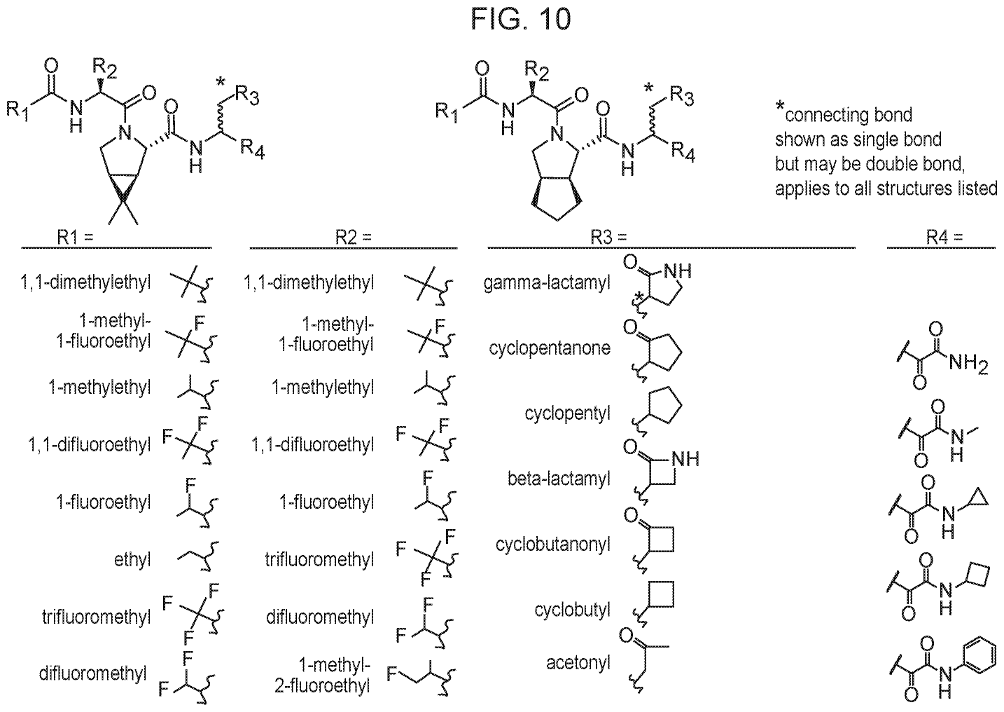

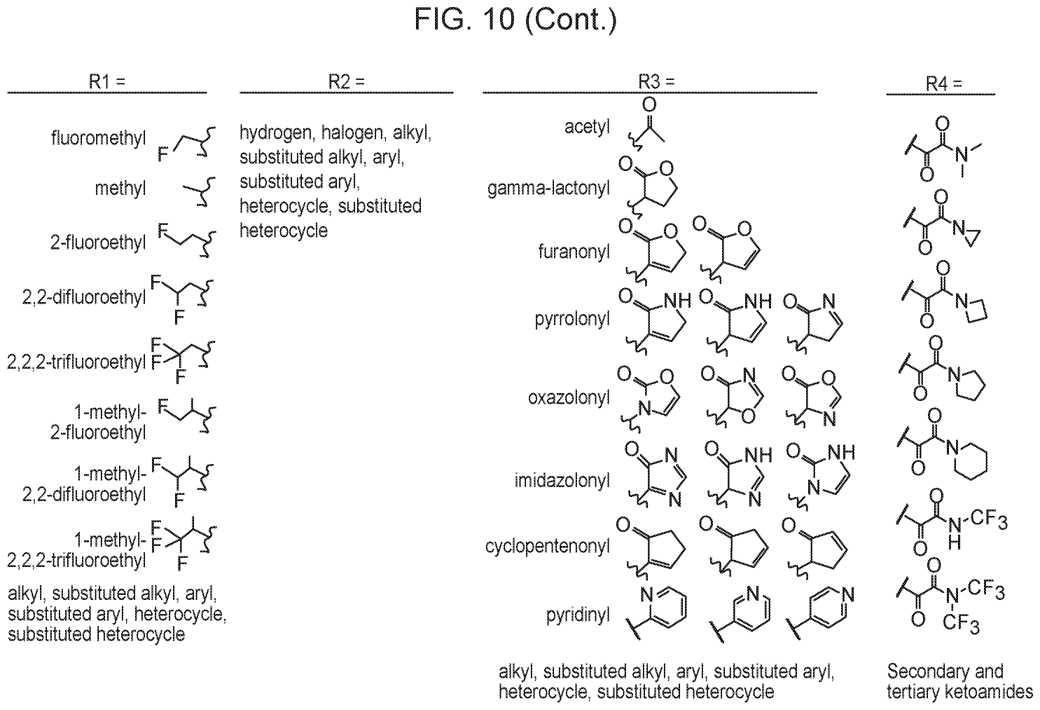

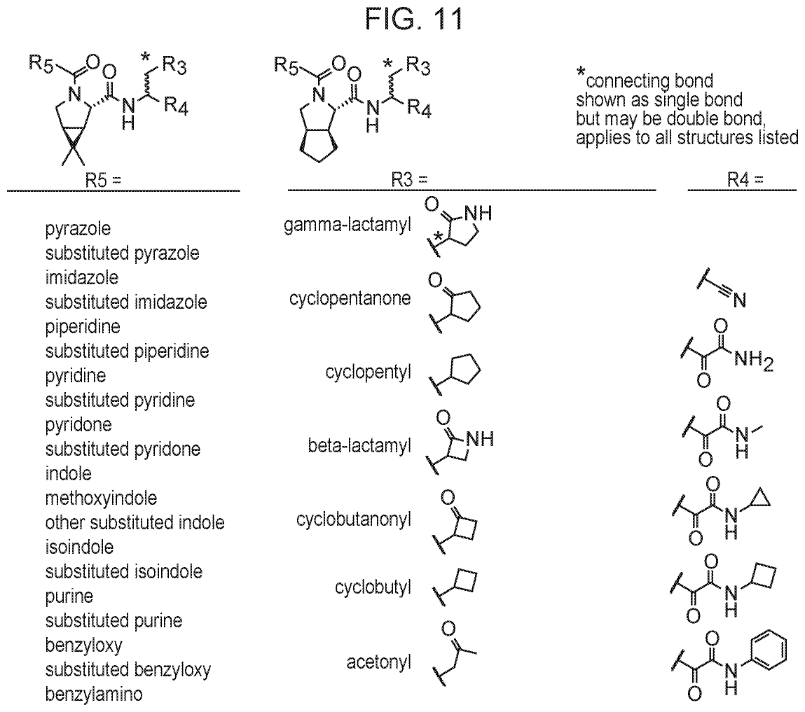

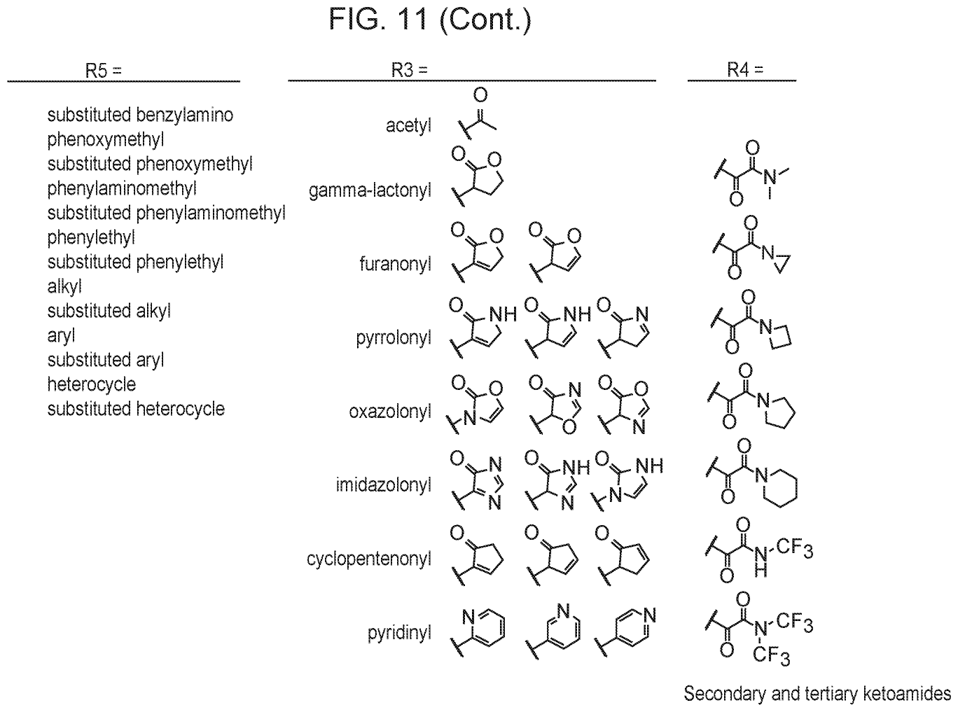

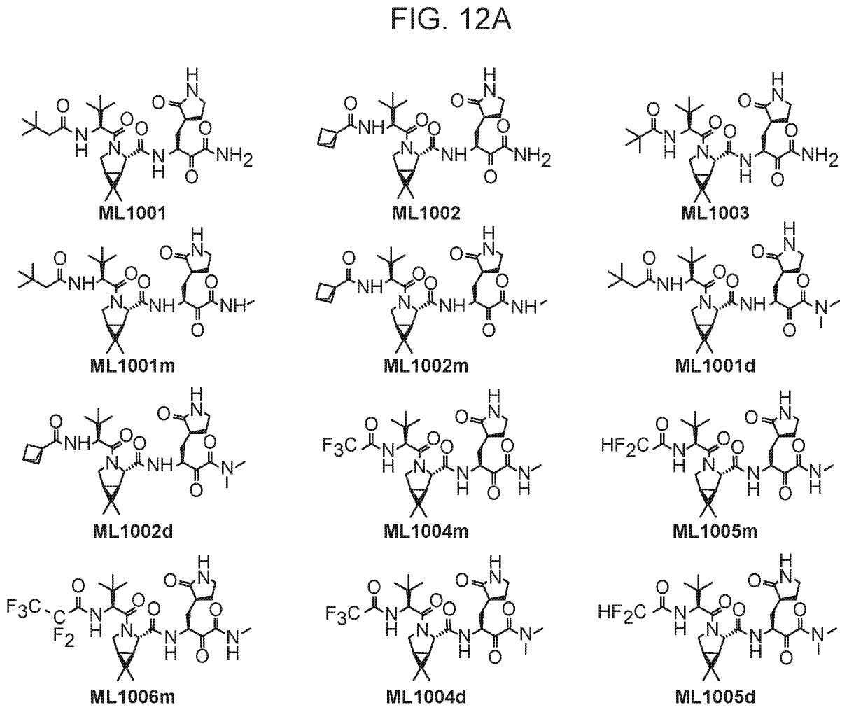

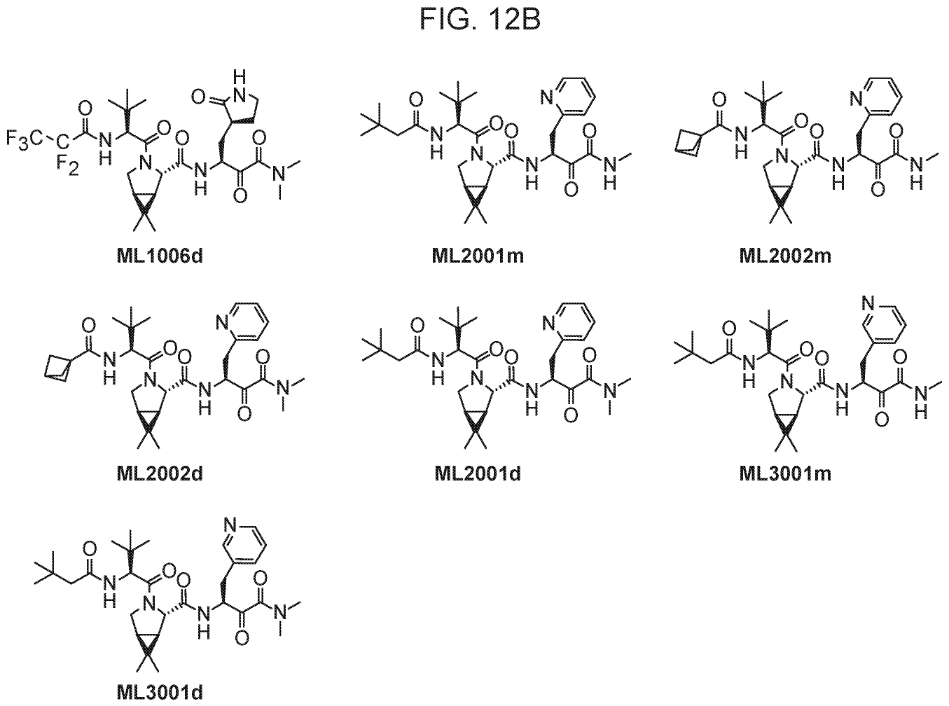

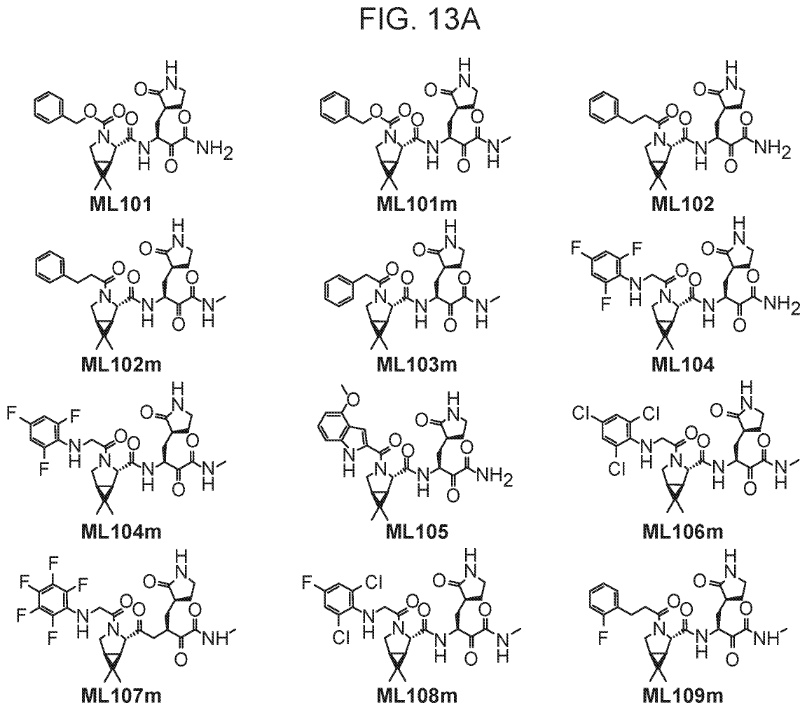

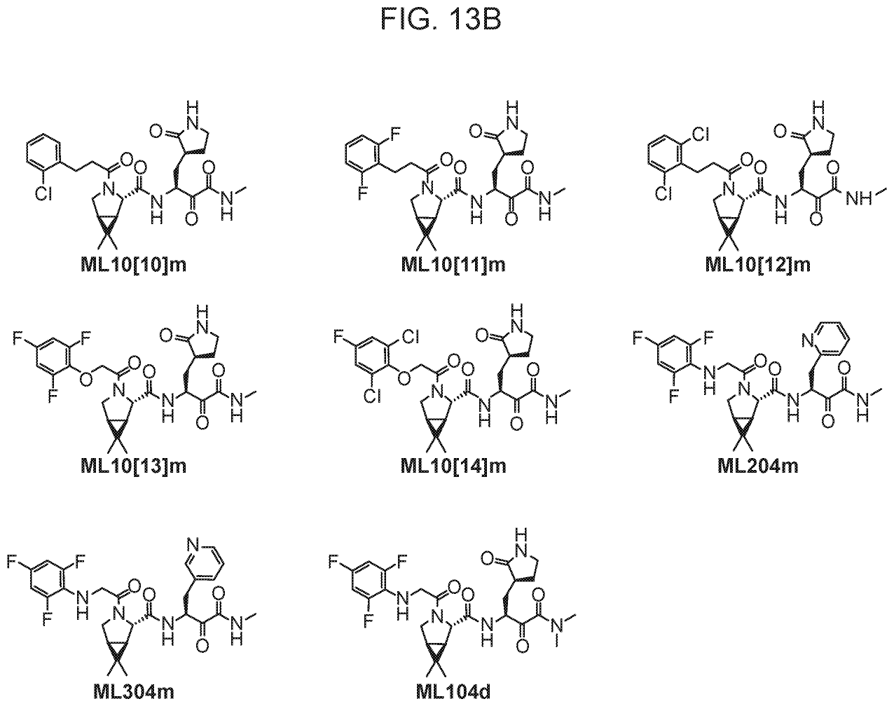

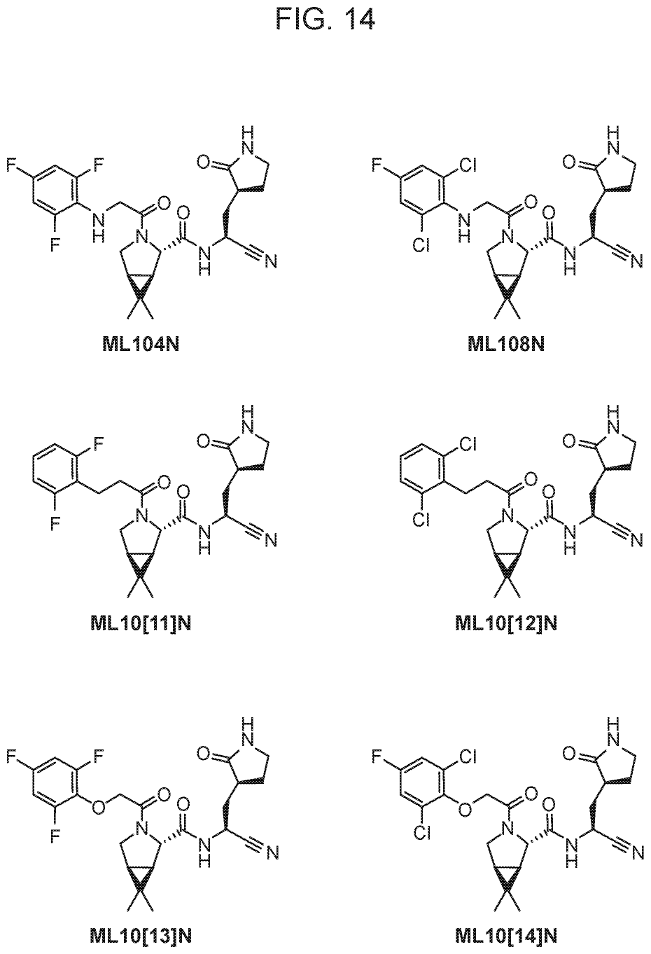

Provided by the present disclosure are ketoamide and other reversible covalent protease inhibitor compounds that find use in inhibiting viral protease activity (e.g., coronavirus viral protease activity, such as SARS-CoV-2 protease activity). In turn, the compounds find use in inhibiting viral replication for, e.g., treating and/or preventing a coronavirus infection in an individual in need thereof. In certain embodiments, the compounds of the present disclosure are alkylated ketoamide-based or nitrile-based protease inhibitor compounds, such as any of the compounds shown in FIGS. 5, 6 and 8-14 herein. Details regarding these and other compounds are provided below.

According to some embodiments, a compound of the present disclosure has the formula:

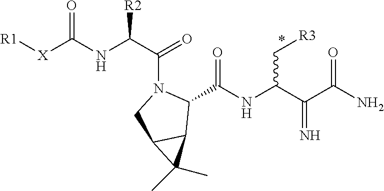

wherein:
R1 is 1,1-dimethylethyl, 1-methyl-1-fluoroethyl, 1-methylethyl, 1,1-difluoroethyl, 1-fluoroethyl, ethyl, trifluoromethyl, difluoromethyl, fluoromethyl, methyl, 2-fluoroethyl, 2,2-difluoroethyl, 2,2,2-trifluoroethyl, 1-methyl-2-fluoroethyl, 1-methyl-2,2-difluoroethyl, 1-methyl-2,2,2-trifluoroethyl, alkyl, substituted alkyl, aryl, substituted aryl, heterocycle, or substituted heterocycle;
R2 is 1,1-dimethylethyl, 1-methyl-1-fluoroethyl, 1-methylethyl, 1,1-difluoroethyl, 1-fluoroethyl, trifluoromethyl, difluoromethyl, 1-methyl-2-fluoroethyl, hydrogen, halogen, alkyl, substituted alkyl, aryl, substituted aryl, heterocycle, or substituted heterocycle;
R3 is gamma-lactamyl, cyclopentanonyl, cyclopentyl, beta-lactamyl, cyclobutanonyl, cyclobutyl, acetonyl, acetyl, gamma-lactonyl, furanonyl, pyrrolonyl, cyclopentenonyl, oxazolonyl, imidazolonyl, alkyl, substituted alkyl, aryl, substituted aryl, heterocycle, or substituted heterocycle;
X is CH₂, NH, or O; and
the bond indicated by the asterisk is a single or double bond.

In certain embodiments, such as compound is:

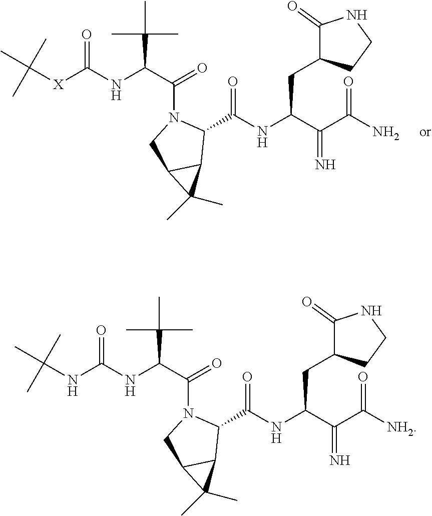

According to some embodiments, a compound of the present disclosure has the formula:

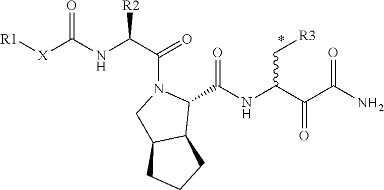

wherein:
R1 is 1,1-dimethylethyl, 1-methyl-1-fluoroethyl, 1-methylethyl, 1,1-difluoroethyl, 1-fluoroethyl, ethyl, trifluoromethyl, difluoromethyl, fluoromethyl, methyl, 2-fluoroethyl, 2,2-difluoroethyl, 2,2,2-trifluoroethyl, 1-methyl-2-fluoroethyl, 1-methyl-2,2-difluoroethyl, 1-methyl-2,2,2-trifluoroethyl, alkyl, substituted alkyl, aryl, substituted aryl, heterocycle, or substituted heterocycle;
R2 is 1,1-dimethylethyl, 1-methyl-1-fluoroethyl, 1-methylethyl, 1,1-difluoroethyl, 1-fluoroethyl, trifluoromethyl, difluoromethyl, 1-methyl-2-fluoroethyl, hydrogen, halogen, alkyl, substituted alkyl, aryl, substituted aryl, heterocycle, or substituted heterocycle;
R3 is gamma-lactamyl, cyclopentanonyl, cyclopentyl, beta-lactamyl, cyclobutanonyl, cyclobutyl, acetonyl, acetyl, gamma-lactonyl, furanonyl, pyrrolonyl, cyclopentenonyl, oxazolonyl, imidazolonyl, alkyl, substituted alkyl, aryl, substituted aryl, heterocycle, or substituted heterocycle;
X is CH₂, NH, or O; and
the bond indicated by the asterisk is a single or double bond.

In certain embodiments, such as compound is:

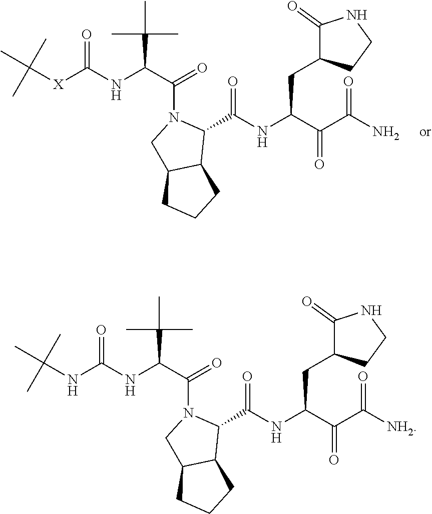

According to some embodiments, a compound of the present disclosure has the formula:

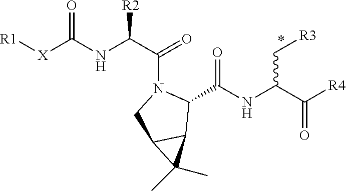

wherein:
R1 is 1,1-dimethylethyl, 1-methyl-1-fluoroethyl, 1-methylethyl, 1,1-difluoroethyl, 1-fluoroethyl, ethyl, trifluoromethyl, difluoromethyl, fluoromethyl, methyl, 2-fluoroethyl, 2,2-difluoroethyl, 2,2,2-trifluoroethyl, 1-methyl-2-fluoroethyl, 1-methyl-2,2-difluoroethyl, 1-methyl-2,2,2-trifluoroethyl, alkyl, substituted alkyl, aryl, substituted aryl, heterocycle, or substituted heterocycle;
R2 is 1,1-dimethylethyl, 1-methyl-1-fluoroethyl, 1-methylethyl, 1,1-difluoroethyl, 1-fluoroethyl, trifluoromethyl, difluoromethyl, 1-methyl-2-fluoroethyl, hydrogen, halogen, alkyl, substituted alkyl, aryl, substituted aryl, heterocycle, or substituted heterocycle;
R3 is gamma-lactamyl, cyclopentanonyl, cyclopentyl, beta-lactamyl, cyclobutanonyl, cyclobutyl, acetonyl, acetyl, gamma-lactonyl, furanonyl, pyrrolonyl, cyclopentenonyl, oxazolonyl, imidazolonyl, alkyl, substituted alkyl, aryl, substituted aryl, heterocycle, or substituted heterocycle;
R4 is hydrogen, halomethyl, hydroxymethyl, diazomethyl, acyloxymethyl, ketone, ketoamide, ketoacid, ketoester, alkyl, substituted alkyl aryl, substituted aryl, heterocycle, or substituted heterocycle;
X is CH₂, NH, or O; and
the bond indicated by the asterisk is a single or double bond.

In certain embodiments, a compound of the present disclosure has the formula:

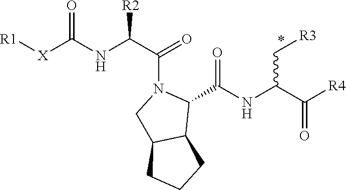

wherein:
R1 is 1,1-dimethylethyl, 1-methyl-1-fluoroethyl, 1-methylethyl, 1,1-difluoroethyl, 1-fluoroethyl, ethyl, trifluoromethyl, difluoromethyl, fluoromethyl, methyl, 2-fluoroethyl, 2,2-difluoroethyl, 2,2,2-trifluoroethyl, 1-methyl-2-fluoroethyl, 1-methyl-2,2-difluoroethyl, 1-methyl-2,2,2-trifluoroethyl, alkyl, substituted alkyl, aryl, substituted aryl, heterocycle, or substituted heterocycle;
R2 is 1,1-dimethylethyl, 1-methyl-1-fluoroethyl, 1-methylethyl, 1,1-difluoroethyl, 1-fluoroethyl, trifluoromethyl, difluoromethyl, 1-methyl-2-fluoroethyl, hydrogen, halogen, alkyl, substituted alkyl, aryl, substituted aryl, heterocycle, or substituted heterocycle;
R3 is gamma-lactamyl, cyclopentanonyl, cyclopentyl, beta-lactamyl, cyclobutanonyl, cyclobutyl, acetonyl, acetyl, gamma-lactonyl, furanonyl, pyrrolonyl, cyclopentenonyl, oxazolonyl, imidazolonyl, alkyl, substituted alkyl, aryl, substituted aryl, heterocycle, or substituted heterocycle;
R4 is hydrogen, halomethyl, hydroxymethyl, diazomethyl, acyloxymethyl, ketone, ketoamide, ketoacid, ketoester, alkyl, substituted alkyl aryl, substituted aryl, heterocycle, or substituted heterocycle;
X is CH₂, NH, or O; and
the bond indicated by the asterisk is a single or double bond.

According to some embodiments, a compound of the present disclosure has the formula:

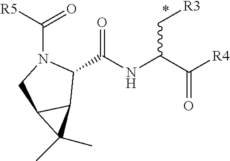

wherein:
R5 is pyrazole, substituted pyrazole, imidazole, substituted imidazole, piperidine, substituted piperidine, pyridine, substituted pyridine, pyridone, substituted pyridone, indole, methoxyindole, other substituted indole, isoindole, substituted isoindole, purine, substituted purine, benzyloxy, substituted benzyloxy, benzylamino, substituted benzylamino, phenoxymethyl, substituted phenoxymethyl, phenylaminomethyl, substituted phenylaminomethyl, phenylethyl, substituted phenylethyl, alkyl, substituted alkyl, aryl, substituted aryl, heterocycle, or substituted heterocycle;
R3 is gamma-lactamyl, cyclopentanone, cyclopentyl, beta-lactamyl, cyclobutanonyl, cyclobutyl, acetonyl, acetyl, gamma-lactonyl, furanonyl, pyrrolonyl, oxazolonyl, imidazolonyl, cyclopentenonyl, alkyl, substituted alkyl, aryl, substituted aryl, heterocycle, or substituted heterocycle;
R4 is hydrogen, halomethyl, hydroxymethyl, diazomethyl, acyloxymethyl, ketone, ketoamide, ketoacid, ketoester, alkyl, substituted alkyl aryl, substituted aryl, heterocycle, or substituted heterocycle; and
the bond indicated by the asterisk is a single or double bond.

In certain embodiments, a compound of the present disclosure has the formula:

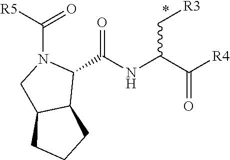

wherein:
R5 is pyrazole, substituted pyrazole, imidazole, substituted imidazole, piperidine, substituted piperidine, pyridine, substituted pyridine, pyridone, substituted pyridone, indole, methoxyindole, other substituted indole, isoindole, substituted isoindole, purine, substituted purine, benzyloxy, substituted benzyloxy, benzylamino, substituted benzylamino, phenoxymethyl, substituted phenoxymethyl, phenylaminomethyl, substituted phenylaminomethyl, phenylethyl, substituted phenylethyl, alkyl, substituted alkyl, aryl, substituted aryl, heterocycle, or substituted heterocycle;
R3 is gamma-lactamyl, cyclopentanone, cyclopentyl, beta-lactamyl, cyclobutanonyl, cyclobutyl, acetonyl, acetyl, gamma-lactonyl, furanonyl, pyrrolonyl, oxazolonyl, imidazolonyl, cyclopentenonyl, alkyl, substituted alkyl, aryl, substituted aryl, heterocycle, or substituted heterocycle;
R4 is hydrogen, halomethyl, hydroxymethyl, diazomethyl, acyloxymethyl, ketone, ketoamide, ketoacid, ketoester, alkyl, substituted alkyl aryl, substituted aryl, heterocycle, or substituted heterocycle; and
the bond indicated by the asterisk is a single or double bond.

According to some embodiments, a compound of the present disclosure has the formula:

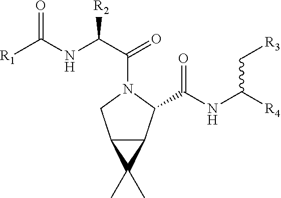

wherein:
R₁ and R₂ are independently alkyl, cycloalkyl, substituted alkyl, heteroalkyl, or heterocycloalkyl;
R₃ is gamma-lactamyl, cyclopentanone, cyclopentyl, beta-lactamyl, cyclobutanonyl, cyclobutyl, acetonyl, acetyl, gamma-lactonyl, furanonyl, pyrrolonyl, oxazolonyl, imidazolonyl, cyclopentenonyl, alkyl, substituted alkyl, aryl, substituted aryl, heterocycle, or substituted heterocycle; and
R₄ is primary alpha-ketoamide, secondary alpha-ketoamide, or tertiary alpha-ketoamide.

In certain embodiments, a compound of the present disclosure has the formula:

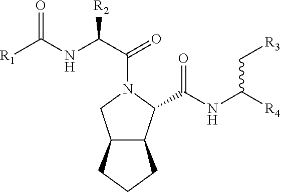

wherein:
R₁ and R₂ are independently alkyl, cycloalkyl, substituted alkyl, heteroalkyl, or heterocycloalkyl;
R₃ is gamma-lactamyl, cyclopentanone, cyclopentyl, beta-lactamyl, cyclobutanonyl, cyclobutyl, acetonyl, acetyl, gamma-lactonyl, furanonyl, pyrrolonyl, oxazolonyl, imidazolonyl, cyclopentenonyl, alkyl, substituted alkyl, aryl, substituted aryl, heterocycle, or substituted heterocycle; and
R₄ is primary alpha-ketoamide, secondary alpha-ketoamide, or tertiary alpha-ketoamide.

According to some embodiments, a compound of the present disclosure has the formula:

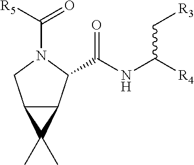

wherein:
R₃ is gamma-lactamyl, cyclopentanone, cyclopentyl, beta-lactamyl, cyclobutanonyl, cyclobutyl, acetonyl, acetyl, gamma-lactonyl, furanonyl, pyrrolonyl, oxazolonyl, imidazolonyl, cyclopentenonyl, alkyl, substituted alkyl, aryl, substituted aryl, heterocycle, or substituted heterocycle;
R₄ is primary alpha-ketoamide, secondary alpha-ketoamide, tertiary alpha-ketoamide, or nitrile; and
R₅ is pyrazole, substituted pyrazole, imidazole, substituted imidazole, piperidine, substituted piperidine, pyridine, substituted pyridine, pyridone, substituted pyridone, indole, methoxyindole, other substituted indole, isoindole, substituted isoindole, purine, substituted purine, benzyloxy, substituted benzyloxy, benzylamino, substituted benzylamino, phenoxymethyl, substituted phenoxymethyl, phenylaminomethyl, substituted phenylaminomethyl, phenylethyl, substituted phenylethyl, phenylmethyl, substituted phenylmethyl, alkyl, substituted alkyl, aryl, substituted aryl, heterocycle, or substituted heterocycle.

In certain embodiments, a compound of the present disclosure has the formula:

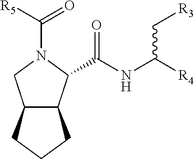

wherein:
R₃ is gamma-lactamyl, cyclopentanone, cyclopentyl, beta-lactamyl, cyclobutanonyl, cyclobutyl, acetonyl, acetyl, gamma-lactonyl, furanonyl, pyrrolonyl, oxazolonyl, imidazolonyl, cyclopentenonyl, alkyl, substituted alkyl, aryl, substituted aryl, heterocycle, or substituted heterocycle;
R₄ is primary alpha-ketoamide, secondary alpha-ketoamide, tertiary alpha-ketoamide, or nitrile; and
R₅ is pyrazole, substituted pyrazole, imidazole, substituted imidazole, piperidine, substituted piperidine, pyridine, substituted pyridine, pyridone, substituted pyridone, indole, methoxyindole, other substituted indole, isoindole, substituted isoindole, purine, substituted purine, benzyloxy, substituted benzyloxy, benzylamino, substituted benzylamino, phenoxymethyl, substituted phenoxymethyl, phenylaminomethyl, substituted phenylaminomethyl, phenylethyl, substituted phenylethyl, phenylmethyl, substituted phenylmethyl, alkyl, substituted alkyl, aryl, substituted aryl, heterocycle, or substituted heterocycle.

According to some embodiments, a compound of the present disclosure has the formula:

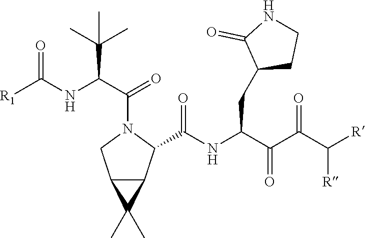

wherein:
R₁ is alkyl, cycloalkyl, substituted alkyl, heteroalkyl, or heterocycloalkyl; and
R′ and R″ are independently H, alkyl, aryl, heteroalkyl, alkenyl, alkynyl, heteroaryl, cycloalkyl, heterocyclyl, arylalkyl, heteroarylalkyl, or substituted alkyl,
optionally wherein R′ and R″ are interconnected.

In certain embodiments, for such a compound, R₁ is bicyclo[1.1.1]pentanyl.

According to some embodiments, the compound is:

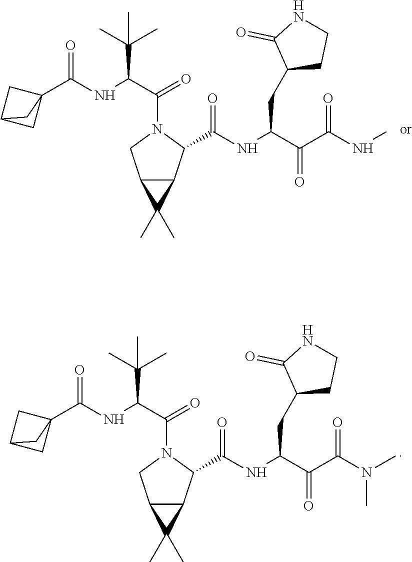 [SMILES: CNC(=O)C(=O)[C@H](C[C@@H]1CCNC1=O)NC(=O)[C@@H]1[C@@H]2[C@H](CN1C(=O)[C@@H](NC(=O)C13CC(C1)C3)C(C)(C)C)C2(C)C ; CN(C)C(=O)C(=O)[C@H](C[C@@H]1CCNC1=O)NC(=O)[C@@H]1[C@@H]2[C@H](CN1C(=O)[C@@H](NC(=O)C13CC(C1)C3)C(C)(C)C)C2(C)C]

In certain embodiments, for such a compound, R₁ is neopentanyl.

According to some embodiments, the compound is:

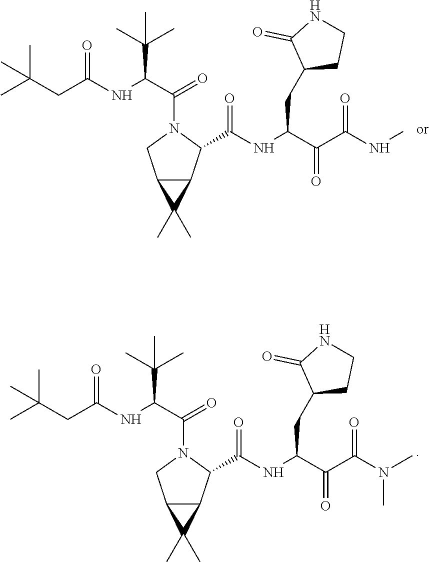

In certain embodiments, for such a compound, R′ and R″ are each methyl. For example, according to some embodiments, the compound is:

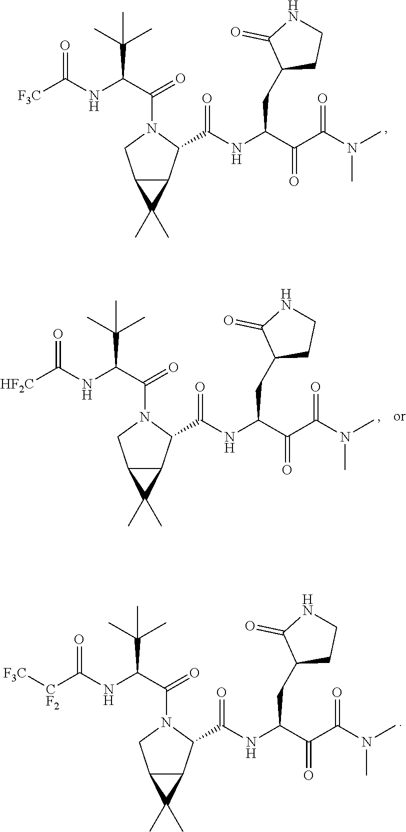

In certain embodiments, the compound is:

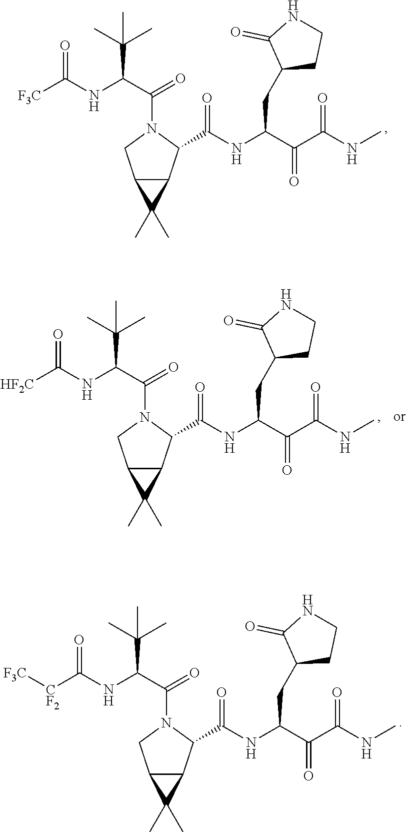

According to some embodiments, a compound of the present disclosure has the formula:

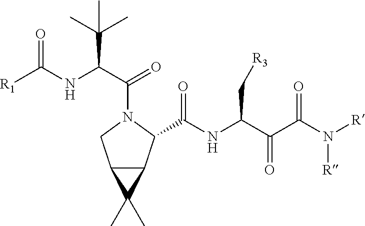

wherein:
R₁ is alkyl, cycloalkyl, substituted alkyl, heteroalkyl, or heterocycloalkyl;
R₃ is pyridinyl, substituted pyridinyl, phenyl, substituted phenyl, heteroaryl, or heterocyclyl; and
R′ and R″ are independently H, alkyl, aryl, heteroalkyl, alkenyl, alkynyl, heteroaryl, cycloalkyl, heterocyclyl, arylalkyl, heteroarylalkyl, or substituted alkyl,
optionally wherein R′ and R″ are interconnected.

In certain embodiments, for such a compound, R₁ is neopentanyl, R₃ is pyridinyl, and R′ and R″ are independently H or methyl. According to some embodiments, the compound is:

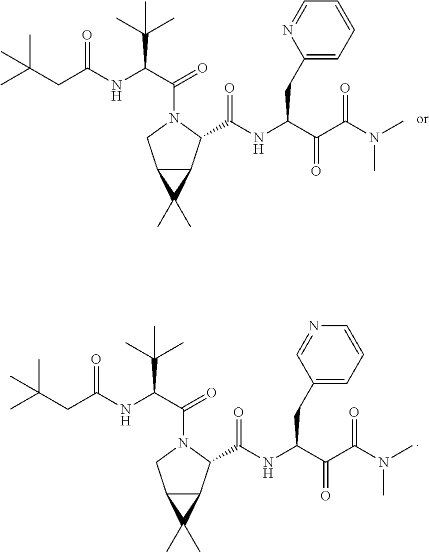

In certain embodiments, the compound is:

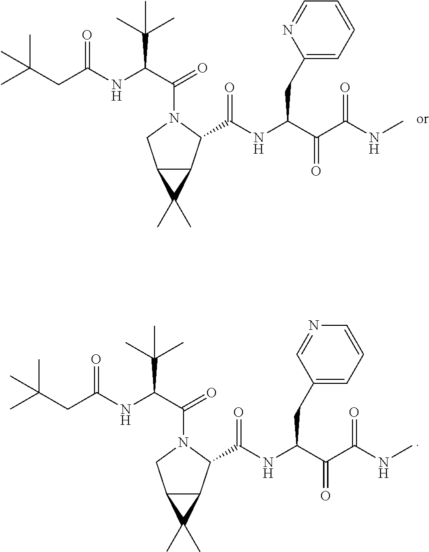

According to some embodiments, R₁ is bicyclo[1.1.1]pentanyl, Ra is pyridinyl, and R′ and R″ are independently H or methyl. In certain embodiments, the compound is:

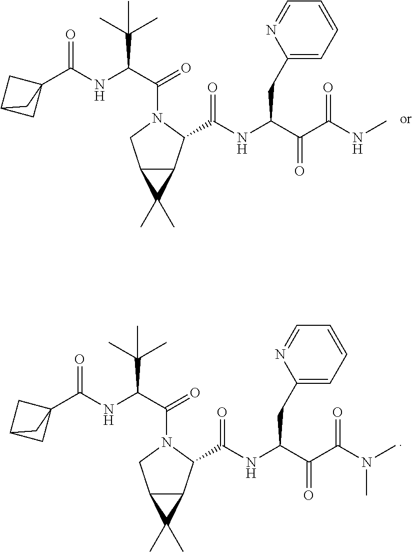 [SMILES: CNC(=O)C(=O)[C@H](Cc1ccccn1)NC(=O)[C@@H]1[C@@H]2[C@H](CN1C(=O)[C@@H](NC(=O)C13CC(C1)C3)C(C)(C)C)C2(C)C ; CN(C)C(=O)C(=O)[C@H](Cc1ccccn1)NC(=O)[C@@H]1[C@@H]2[C@H](CN1C(=O)[C@@H](NC(=O)C13CC(C1)C3)C(C)(C)C)C2(C)C]

According to some embodiments, the compound is:

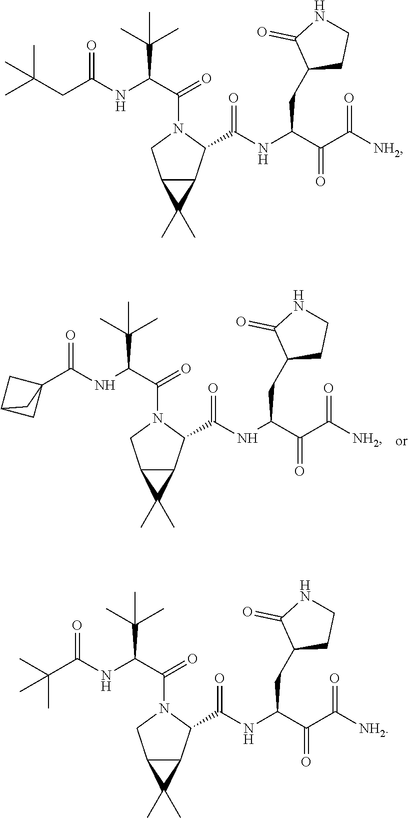 [SMILES: CC(C)(C)CC(=O)N[C@H](C(=O)N1C[C@H]2[C@@H]([C@H]1C(=O)N[C@@H](C[C@@H]1CCNC1=O)C(=O)C(N)=O)C2(C)C)C(C)(C)C ; CC(C)(C)[C@H](NC(=O)C12CC(C1)C2)C(=O)N1C[C@H]2[C@@H]([C@H]1C(=O)N[C@@H](C[C@@H]1CCNC1=O)C(=O)C(N)=O)C2(C)C ; CC(C)(C)C(=O)N[C@H](C(=O)N1C[C@H]2[C@@H]([C@H]1C(=O)N[C@@H](C[C@@H]1CCNC1=O)C(=O)C(N)=O)C2(C)C)C(C)(C)C]

According to some embodiments, a compound of the present disclosure has the formula:

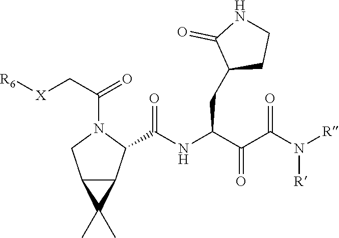

wherein:
X is CH₂, NH, or O;
R₆ is aryl, heteroaryl, substituted aryl, or substituted heteroaryl; and
R′ and R″ are independently H, alkyl, aryl, heteroalkyl, alkenyl, alkynyl, heteroaryl, cycloalkyl, heterocyclyl, arylalkyl, heteroarylalkyl, or substituted alkyl,
optionally wherein R′ and R″ are interconnected.

In certain embodiments, for such a compound, X is CH₂ and R₆ is substituted phenyl.

According to some embodiments, the compound is:

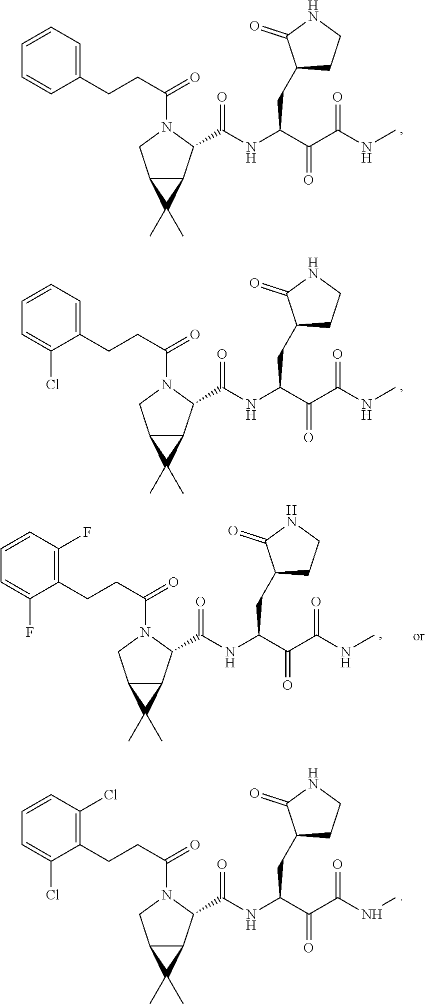 [SMILES: CNC(=O)C(=O)[C@H](C[C@@H]1CCNC1=O)NC(=O)[C@@H]1[C@@H]2[C@H](CN1C(=O)CCc1ccccc1)C2(C)C ; CNC(=O)C(=O)[C@H](C[C@@H]1CCNC1=O)NC(=O)[C@@H]1[C@@H]2[C@H](CN1C(=O)CCc1ccccc1Cl)C2(C)C ; CNC(=O)C(=O)[C@H](C[C@@H]1CCNC1=O)NC(=O)[C@@H]1[C@@H]2[C@H](CN1C(=O)CCc1c(F)cccc1F)C2(C)C ; CNC(=O)C(=O)[C@H](C[C@@H]1CCNC1=O)NC(=O)[C@@H]1[C@@H]2[C@H](CN1C(=O)CCc1c(Cl)cccc1Cl)C2(C)C]

In certain embodiments, X is NH and R₆ is substituted phenyl. For example, the compound may be:

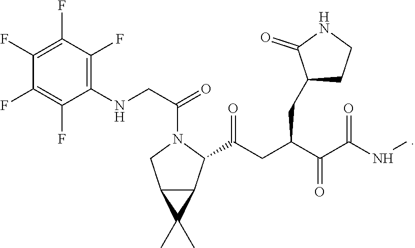 [SMILES: CNC(=O)C(=O)[C@@H](CC(=O)[C@@H]1[C@@H]2[C@H](CN1C(=O)CNc1c(F)c(F)c(F)c(F)c1F)C2(C)C)C[C@@H]1CCNC1=O]

According to some embodiments, R₆ is trihalophenyl. For example, the compound may be:

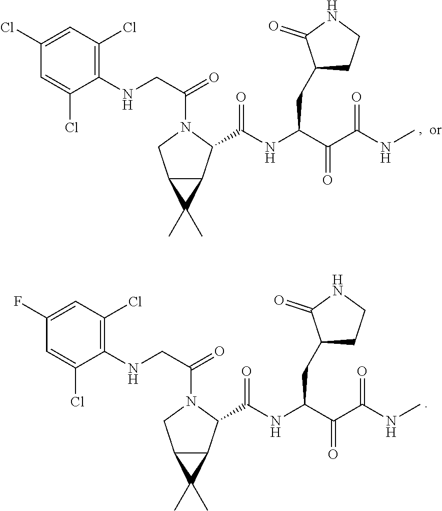 [SMILES: CNC(=O)C(=O)[C@H](C[C@@H]1CCNC1=O)NC(=O)[C@@H]1[C@@H]2[C@H](CN1C(=O)CNc1c(Cl)cc(Cl)cc1Cl)C2(C)C ; CNC(=O)C(=O)[C@H](C[C@@H]1CCNC1=O)NC(=O)[C@@H]1[C@@H]2[C@H](CN1C(=O)CNc1c(Cl)cc(F)cc1Cl)C2(C)C]

In certain embodiments, R₆ is trifluorophenyl. For example, the compound may be:

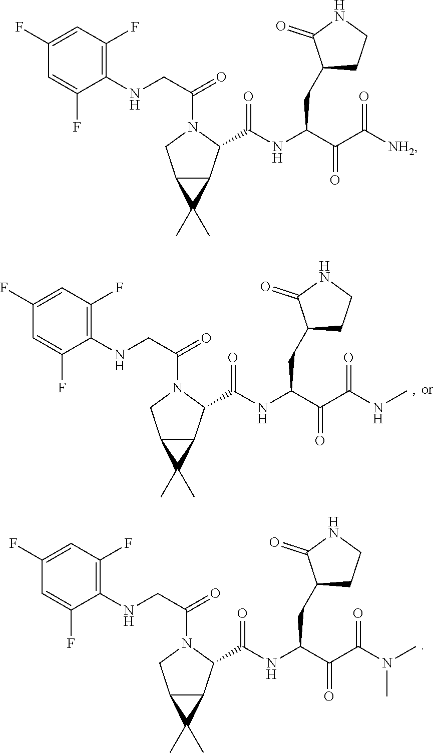 [SMILES: CC1(C)[C@@H]2[C@@H](C(=O)N[C@@H](C[C@@H]3CCNC3=O)C(=O)C(N)=O)N(C(=O)CNc3c(F)cc(F)cc3F)C[C@@H]21 ; CNC(=O)C(=O)[C@H](C[C@@H]1CCNC1=O)NC(=O)[C@@H]1[C@@H]2[C@H](CN1C(=O)CNc1c(F)cc(F)cc1F)C2(C)C ; CN(C)C(=O)C(=O)[C@H](C[C@@H]1CCNC1=O)NC(=O)[C@@H]1[C@@H]2[C@H](CN1C(=O)CNc1c(F)cc(F)cc1F)C2(C)C]

According to some embodiments, X is O and R₆ is substituted phenyl. In certain embodiments, R₆ is trihalophenyl. For example, the compound may be:

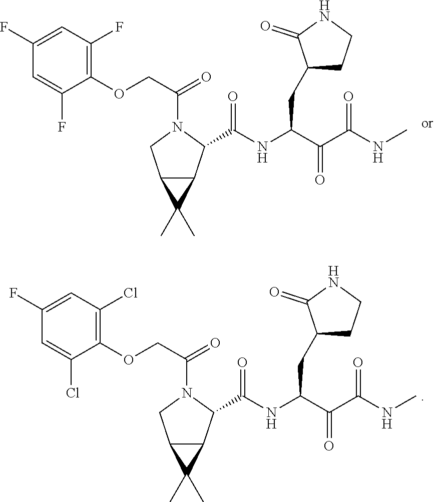 [SMILES: CNC(=O)C(=O)[C@H](C[C@@H]1CCNC1=O)NC(=O)[C@@H]1[C@@H]2[C@H](CN1C(=O)COc1c(F)cc(F)cc1F)C2(C)C ; CNC(=O)C(=O)[C@H](C[C@@H]1CCNC1=O)NC(=O)[C@@H]1[C@@H]2[C@H](CN1C(=O)COc1c(Cl)cc(F)cc1Cl)C2(C)C]

In certain embodiments, a compound of the present disclosure has the formula:

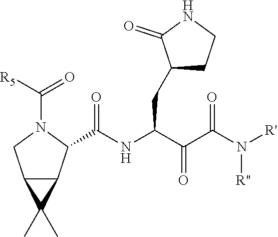

wherein:
R₅ is alkyl, substituted alkyl, aryl, heteroalkyl, heteroaryl, cycloalkyl, heterocyclyl, arylalkyl, cyclylalkyl, heterocyclylalkyl, arylheteroalkyl, heteroarylalkyl, substituted arylalkyl, substituted heteroaryl, or substituted heteroarylalkyl; and
R′ and R″ are independently H, alkyl, aryl, heteroalkyl, alkenyl, alkynyl, heteroaryl, cycloalkyl, heterocyclyl, arylalkyl, heteroarylalkyl, or substituted alkyl,
optionally wherein R′ and R″ are interconnected.

According to some embodiments, such a compound is:

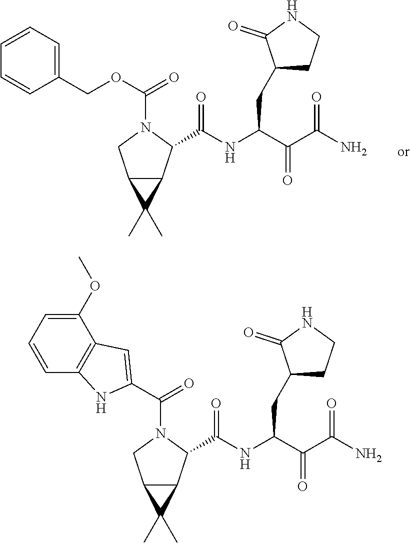 [SMILES: CC1(C)[C@@H]2[C@@H](C(=O)N[C@@H](C[C@@H]3CCNC3=O)C(=O)C(N)=O)N(C(=O)OCc3ccccc3)C[C@@H]21 ; COc1cccc2[nH]c(C(=O)N3C[C@H]4[C@@H]([C@H]3C(=O)N[C@@H](C[C@@H]3CCNC3=O)C(=O)C(N)=O)C4(C)C)cc12]

In certain embodiments, for such a compound, R′ is methyl and R″ is H or methyl.

According to some embodiments, a compound of the present disclosure has the formula:

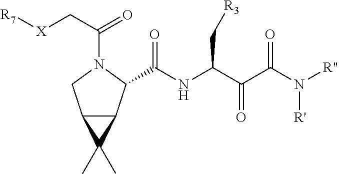

wherein:
X is CH₂, NH, or O;
R₃ is pyridinyl, substituted pyridinyl, phenyl, substituted phenyl, heteroaryl, or heterocyclyl;
R₇ is a substituted phenyl; and
R′ and R″ are independently H, alkyl, aryl, heteroalkyl, alkenyl, alkynyl, heteroaryl, cycloalkyl, heterocyclyl, arylalkyl, heteroarylalkyl, or substituted alkyl,
optionally wherein R′ and R″ are interconnected.

In certain embodiments, for such a compound, X is NH and R₇ is a trihalophenyl.

According to some embodiments, R₇ is a trifluorophenyl. In certain embodiments, R₃ is pyridinyl. According to some embodiments, the compound is:

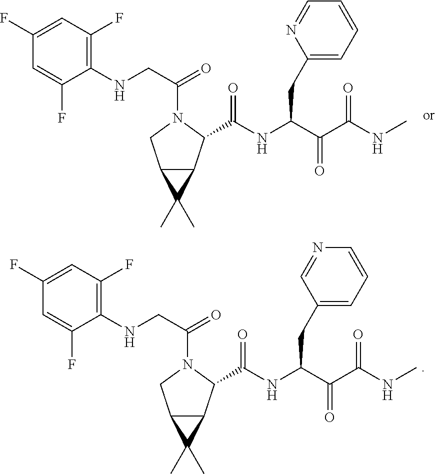 [SMILES: CNC(=O)C(=O)[C@H](Cc1ccccn1)NC(=O)[C@@H]1[C@@H]2[C@H](CN1C(=O)CNc1c(F)cc(F)cc1F)C2(C)C ; CNC(=O)C(=O)[C@H](Cc1cccnc1)NC(=O)[C@@H]1[C@@H]2[C@H](CN1C(=O)CNc1c(F)cc(F)cc1F)C2(C)C]

In certain embodiments, a compound of the present disclosure has the formula:

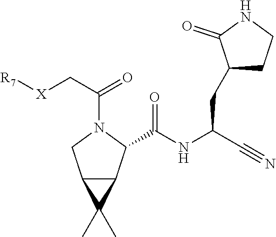

wherein:
X is CH₂, NH, or O; and
R₇, is a substituted phenyl.

For example, the compound may be:

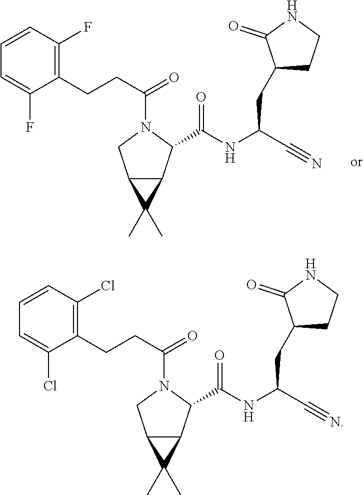 [SMILES: CC1(C)[C@@H]2[C@@H](C(=O)N[C@H](C#N)C[C@@H]3CCNC3=O)N(C(=O)CCc3c(F)cccc3F)C[C@@H]21 ; CC1(C)[C@@H]2[C@@H](C(=O)N[C@H](C#N)C[C@@H]3CCNC3=O)N(C(=O)CCc3c(Cl)cccc3Cl)C[C@@H]21]

According to some embodiments, X is NH and R₇ is a trihalophenyl. For example, the compound may be:

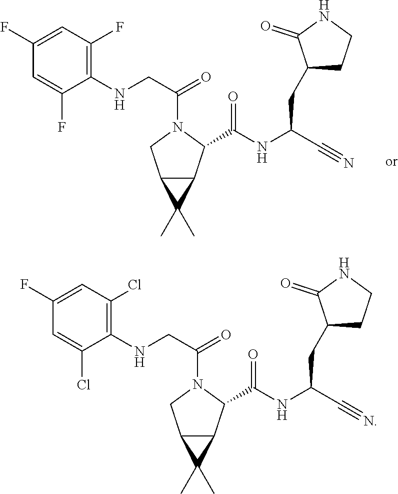 [SMILES: CC1(C)[C@@H]2[C@@H](C(=O)N[C@H](C#N)C[C@@H]3CCNC3=O)N(C(=O)CNc3c(F)cc(F)cc3F)C[C@@H]21 ; CC1(C)[C@@H]2[C@@H](C(=O)N[C@H](C#N)C[C@@H]3CCNC3=O)N(C(=O)CNc3c(Cl)cc(F)cc3Cl)C[C@@H]21]

In certain embodiments, X is O and R₇ is a trihalophenyl. For example, the compound may be:

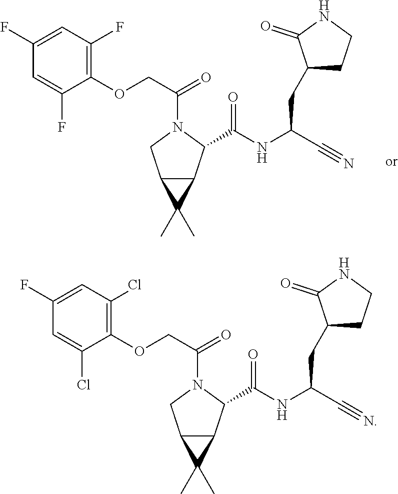 [SMILES: CC1(C)[C@@H]2[C@@H](C(=O)N[C@H](C#N)C[C@@H]3CCNC3=O)N(C(=O)COc3c(F)cc(F)cc3F)C[C@@H]21 ; CC1(C)[C@@H]2[C@@H](C(=O)N[C@H](C#N)C[C@@H]3CCNC3=O)N(C(=O)COc3c(Cl)cc(F)cc3Cl)C[C@@H]21]

The compounds of the present disclosure may be synthesized using any suitable synthetic scheme. Non-limiting examples of approaches for synthesizing example compounds of the present disclosure will now be described.

### Synthesis Example for ML1001—General Example of Tetrapeptides with a Primary Ketoamide

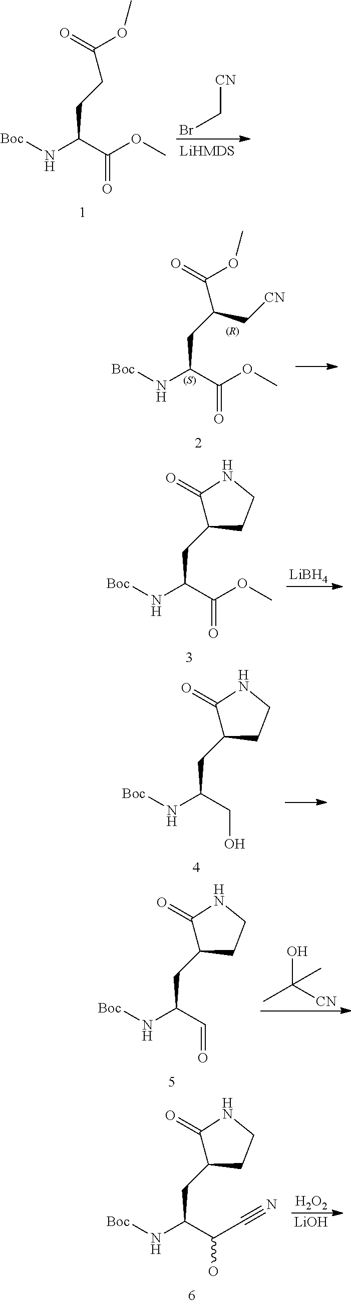

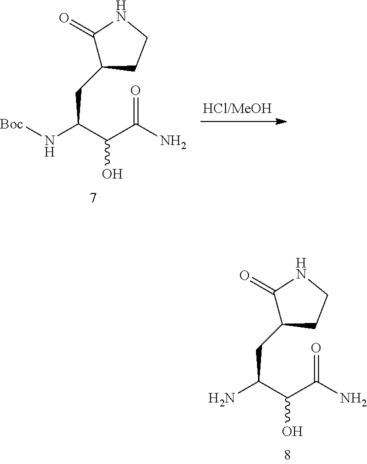 [SMILES: CC(C)(C)OC(=O)N[C@@H](C[C@@H]1CCNC1=O)C(O)C(N)=O ; NC(=O)C(O)[C@@H](N)C[C@@H]1CCNC1=O]

Stereoselective dianionic alkylation of 1 with bromoacetonitrile using LiHMDS provided 2 (J. Med. Chem., 2020, 63, 4562-4578). Next, the nitrile of 2 was subjected to hydrogenation using Raney Ni as a catalyst, which was followed by in situ cyclization to yield. 3. The ester group of 3 was then reduced using LiBH₄ to yield the primary alcohol of 4. The alcohol of 4 was oxidized in a Parikh-Doering oxidation yielding the aldehyde 5. Cyanation of the aldehyde with 2-hydroxy-2-methylpropanenitrile provided 6. The nitrile of 6 was converted to the corresponding amide of 7 using H₂O₂ and LiOH. In preparation for condensation, the Boc protection group was removed from 7 in hydrochloride methanol.

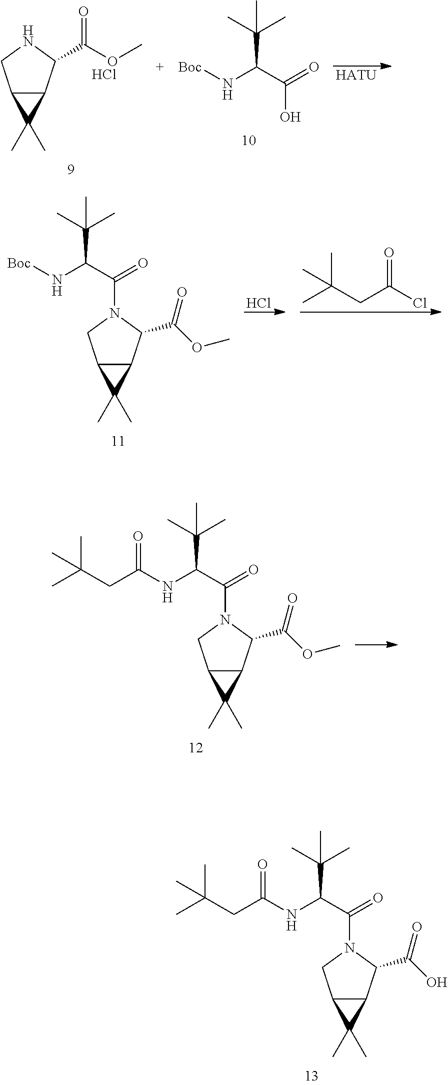

Condensation of 9 and 10 in the presence of HATU to yield 11. Following deprotection (intermediate not shown), condensation with 3,3-dimethylbutanoyl chloride provided 12. Hydrolysis of the ester yielded 13.

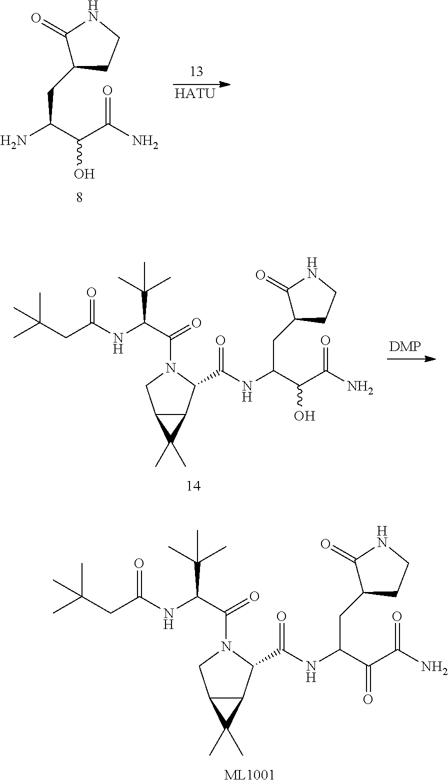

Condensation of 8 and 13 was achieved using HATU and yielded 14. From 14, the final product ML1001 was obtained by oxidation with Dess-Martin Periodinane.

Those skilled in the art will realize that the synthesis of analogous of 13 allows access to the general formula I.

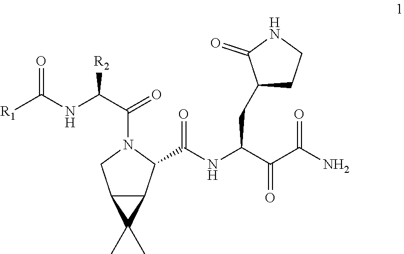

In particular, this method has also been used for the synthesis of ML1002 and ML1003.

### Synthesis Example for ML104—General Example for Tripeptides with a Primary Ketoamide

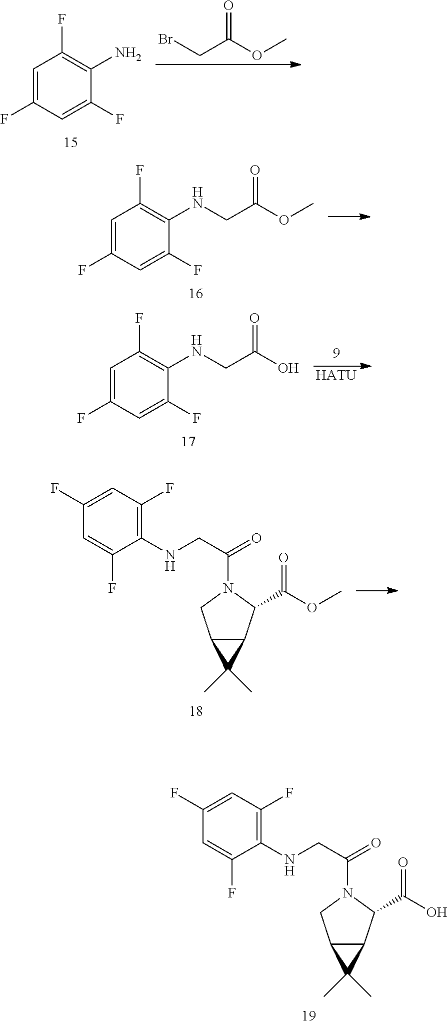

2,4,6-trifluoroaniline, 15, underwent nucleophilic substitution with methyl 2-bromoacetate to yield 16. The ester of 16 was hydrolyzed to its carboxylic acid 17. A condensation reaction of 17 and 9 in the presence of HATU yielded 18. Hydrolysis of the ester yielded 19.

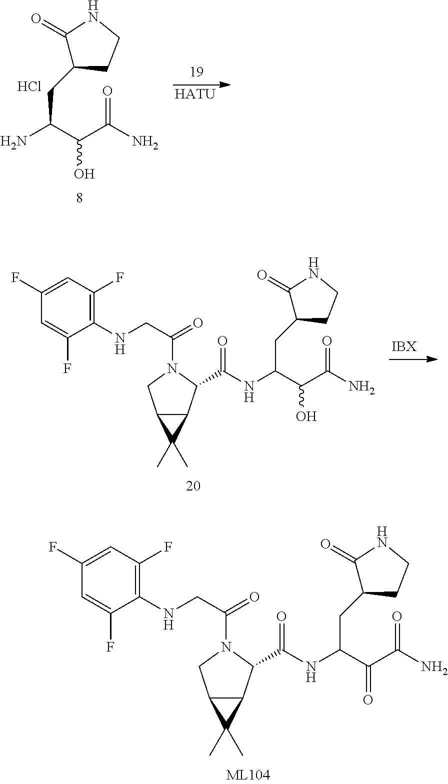

Condensation of 8 and 19 was achieved using HATU and yielded 20. From 20, the final product ML104 was obtained by oxidation with 2-iodoxybenzoic acid.

Those of ordinary skill in the art will recognize that other compounds of the general formula II are accessible by exchanging the intermediate 17 in the synthesis above.

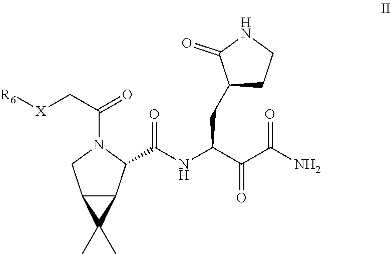

X is CH₂, NH, O; and R₆ is substituted phenyl.

For example, ML102 was obtained using the same method by substituting 17 for 3-phenylpropanoic acid. Synthesis of other substituted phenylglycine intermediates (i.e. X is NH) are achieved by reaction of substituted anilines with methyl 2-bromoacetate. Similarly, substituted 2-phenoxyacetic acid (i.e. X is O) are generated by reaction of substituted phenolates with methyl 2-bromoacetate.

More generally, compounds of formula III are available through this approach when R₅ is available as a carboxylic acid derivative.

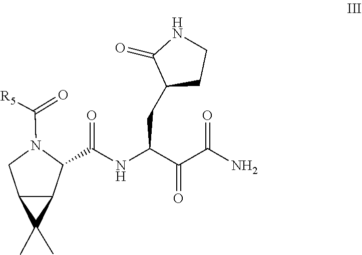

### Synthesis Examples for ML104m, General Example for Compounds with Secondary Ketoamides

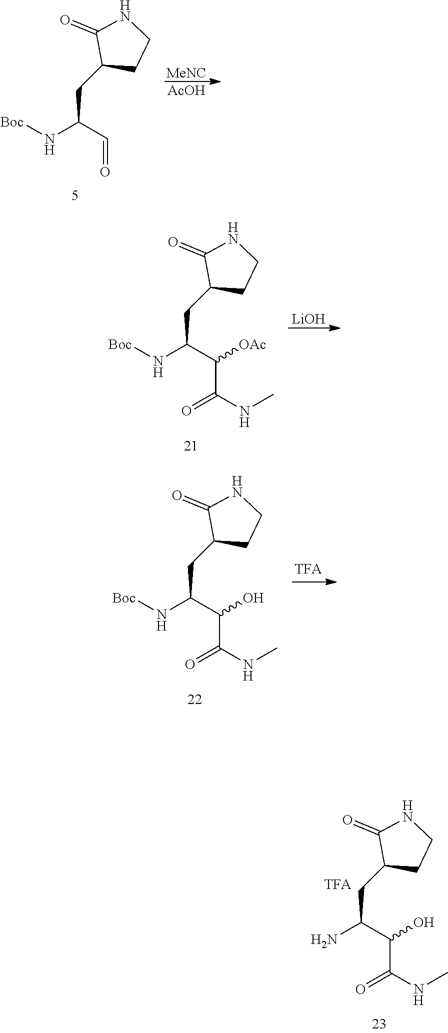

Nucleophilic addition of isocyanomethane to 5 in the presence of acetic acid yielded 21 that upon treatment with LiOH provided 22. Deprotection of 22 with TFA yielded 23.

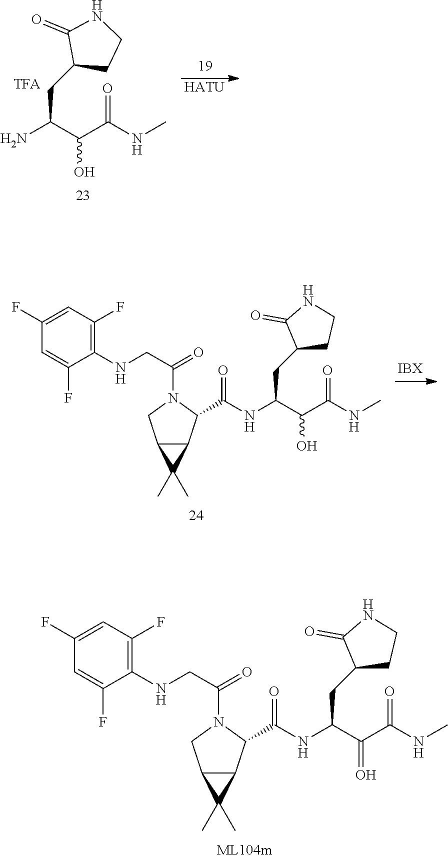

Condensation of 19 and 23 was achieved using HATU and yielded 24. From 24, the final product ML104m was obtained by oxidation with 2-iodoxybenzoic acid.

Comparable protocols have been used to obtain ML102m and ML1002m by exchanging intermediate 19 in the condensation reaction with 23.

Those of ordinary skill in the art will recognize that, in place of 23, other secondary ketoamide intermediates of the formula IV are available by substituting isocyanomethane with other isocyano-R′ compounds in the synthesis of 21. Here R′ is alkyl-, aryl-, heteroalkyl-, alkenyl-, alkynyl-, heteroaryl-, cycloalkyl-, heterocyclyl-, arylalkyl-, and heteroarylalkyl. This is for example seen in J. Med. Chem., 2020, 63, 4562-4578 and Science, 2020, 368, 409-412.

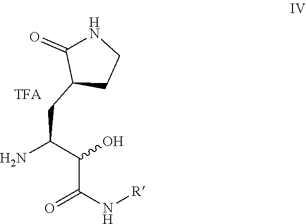

### Generalizable Synthesis Schemes for Compounds Containing Tertiary (and Secondary) Ketoamides

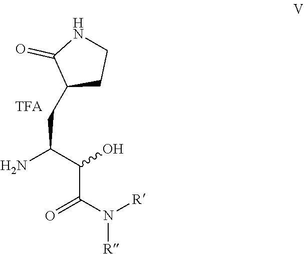

Tertiary ketoamides of interest are available by exchanging 8 and 23 in the syntheses described above. Described here are two methods for generation of tertiary ketoamides intermediates of the general formula V, wherein R′ and R″ are alkyl-, aryl-, heteroalkyl-, alkenyl-, alkynyl-, heteroaryl-, cycloalkyl-, heterocyclyl-, arylalkyl-, heteroarylalkyl, or substituted alkyl,
optionally wherein R′ and R″ are interconnected.

Method A

This synthesis is based on the approach reported in J. Med. Chem. Let., 2019, 10, 1086-92. As an example, described here is the synthesis of ML104d, a N,N-dimethylated tertiary ketoamide.

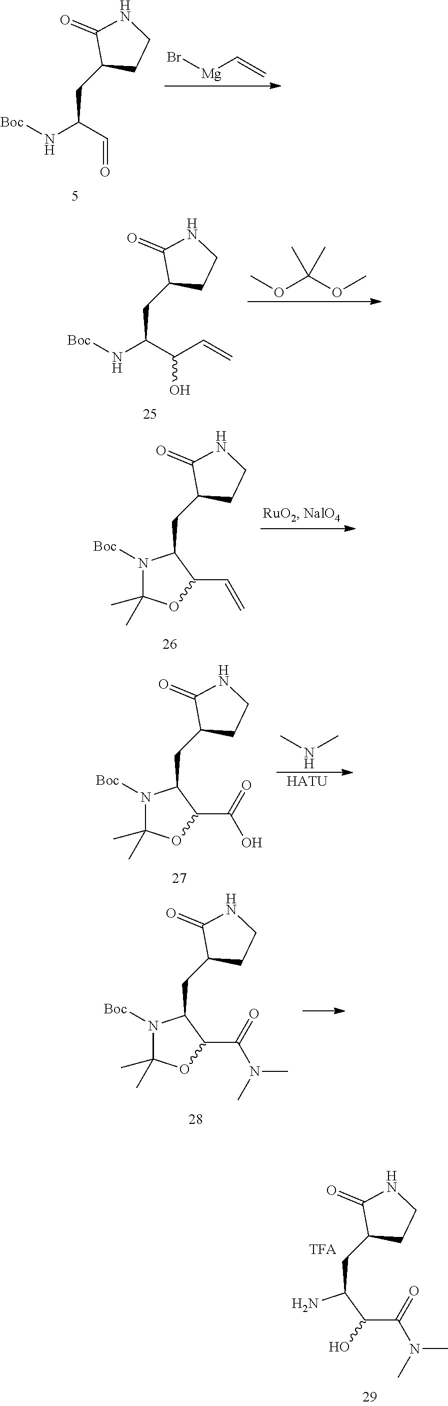

The vinyl derivative of 5, 25, is obtained by reaction with vinylmagnesium bromide. Protection with 2,2-dimethoxypropane leads to 26. Oxidative cleavage of the alkene of 26 with RuO₂ and NaIO₄ affords the carboxylic acid 27. Using HATU, 27 reacts with dimethylamine to yield 28. Deprotection of 28 with TFA provides 29.

The protection step using 2,2-dimethoxypropane is expected to be beneficial, although not necessary to successfully reach 29.

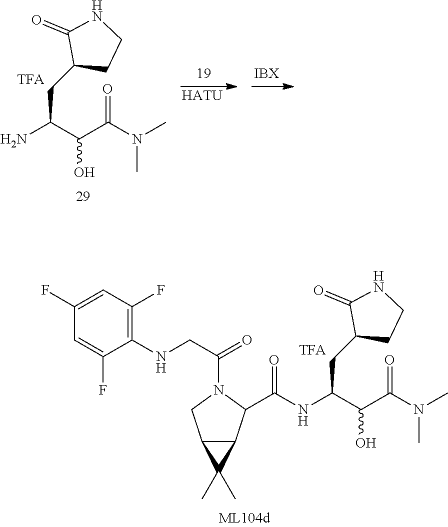

Condensation of 19 and 29 using HATU, followed by oxidation of the intermediate with 2-iodoxybenzoic acid yields ML104d.

Other variants of V are accessible by substituting the dimethylamine in the example above with appropriate secondary or primary amines. Use of primary amines in this method allows access to secondary ketoamides, variants of IV, that are not available from the isocyanide route described above.

Method B

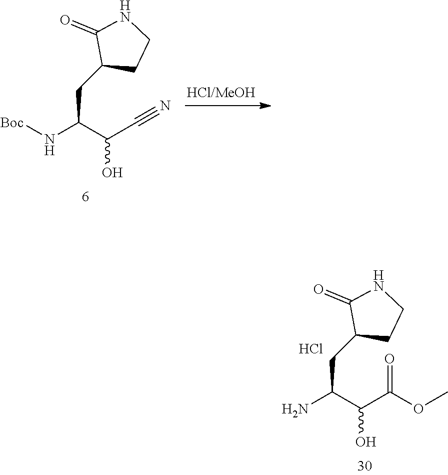 [SMILES: CC(C)(C)OC(=O)N[C@@H](C[C@@H]1CCNC1=O)C(O)C#N ; COC(=O)C(O)[C@@H](N)C[C@@H]1CCNC1=O ; Cl]

In another method to obtain tertiary ketoamides, the nitrile of 6 is hydrolyzed to the corresponding methyl ester and the Boc protection group is simultaneously removed to yield 30.

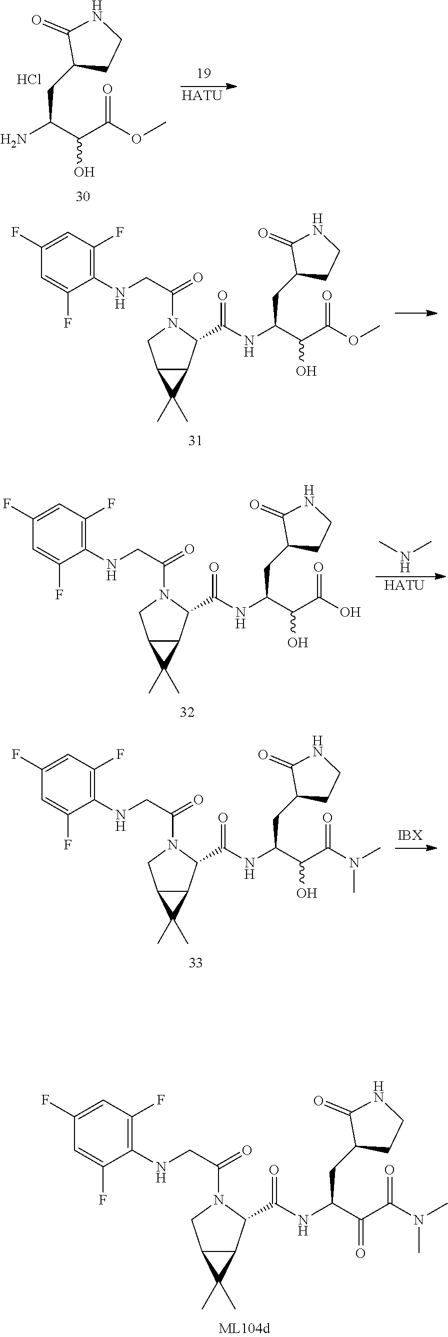

A condensation of 30 and 19 provides 31, that is hydrolyzed to the carboxylic acid of 32. In the presence of HATU, reaction of 32 with dimethylamine yields 33. A final oxidation using 2-iodoxybenzoic acid provides the target molecule ML104d.

### Synthesis Example for ML104N, as a Generalizable Example for Nitrile Containing Compounds

Nitrile warheads are available starting from the intermediate 3 based on syntheses in J. Org. Chem., 2003, 68, 50-54.

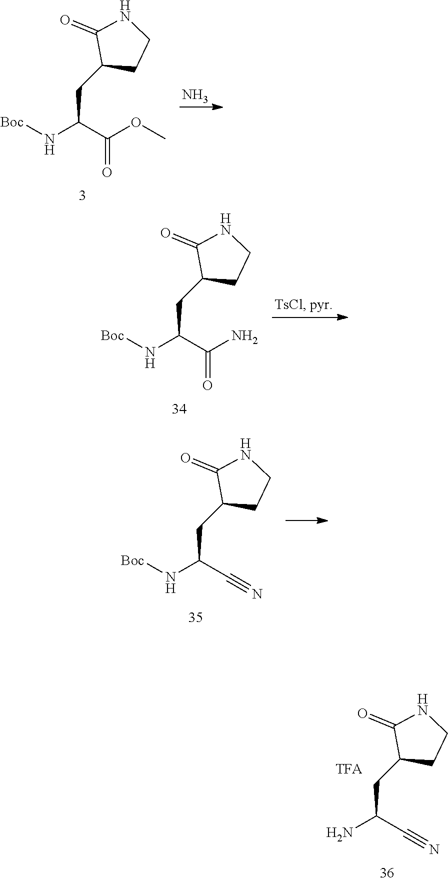

Amidation of 3 with ammonia yields 34. Reaction of 34 with p-toluenesulfonylchloride and pyridine yields the nitrile of 35. Deprotection of 35 with TFA yields 36.

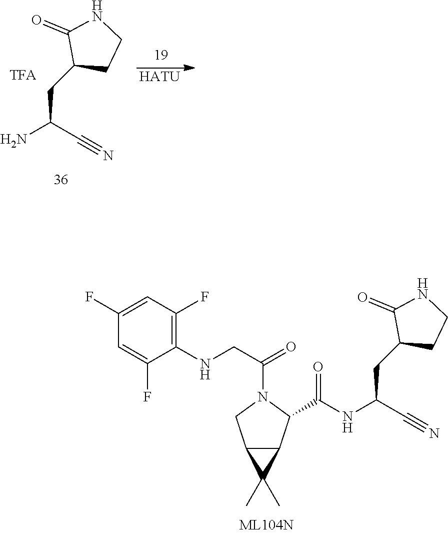

Condensation of 19 with 36 using HATU provides ML104N.

### Synthesis of Alternative R₃ Ketoamides.

The examples above have all used R₃ gamma-lactamyl. However, to those skillful in the art it should be clear that other R₃ groups such as alkyl, substituted alkyl, aryl, substituted aryl, heterocycle, or substituted heterocycle are accessible through the synthesis of analogous of the key intermediate 5. These analogous can be represented as VI:

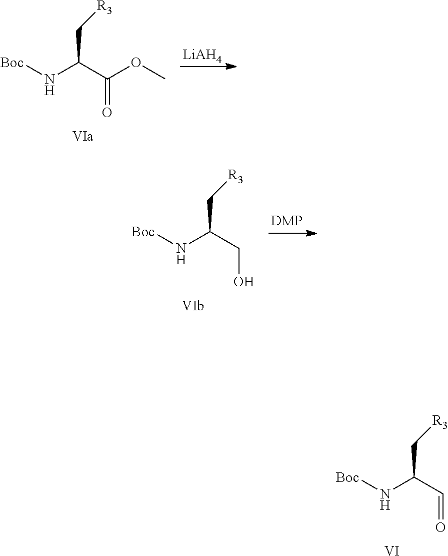

The general route of production of VI, for example begins from the Boc protected amino acid, VIa, or alternatively the corresponding ester. Reduction of VIa with for example LiAlH₄ then yield Vb. VIb is then oxidized using for example Dess-Martin Periodinane to yield VI.

### Confirmation of Final Compounds by Mass Spectroscopy

The following compounds (the structures of which are shown in the figures) were successfully synthesized as confirmed by ES-API LCMS of the protonated compounds. For all compounds, recorded H-NMR spectra were consistent with the expect structure.

| Compound | Expected Mass | Found m/z [M + H]⁺ |
|---|---|---|
| ML1000 | 548.3 | 549.0 |
| ML1100 | 548.3 | 549.1 |
| ML1001 | 547.3 | 548.3 |
| ML1002 | 543.3 | 544.3 |
| ML1002m | 557.3 | 557.8 |
| ML1003 | 533.3 | 534.3 |
| ML101 | 470.2 | 471.2 |
| ML102 | 468.2 | 469.2 |
| ML102m | 482.2 | 482.9 |
| ML104 | 523.2 | 524.2 |
| ML104m | 537.2 | 537.7 |
| ML105 | 509.2 | 510.2 |

### Compositions

The present disclosure also provides compositions. In certain embodiments, the compositions find use, e.g., in practicing the methods of the present disclosure.

According to some embodiments, a composition of the present disclosure includes an compound of the present disclosure. For example, the compound may be any of the protease inhibitor compounds that find use in inhibiting viral protease activity (e.g., coronavirus viral protease activity, such as SARS-CoV-2 protease activity) described in the Compounds section hereinabove or shown in the figures of the present disclosure, which are incorporated but not reiterated herein for purposes of brevity.

In certain aspects, a composition of the present disclosure includes the compound present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, a protease inhibitor, glycerol, a chelating agent, and the like may be present in such compositions.

As summarized above, aspects of the present disclosure include pharmaceutical compositions. In some embodiments, a pharmaceutical composition of the present disclosure includes an effective amount of one or more of any of the compounds of the present disclosure, and a pharmaceutically acceptable carrier.

As will be appreciated, the pharmaceutical compositions of the present disclosure may include any of the compounds and features described herein in the Compounds and Methods of Use sections, which are incorporated but not reiterated in detail herein for purposes of brevity.

Any of the pharmaceutical compositions of the present disclosure may comprise a “cocktail” of two or more different anti-viral agents (e.g., anti-coronavirus agents, such as two or more different anti-SARS-CoV-2 agents), where at least one of the agents is a compound of the present disclosure. In certain embodiments, the pharmaceutical composition further comprises a coronavirus polymerase inhibitor, e.g., a SARS-CoV-2 polymerase inhibitor. According to some embodiments, the coronavirus polymerase inhibitor is selected from remdesivir and favipiravir.

The compounds of the present disclosure can be incorporated into a variety of formulations for therapeutic administration. More particularly, a compound of the present disclosure can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the compounds for administration to an individual (e.g., suitable for human administration) are generally sterile and may further be free of detectable pyrogens or other contaminants contraindicated for administration to a patient according to a selected route of administration, including but not limited to, parenteral, inhalational, intranasal, subcutaneous, intramuscular, and/or intravenous administration.

In pharmaceutical dosage forms, the compound can be administered in the form of their pharmaceutically acceptable salts, or they may also be used alone or in appropriate association, as well as in combination, with other pharmaceutically active compounds. The following methods and carriers/excipients are merely examples and are in no way limiting.

For oral preparations, the compound can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

A compound of the present disclosure can be formulated for parenteral (e.g., intravenous, intra-arterial, intraosseous, intramuscular, intracerebral, intracerebroventricular, intrathecal, subcutaneous, etc.) administration. In certain aspects, the compound is formulated for injection by dissolving, suspending or emulsifying the compound in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include a compound of the present disclosure may be prepared by mixing the compound having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of a compound of the present disclosure may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the compound against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, the pharmaceutical composition includes a compound of the present disclosure, and one or more of the above-identified components (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Kits

Also provided by the present disclosure are kits. The kits find use, e.g., in practicing the methods of the present disclosure. In some embodiments, a subject kit includes a composition (e.g., a pharmaceutical composition) that includes any of the compounds of the present disclosure. In some embodiments, provided are kits that include any of the pharmaceutical compositions described herein, including any of the pharmaceutical compositions described above in the section relating to the compositions of the present disclosure. Kits of the present disclosure may include instructions for administering the pharmaceutical composition to an individual in need thereof, including but not limited to, an individual having or suspected of having a SARS-COV-2 infection, e.g., COVID-19.

The subject kits may include a quantity of the compositions, present in unit dosages, e.g., ampoules, or a multi-dosage format. As such, in certain embodiments, the kits may include one or more (e.g., two or more) unit dosages (e.g., ampoules) of a composition comprising any of the compounds of the present disclosure. The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular compound employed, the effect to be achieved, and the pharmacodynamics associated with the compound, in the individual. In yet other embodiments, the kits may include a single multi dosage amount of the composition.

As will be appreciated, the kits of the present disclosure may include any of the compounds and features described elsewhere herein in the sections relating to the subject compounds, methods and compositions, which are not reiterated in detail herein for purposes of brevity.

Components of the kits may be present in separate containers, or multiple components may be present in a single container. A suitable container includes a single tube (e.g., vial), ampoule, one or more wells of a plate (e.g., a 96-well plate, a 384-well plate, etc.), or the like.

The instructions (e.g., instructions for use (IFU)) included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

### Methods of Use

Aspects of the present disclosure include methods comprising administering a compound of the present disclosure to an individual in need thereof, e.g., an individual having or suspected of having a coronavirus infection (e.g., a SARS-CoV-2 infection). In certain embodiments, provided are methods of treating or preventing a coronavirus infection in an individual, the method comprising administering to the individual a pharmaceutical composition comprising a therapeutically effective amount of any of the compounds of the present disclosure. In certain embodiments, the method is for treating or preventing a SARS-CoV-2 infection in the individual.

The pharmaceutical composition may be administered to any of a variety of individuals. In certain aspects, the individual is a “mammal” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the individual is a human. In certain embodiments, the individual is an animal model (e.g., a mouse model, a primate model, or the like) of a SARS-CoV-2 infection, e.g., an animal model of COVID-19.

The compound is administered in a therapeutically effective amount. By “therapeutically effective amount” is meant a dosage sufficient to produce a desired result, e.g., an amount sufficient to effect beneficial or desired therapeutic (including preventative) results, such as a reduction in a symptom of a SARS-COV-2 infection (e.g., a symptom of COVID-19), as compared to a control. In some embodiments, the therapeutically effective amount is sufficient to slow the progression of, or reduce, one or more symptoms of a SARS-COV-2 infection (e.g., one or more COVID-19 symptoms) selected from viral load, hypoxia (e.g., oxygen saturation levels below 95%, e.g., as measured by pulse oximetry), pneumonia, acute respiratory distress syndrome, thrombosis in the pulmonary microcirculation, and/or the like. According to some embodiments, the therapeutically effective amount slows the progression of, or reduces, one or more of such symptoms by 10% or more, 20% or more, 30% or more, 40% or more, 50% or more, 60% or more, 70% or more, 80% or more, 90% or more, or 100% or more, as compared to the one or more symptoms in the absence of the administration of the compound. An effective amount can be administered in one or more administrations.

When the methods include administering a combination of a compound of the present disclosure and a second agent (e.g., a second agent approved for treatment of a SARS-COV-2 infection, e.g., COVID-19, a non-limiting example of which is a SARS-CoV-2 polymerase inhibitor, e.g., remdesivir), the compound and the second agent may be administered concurrently (e.g., in the same or separate formulations), sequentially, or both. For example, according to certain embodiments, the second agent is administered to the individual prior to administration of the compound, concurrently with administration of the compound, or both. In some embodiments, the compound is administered to the individual prior to administration of the second agent, concurrently with administration of the second agent, or both.

In some embodiments, the one or more agents are administered according to a dosing regimen approved for individual use. In some embodiments, the administration of the compound permits the second agent to be administered according to a dosing regimen that involves one or more lower and/or less frequent doses, and/or a reduced number of cycles as compared with that utilized when the second agent is administered without administration of the compound. In some embodiments, the administration of the second agent permits the compound to be administered according to a dosing regimen that involves one or more lower and/or less frequent doses, and/or a reduced number of cycles as compared with that utilized when the compound is administered without administration of the second agent.

Desired relative dosing regimens for agents administered in combination may be assessed or determined empirically, for example using ex vivo, in vivo and/or in vitro models; in some embodiments, such assessment or empirical determination is made in vivo, in a patient population (e.g., so that a correlation is established), or alternatively in a particular subject of interest.

A compound of the present disclosure, and if also administered, a second agent, may be administered via a route of administration independently selected from oral, parenteral (e.g., by intravenous, intra-arterial, subcutaneous, intramuscular, or epidural injection), inhalational, or intranasal administration.

As described above, aspects of the present disclosure include methods for treating an individual having or suspected of having a coronavirus (e.g., SARS-CoV-2) infection, e.g., COVID-19. By treatment is meant at least an amelioration of one or more symptoms associated with the coronavirus (e.g., SARS-CoV-2) infection (e.g., COVID-19) of the individual, where amelioration is used in a broad sense to refer to at least a reduction in the magnitude of a parameter, e.g., symptom, associated with the coronavirus infection. Non-limiting examples of such symptoms include one or more of viral load, hypoxia (e.g., oxygen saturation levels below 95%, e.g., as measured by pulse oximetry), pneumonia, acute respiratory distress syndrome, thrombosis in the pulmonary microcirculation, and/or the like. As such, treatment also includes situations where the coronavirus infection, or at least one or more symptoms associated therewith, are completely inhibited, e.g., prevented from happening, or stopped, e.g., terminated, such that the individual no longer suffers from the coronavirus infection, or at least the symptoms that characterize the coronavirus infection.

Aspects of the present disclosure further include methods of treating or preventing a SARS-CoV-2 infection in an individual, the method comprising administering to the individual a pharmaceutical composition comprising boceprevir (BPV), narlaprevir (NPV), telaprevir (TPV), rupintrivir, or any combination thereof, in an amount effective to treat or prevent a SARS-CoV-2 infection in the individual.

### Methods of Assessing Inhibition of Protease Activity

Also provided by the present disclosure are methods of assessing inhibition of protease activity.

In some aspects, provided are methods of assessing inhibition of coronavirus protease activity by an agent, the method comprising culturing a cell comprising a first nucleic acid sequence that encodes a coronavirus main protease (Mpro) and a second nucleic acid sequence that encodes a fusion protein comprising a substrate for the Mpro disposed between an optically detectable protein and a membrane localization signal. The culturing is under conditions in which the Mpro and fusion protein are expressed and the fusion protein is localized to the cell membrane via the membrane localization signal. Such methods further comprise introducing an agent into the cell, and assessing cellular localization of the optically detectable protein, wherein retention of cell membrane localization of the optically detectable protein indicates that the agent is an inhibitor of the Mpro. According to some embodiments, the Mpro is SARS-CoV-2 Mpro. In certain embodiments, the substrate for the Mpro comprises the amino acid sequence SAVLQ↓SGFRK (SEQ ID NO:1), TSAVLQ↓SGFRK (SEQ ID NO:2) and/or VTFQ↓SAVKRTIKGTTS (SEQ ID NO:3).

According to some embodiments, the optically detectable protein is a fluorescent protein. Non-limiting examples of such proteins include green fluorescent protein (GFP), a blue fluorescent protein (BFP), a cyan fluorescent protein (CFP), a yellow fluorescent protein (YFP), an orange fluorescent protein (OFP), and red fluorescent protein (RFP). In certain embodiments, the optically detectable protein is a luminescent protein, e.g., a luciferase. According to some embodiments, the cell is a human cell.

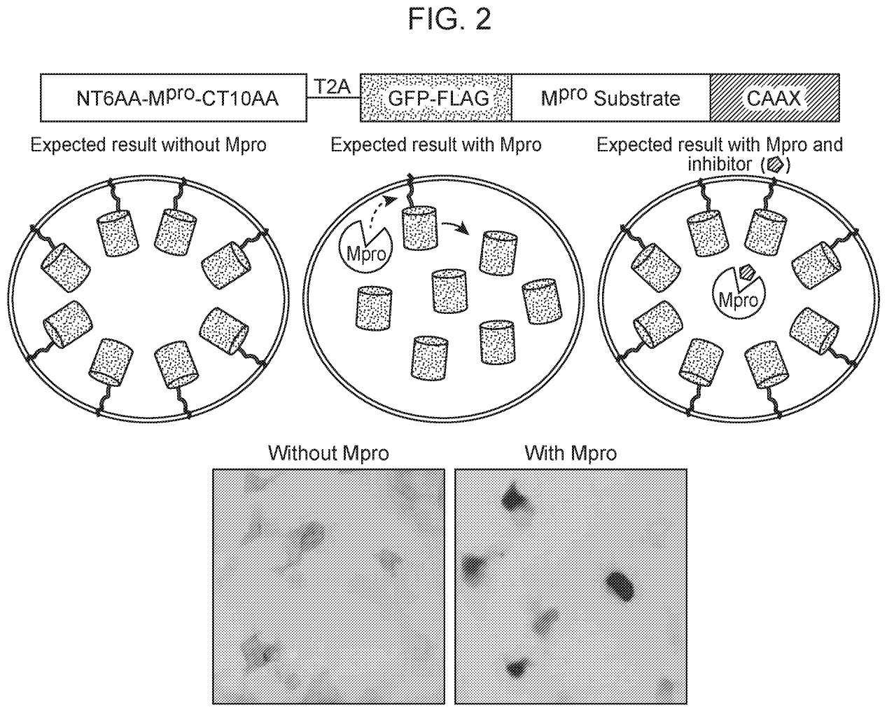

A non-limiting example of a method of assessing inhibition of coronavirus protease activity by an agent according to the above aspect is schematically illustrated in FIG. 2.

In certain aspects, provided are methods of assessing inhibition of activity of a protease by an agent, the methods comprising culturing a cell (e.g., a human cell) comprising a nucleic acid sequence that encodes a reporter polypeptide. The reporter polypeptide comprises, in order: a first portion of a split reporter protein, a first flexible linker comprising a substrate for a protease, the protease, a second flexible linker comprising a substrate for the protease, and a remaining portion of the split reporter protein. The culturing is under conditions in which the cell expresses the reporter polypeptide and the protease cleaves one or both of the flexible linkers in the absence of inhibition of activity of the protease, thereby inactivating the split reporter protein by separating the first and remaining portions thereof. Such methods further comprise introducing an agent into the cell, and assaying for activity of the reporter protein to assess inhibition of activity of the protease by the agent, wherein activity of the reporter protein indicates inhibition of activity of the protease by the agent. According to some embodiments, the protease is a coronavirus main protease (Mpro). In certain embodiments, the Mpro is SARS-CoV-2 Mpro.

According to some embodiments, the first flexible linker and the second flexible linker comprise a substrate for the SARS-CoV-2 Mpro independently selected from a substrate comprising the amino acid sequence SAVLQ↓SGFRK (SEQ ID NO:1), a substrate comprising the amino acid sequence TSAVLQ↓SGFRK (SEQ ID NO:2) and a substrate comprising the amino acid sequence VTFQ↓SAVKRTIKGTTS (SEQ ID NO:3).

In certain embodiments, the split reporter protein is a split luminescent protein. For example, the split reporter protein may be a split luciferase. A non-limiting example of a split luciferase which may be employed is the split NanoLuc luciferase, NanoBit, which comprises portions SmBiT and LgBit.

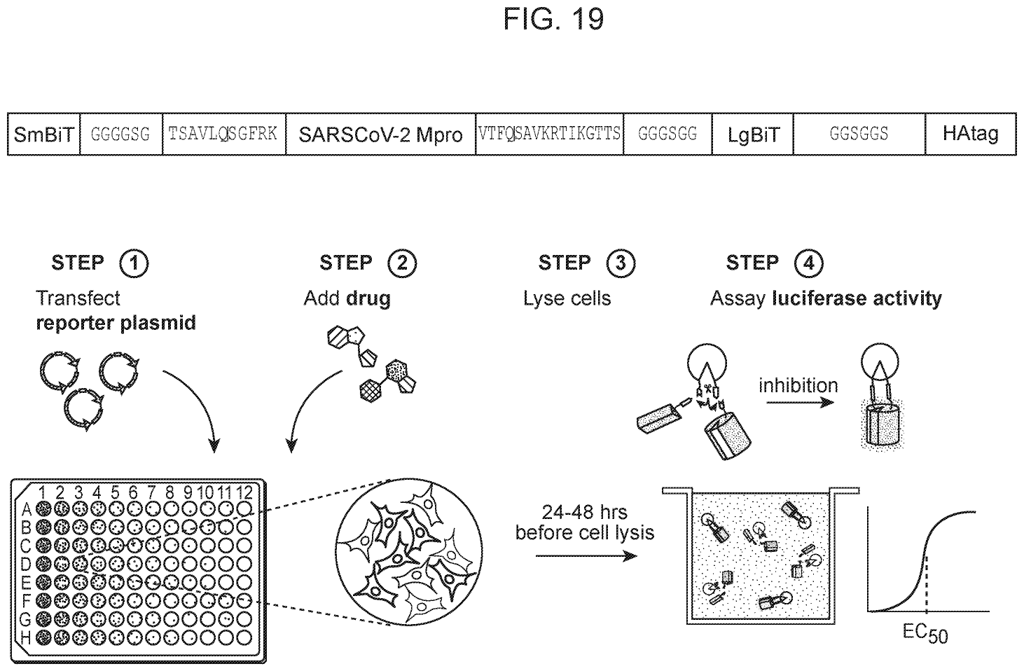

A non-limiting example of a method of assessing inhibition of activity of a protease by an agent using split reporter protein is provided in Example 5 hereinbelow and schematically illustrated in FIG. 19.

Also provided by the present disclosure are reporter polypeptides comprising, in order: a first portion of a split reporter protein, a first flexible linker comprising a substrate for a protease, the protease, a second flexible linker comprising a substrate for the protease, and a remaining portion of the split reporter protein. Nucleic acids encoding such reporter polypeptides are also provided, as are cells (e.g., human cells) comprising such reporter polypeptides and/or nucleic acids. Kits comprising such reporter polypeptides nucleic acids and/or cells are also provided. Such kits may further include instructions for assessing inhibition of activity of a protease by an agent using the components of the kit.

According to any of the assessment methods of the present disclosure, the agent may be a small molecule. By “small molecule” is meant a compound having a molecular weight of 1000 atomic mass units (amu) or less. In some embodiments, the small molecule is 750 amu or less, 500 amu or less, 400 amu or less, 300 amu or less, or 200 amu or less. In certain embodiments, the small molecule is not made of repeating molecular units such as are present in a polymer.

The following examples are offered by way of illustration and not by way of limitation.

### EXPERIMENTAL

### Example 1—New Use of Boceprevir, Narlaprevir, Telaprevir, and Rupintrivir in Treating or Preventing Coronavirus Disease

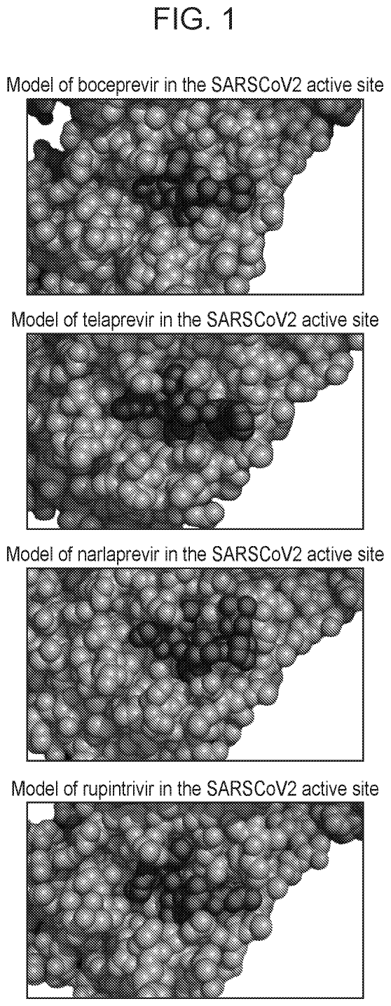

Identified in the structures of SARS-CoV-2 and other coronavirus M proteases was the presence of a large hydrophobic S2 pocket (3, 4). S2 is the part of the enzyme active site that binds the side chain of the P2 residue, which is the second residue N-terminal to the cleavage site. Based on our work with hepatitis C virus (HCV) and human rhinovirus (HRV) proteases, it was understood that HCV and HRV inhibitors contain large proline-based rings in the P2-analogous location. Provided for the first time herein is that the central proline-based ring structure of BPV, TPV, and NPV can bind to the S2 pocket of coronavirus protease M (Mpro), even though neither Mpro substrates nor purpose-made coronavirus Mpro inhibitors have a proline at P2. In addition, while rupintrivir has been found to have no activity against the SARS-CoV-1 virus, which is closely related to SARS-CoV-2 (5), it was hypothesized that SARS-CoV-2 protease could have more structural flexibility than SARS-CoV-1 protease and thus accommodate rupintrivir better. The docking of BPV and TPV was also predicted computationally by other groups (6, 7), but NPV and rupintrivir have not been predicted by others. Docking of HCV and HRV inhibitors to the structures of SARS-CoV-2 and other coronavirus M proteases was performed, and it was found that the HCV inhibitors boceprevir (BPV), narlaprevir (NPV), and telaprevir (TPV) fit inside the substrate binding pocket (FIG. 1). The HRV acceptor rupintrivir can also fit (FIG. 1).

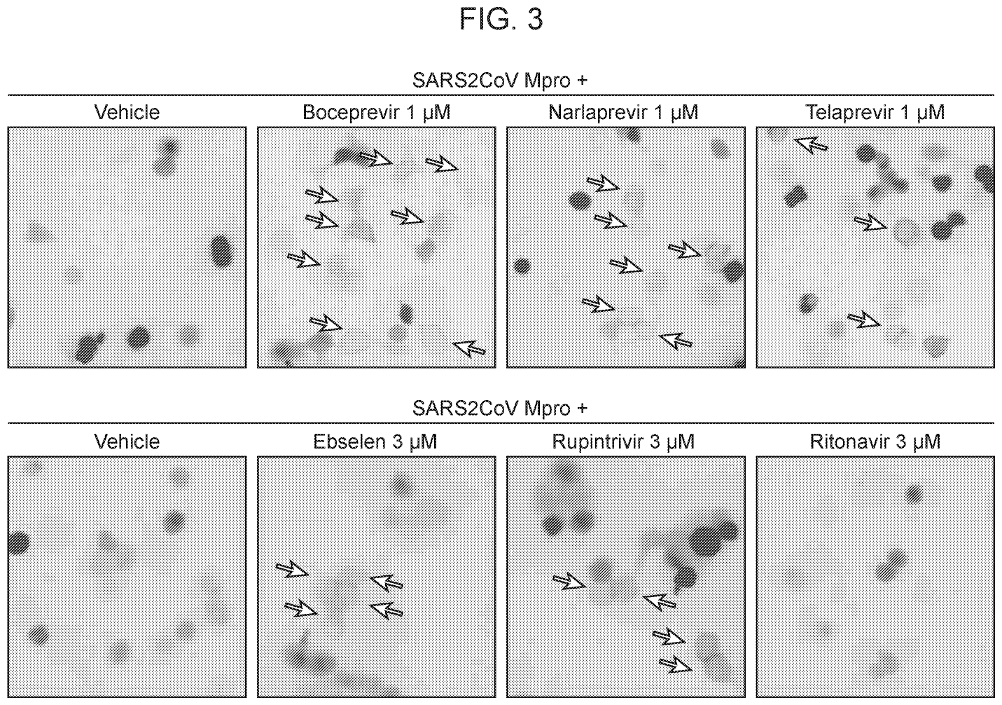

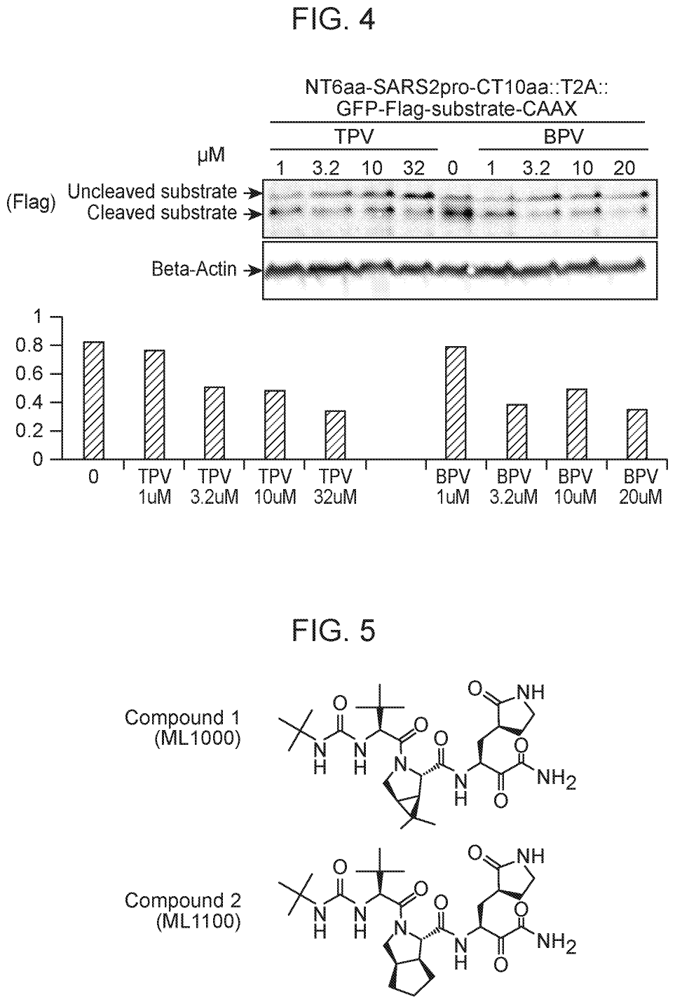

SARS-CoV-2 Mpro was expressed with native N and C termini to allow its self-processing. To characterize protease activity and inhibitor potency, a cell-based trans-cleavage translocation assay was developed, where the Mpro substrate SAVLQ↓SGFRK (SEQ ID NO:1), based on the N-terminal auto-cleavage site sequence of SARS-CoV2, was placed between GFP and a membrane localization signal (CAAX peptide from human H-Ras protein). Without protease inhibitor administration, GFP is released from the cell membrane whereas, in the presence of Mpro inhibitors, GFP remains membrane-localized (FIG. 2). Both SARS-CoV2 Mpro and its substrate are expressed together in cultured human cells with a single bicistronic vector with a T2A (thosea asigna virus 2A) sequence in between to ensure co-expression of the substrate and the protease. The construct is transfected into cells by Lipofectamine 3000, then potential protease inhibitors are added 2 hours post-transfection. Results in HEK293 cells show that BPV, NPV, TPV, and rupintrivir could effectively inhibit Mpro enzymatic activity. Based on images (FIG. 3) and immunoblots (FIG. 4), EC50 (concentration required for 50% effect of protease activity) was estimated between 1 and 3.2 μM for each of these compounds.

These 4 compounds, BPV, NPV, TPV, and rupintrivir, are thus unexpected inhibitors of SARS-CoV-2 Mpro in human cells. A recent study posted online found BPV and NPV to inhibit a tagged non-native form of SARS-CoV-2 Mpro in vitro and to inhibit SARS-CoV-2 viral replication in monkey Vero cells, but did not perform any tests in human cells (8). The posted findings of low effect by TPV and no effect of rupintrivir are also different from the results of the present study. As inhibitor effectiveness can be different in Vero cells than other cells (9), it is essential to demonstrate efficacy of compounds in human cells.

### Example 2—New Route of Administration for Boceprevir, Narlaprevir, Telaprevir, and Rupintrivir in Treating or Preventing Coronavirus Disease

While BPV, NPV, and TPV were originally designed for oral dosing, and rupintrivir for intranasal dosing, it is hypothesized that intravenous or aerosol formulations would be superior for treatment of COVID19 in the hospital setting. This avoids first-pass metabolism in the liver after oral dosing and trapping in the upper respiratory system in intranasal dosing. It is advantageous to achieve peak concentrations several fold higher than the EC50 for sustained antiviral effect throughout the day. BPV reaches peak plasma concentrations of 3 μM after a maximal 800 mg oral dose and TPV reaches 5 μM after a maximal 750 mg dose (10, 11). These concentrations are near the EC50 values observed herein and thus not high enough to inhibit viral replication continuously. Rupintrivir likewise reaches concentrations on nasal surfaces of 5 μM after a nasal dose (12), but lung concentrations have not been assessed. The use of IV injection or aerosolized preparations for lung delivery for both these drugs will likely improve clinical efficacy.

### REFERENCES

1. www.niaid.nih.gov/news-events/nih-clinical-trial-shows-remdesivir-accelerates-recovery-advanced-covid-19,
2. Y. Wang et al., Remdesivir in adults with severe COVID-19: a randomised, double-blind, placebo-controlled, multicentre trial. The Lancet online first, (2020).
3. Z. Jin et al., Structure of Mpro from COVID-19 virus and discovery of its inhibitors. Nature (2020).
4. L. Zhang et al., Crystal structure of SARS-CoV-2 main protease provides a basis for design of improved α-ketoamide inhibitors. Science 368, 409-412 (2020).
5. C. Y. Wu et al., Small molecules targeting severe acute respiratory syndrome human coronavirus. Proc. Natl. Acad. Sci. U.S.A 101, 10012-10017 (2004).
6. K. Bagherzadeh, K. Daneshvarnejad, M. Abbasinazari, H. azizian, In silico Repositioning for Dual Inhibitor Discovery of SARS-CoV-2 (COVID-19) 3C-like Protease and Papain-like Peptidase. (2020).
7. D. D. Nguyen, K. Gao, J. Chen, R. Wang, G.-W. Wei, Potentially highly potent drugs for 2019-nCoV. bioRxiv 2020.02.05.936013 (2020).
8. C. Ma et al., Boceprevir, GC-376, and calpain inhibitors II, XII inhibit SARS-CoV-2 viral replication by targeting the viral main protease. bioRxiv 2020.04.20.051581 (2020).
9. L. Zhang et al., α-Ketoamides as Broad-Spectrum Inhibitors of Coronavirus and Enterovirus Replication: Structure-Based Design, Synthesis, and Activity Assessment. J. Med. Chem. (2020).
10. FDA, boceprevir prescribing information. www.accessdata.fda.gov/drugsatfda_docs/label/2011/2022581bl.pdf
11. FDA, telaprevir prescribing information. www.accessdata.fda.gov/drugsatfda_docs/label/2011/2019171bl.pdf
12. R.-W. Helga, D. Karl, H. Guy, W. Reinhold, Viral Infections and Treatment. (2003).

### Example 3—New Chemical Entities for Treating or Preventing Coronavirus Disease

In addition, provided herein are new inhibitors based on the inventors' observation that the central ring structure of BPV, TPV, and NPV improves affinity by creating a rigid hydrophobic surface that can insert rapidly into the S2 pocket of SARS-CoV-2 Mpro and those of related coronaviruses, thereby minimizing the entropic penalty of inhibitor conformational rigidification upon binding.

One new compound, compound 1, is based on BPV but uses a gamma-lactamylmethyl group in place of the cyclobutylmethyl group (immediately adjacent to the alpha-ketoamide group). The position of the cyclobutylmethyl group is analogous to that of the P1 side chain in the natural substrate, that is the side-chain immediately N-terminal to the cleavage site. A lactamylmethyl group, by mimicking the hydrogen bonding patterns of the natural glutamine sidechain at P1, may provide more energetically favorable binding to the S1 pocket. (FIG. 5).

Also designed was compound 2 (FIG. 5) based on compound 1 but using a central proline-based ring structure derived TPV.

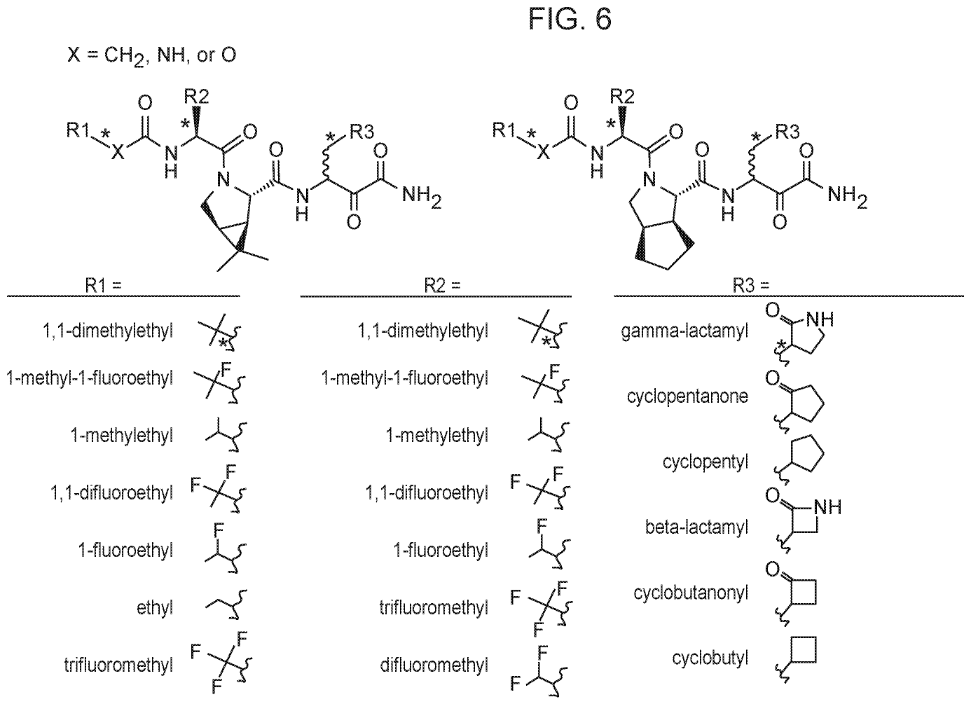

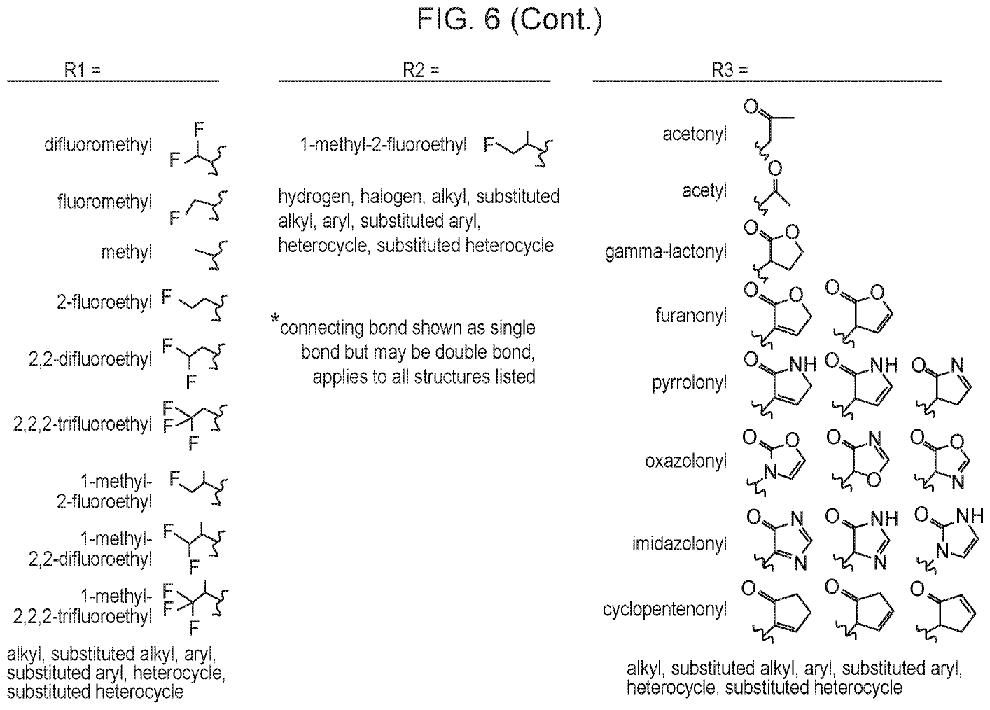

Lastly, further new compounds are envisioned that are higher affinity than compounds 1 and 2 while retaining good drug-like properties. Based on the inventors' modeling, affinity of compounds 1 and 2 are likely limited by the 1,1-dimethylethyl (same as t-butyl) group in the P4 position (attached to the uryl group) being too branched for the S4 pocket. Affinity can be tuned by reducing the sizes of groups attached to the carbon attached directly to the uryl group by substituting the 1,1-dimethylethyl group with 1-methyl-1-fluoroethyl; 1-methylethyl (equivalent to propyl); 1,1-difluoroethyl; 1-fluoroethyl; ethyl; trifluoromethyl; difluoromethyl; fluoromethyl; methyl; 1-methyl-2,2,2-trifluoroethyl; 1-methyl-2,2-difluoroethyl; 1-methyl-2-fluoroethyl; 2,2,2-trifluoroethyl; 2,2-difluoroethyl; 2-fluoroethyl; other alkyl; other substituted alkyl; an aryl; a substituted aryl; a heterocycle; or a substituted heterocycle (FIG. 6). Some of these modifications can also improve desirable pharmacological properties such as cell permeability, bioavailability, chemical stability, resistance to metabolism, or low toxicity. For example, the fluorinated groups above may improve cell membrane penetration and thereby reduce EC50. This list is not meant to be a comprehensive list; other structures that are less branched than t-butyl are familiar to practitioners in the field of chemical synthesis.

In combination with P4 optimization, the 1,1-dimethylethyl group at P3 can also be optimized for better drug-like properties. In natural substrates, the P3 position is occupied by a wide variety of amino acids. It can be changed to 1-methyl-1-fluoroethyl; 1-methylethyl (equivalent to propyl); 1,1-difluoroethyl; 1-fluoroethyl; trifluoromethyl; difluoromethyl; 1-methyl-2,2,2-trifluoroethyl; or 1-methyl-2,2-difluoroethyl; 1-methyl-2-fluoroethyl; other alkyl; other substituted alkyl; an aryl; a substituted aryl; a heterocycle; a substituted heterocycle; halogen; or hydrogen to improve desirable pharmacological properties such as cell permeability, bioavailability, chemical stability, resistance to metabolism, or low toxicity (FIG. 6). This list is not meant to be a comprehensive list; other suitable structures are familiar to practitioners in the field of chemical synthesis.

In combination with P4 and P3 optimizations, the P1 position can also be optimized. The present discovery of inhibition of coronavirus proteases by drugs with both cyclobutyl and gamma-lactamyl groups at P1 (following the methylene bridge) suggests this position can accept a 4- or 5-membered ring with or without an aldehyde. This position thus can be optimized by substituting the gamma-lactamyl group with cyclopentanonyl, cyclopentyl, beta-lactamyl, cyclobutanonyl, cyclobutyl, acetonyl, acetyl, gamma-lactonyl, furanonyl, pyrrolonyl, cyclopentenonyl, oxazolonyl, imidazolonyl, other alkyl, other substituted alkyl, an aryl, a substituted aryl, other heterocycle, or other substituted heterocycle (FIG. 6). This list is not meant to be a comprehensive list; other suitable structures are familiar to practitioners in the field of chemical synthesis.

In addition, the interaction of compounds (having an NH group that serves as the linkage for the P4 group) with the S4 pocket of the protease may be improved by allowing a non-coplanar conformation at the linkage between the P4 group (which is 1,1-dimethylethyl, same as t-butyl) and the uryl group. Affinity can therefore be optimized by substituting the NH group that serves as the linkage for the P4 group with either an oxygen atom or a methylene (CH2) group. This can be done while substituting the 1,1-dimethylethyl group at P4 with other groups that have the proper shape-complementarity with the S4 pocket such as 1-methyl-1-fluoroethyl; 1-methylethyl (equivalent to propyl); 1,1-difluoroethyl; 1-fluoroethyl; ethyl; trifluoromethyl; difluoromethyl; fluoromethyl; methyl; 1-methyl-2,2,2-trifluoroethyl; 1-methyl-2,2-difluoroethyl; 1-methyl-2-fluoroethyl; 2,2,2-trifluoroethyl; 2,2-difluoroethyl; 2-fluoroethyl; other alkyl; other substituted alkyl; an aryl; a substituted aryl; a heterocycle; or a substituted heterocycle (FIG. 6). Some of these modifications can also improve desirable pharmacological properties such as cell permeability, bioavailability, chemical stability, resistance to metabolism, or low toxicity. For example, the fluorinated groups above may improve cell membrane penetration and thereby reduce EC50. This list is not meant to be a comprehensive list; other structures are familiar to practitioners in the field of chemical synthesis.

Independent of the optimization of P4 and its linkage, the 1,1-dimethylethyl group at P3 can also be optimized for better drug-like properties. In natural substrates, the P3 position is occupied by a wide variety of amino acids. It can be changed to 1-methyl-1-fluoroethyl; 1-methylethyl (equivalent to propyl); 1,1-difluoroethyl; 1-fluoroethyl; trifluoromethyl; difluoromethyl; 1-methyl-2,2,2-trifluoroethyl; or 1-methyl-2,2-difluoroethyl; 1-methyl-2-fluoroethyl; other alkyl; other substituted alkyl; an aryl; a substituted aryl; a heterocycle; a substituted heterocycle; halogen; or hydrogen to improve desirable pharmacological properties such as cell permeability, bioavailability, chemical stability, resistance to metabolism, or low toxicity (FIG. 6). This list is not meant to be a comprehensive list; other suitable structures are familiar to practitioners in the field of chemical synthesis.

In combination with optimizations of at the P4 group and its linkage, and of P3, the P1 position can also be optimized. The present discovery of inhibition of coronavirus proteases by drugs with both cyclobutyl and gamma-lactamyl groups at P1 (following the methylene bridge) suggests this position can accept a 4- or 5-membered ring with or without an aldehyde. This position thus can be optimized by substituting the gamma-lactamyl group with cyclopentanonyl, cyclopentyl, beta-lactamyl, cyclobutanonyl, cyclobutyl, acetonyl, acetyl, gamma-lactonyl, furanonyl, pyrrolonyl, cyclopentenonyl, oxazolonyl, imidazolonyl, other alkyl, other substituted alkyl, an aryl, a substituted aryl, other heterocycle, or other substituted heterocycle (FIG. 6). This list is not meant to be a comprehensive list; other suitable structures are familiar to practitioners in the field of chemical synthesis.

These additional variants allow optimization of drug properties efficiently, as they can maintain or improve desired pharmacological properties while maintaining or improving affinity to the enzyme. These new chemical entities should be useful for treating diseases caused by SARS-CoV-2 and related coronaviruses.

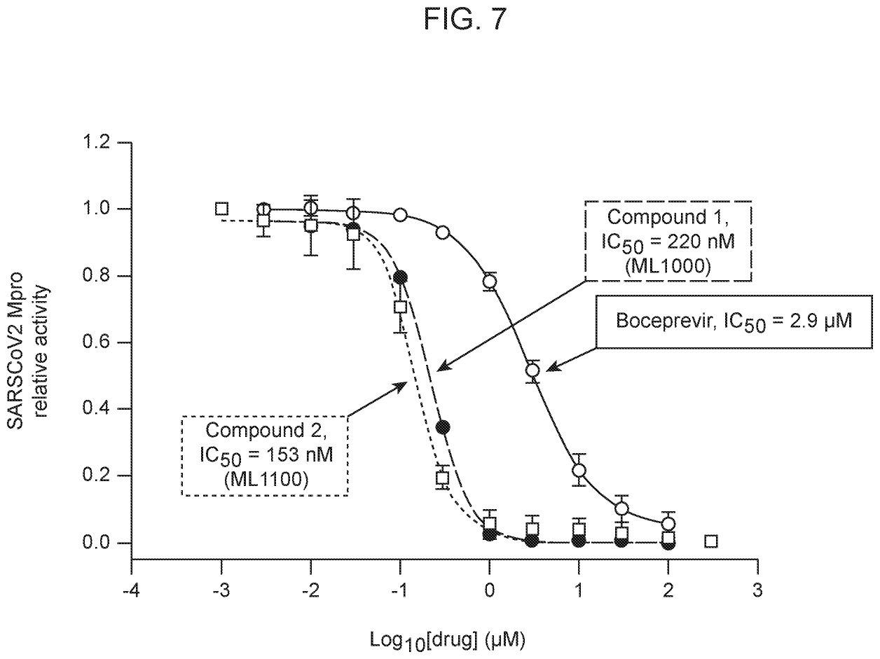

Compound 1 (based on BPV but with a lactonylmethyl group in place of the cyclobutylmethyl group) and Compound 2 (based on compound 1 but using the same central proline-based fused-ring structure as used in TPV) were synthesized and it was determined that Compound 1 indeed inhibits SARS-Co-V2 MPro in vitro with greater potency than BPV (FIG. 7) with concentrations for 50% inhibition (IC50s) of 220 nM for Compound 1 and 2900 nM for BPV, respectively. Compound 2 demonstrated even greater potency, with IC50 of 153 nM.

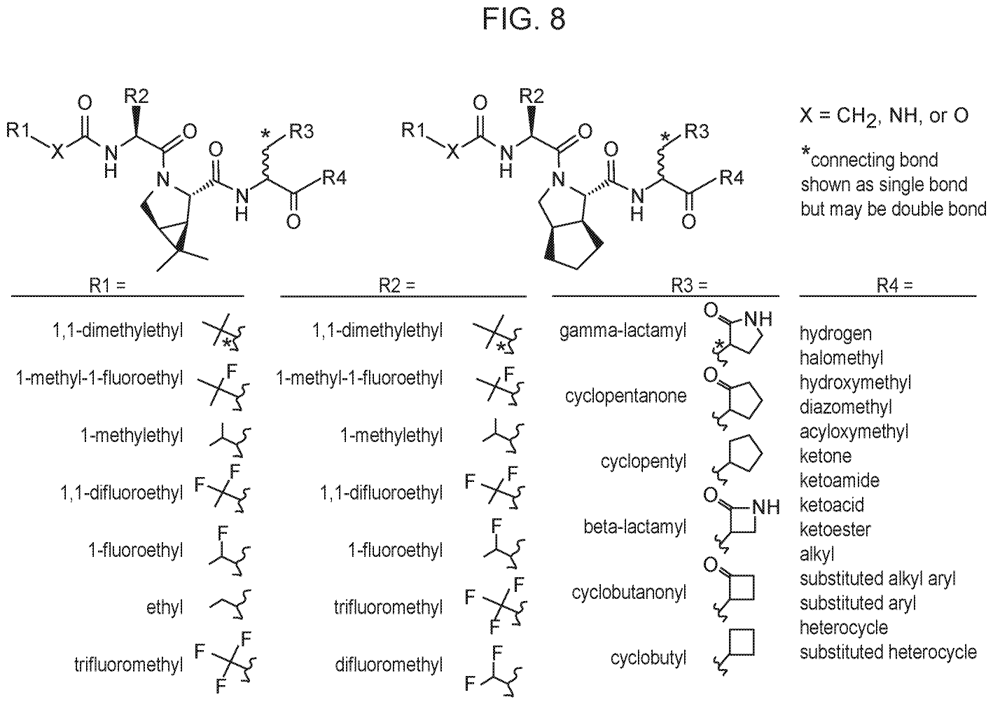

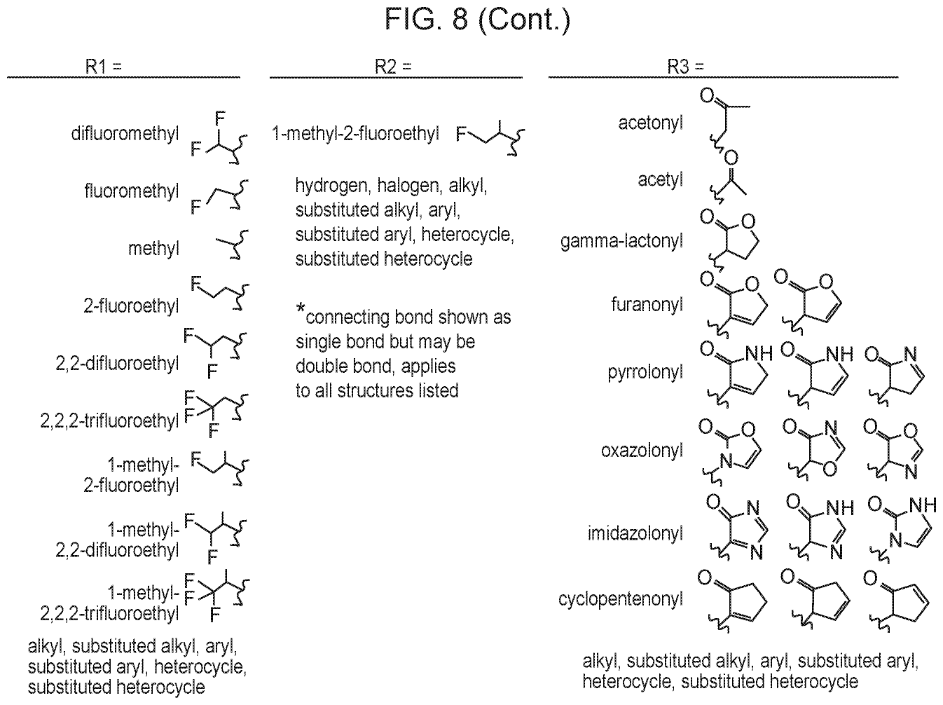

With the goal of increasing permeability and reactivity, the amide group within the ketoamide “warhead” moiety can be replaced with hydrogen (converting the ketoamide to an aldehye), halomethyl, hydroxymethyl, diazomethyl, acyloxymethyl, substituted amide, carboxylic acid, ester, ketone, alkyl, substituted alkyl, aryl, substituted aryl, heterocycle, or substituted heterocycle. This can be combined with the optimizations at P1, P3, and P4 described above (FIG. 8).

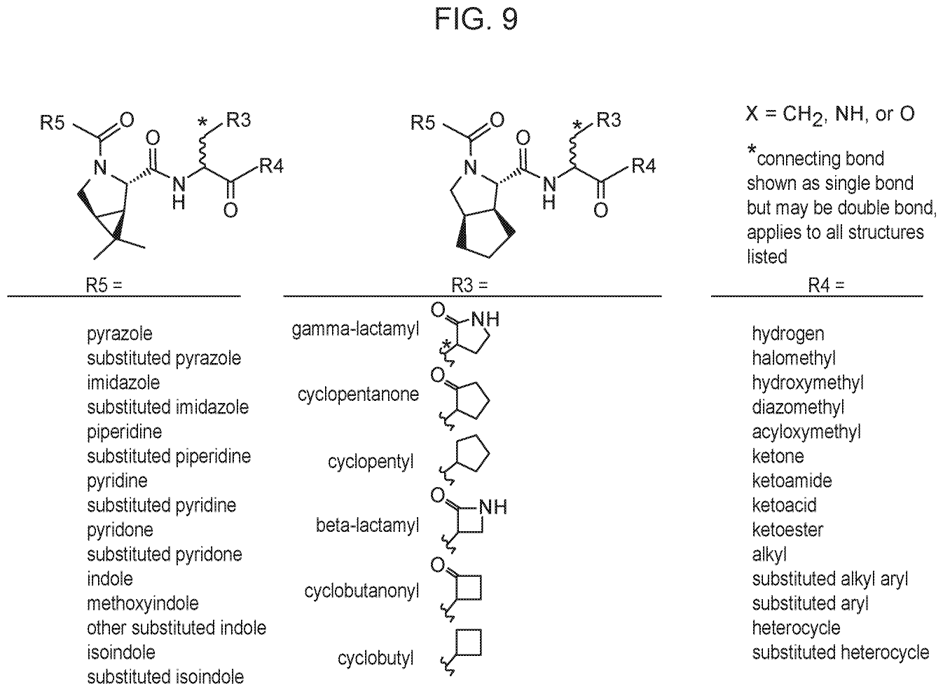

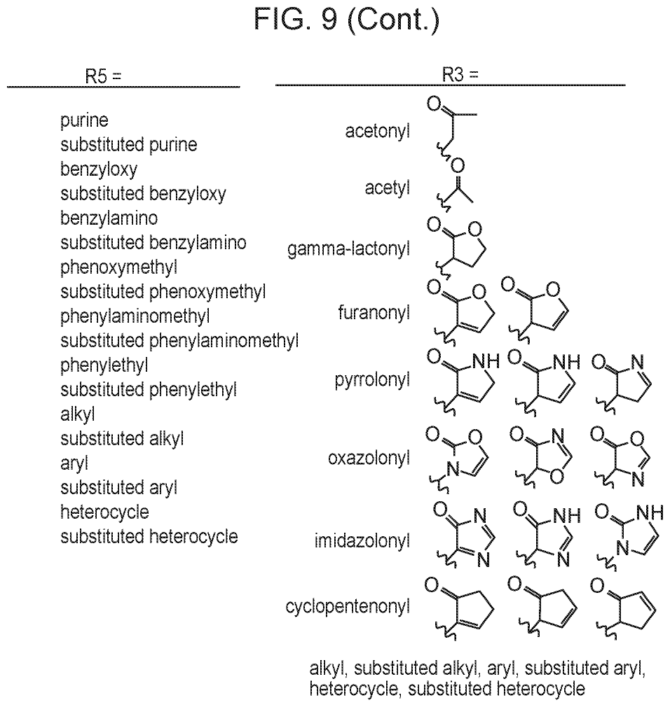

With the goal of increasing permeability while maintaining affinity, P4 sidechain atoms, P4 backbone atoms, P3 sidechain atoms, and the P3 nitrogen and alpha carbon atoms can be eliminated, and in their place can be attached one of the following groups, designed to fill the S4 pocket or the active site groove outside S4: pyrazole, substituted pyrazole, imidazole, substituted imidazole, piperidine, substituted piperidine, pyridine, substituted pyridine, pyridone, substituted pyridone, indole, methoxyindole, other substituted indole, isoindole, substituted isoindole, purine, substituted purine, benzyloxy, substituted benzyloxy, benzylamino, substituted benzylamino, phenoxymethyl, substituted phenoxymethyl, phenylaminomethyl, substituted phenylaminomethyl, phenylethyl, substituted phenylethyl, alkyl, substituted alkyl, aryl, substituted aryl, heterocycle, substituted heterocycle. This can be combined with the optimizations at P1 and the warhead described above (FIG. 9).

### Example 4—Ketoamide Protease Inhibitors Suppress SARS-CoV-2 Replication in Human Cells

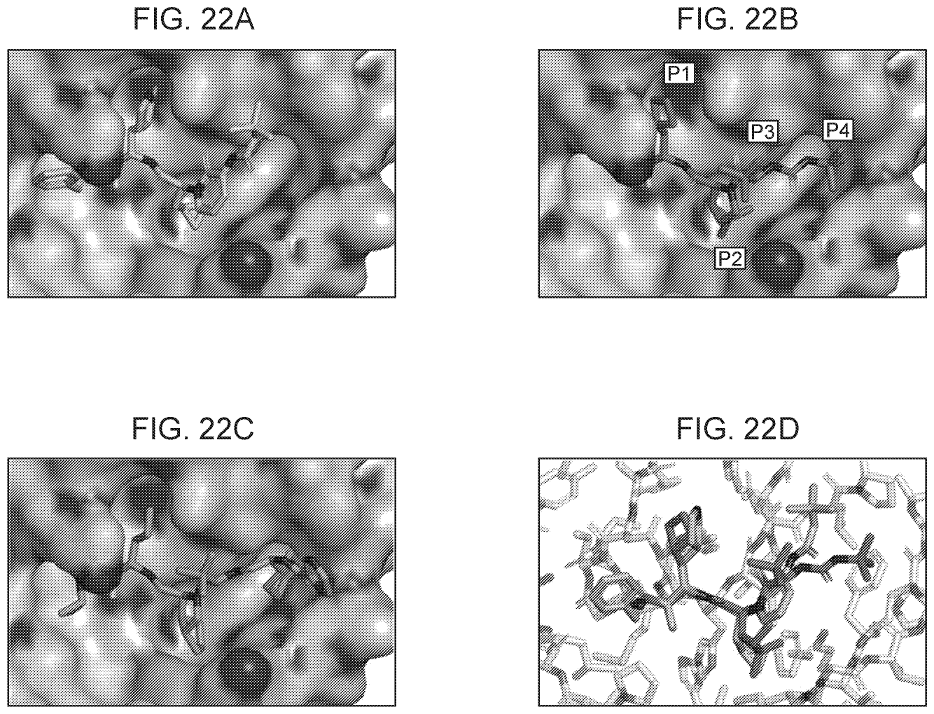

While visualizing the co-crystal structure of the SARS-CoV-2 inhibitor 13b (L. Zhang et al., Crystal structure of SARS-CoV-2 main protease provides a basis for design of improved α-ketoamide inhibitors. Science 368, 409-412 (2020)) and SARS-CoV-2 M^{pro}, it was noticed that 13b adopts a pronounced kink in its main chain at the P2 residue, i.e., the 2^{nd} residue N-terminal to the scissile bond facing the enzyme's S2 pocket (FIG. 22, panel A). This kink was recognized to be similar to that created by proline analog rings in the clinically approved anti-HCV drugs boceprevir, narlaprevir, and telaprevir (FIG. 22, panels B-D). Like 13b, these drugs are ketoamide-based covalent inhibitors, serving as substrates for nucleophilic attack by the deprotonated active-site cysteine.

Manual rigid docking of boceprevir showed that it could fit into the active site of SARS-CoV-2 M^{pro} with good shape complementarity by its P1, P2, and P4 groups (FIG. 22, panel B). In addition, the urea group of boceprevir at the P3-P4 junction could engage in a bidentate hydrogen bond with the backbone carbonyl of M^{pro} Glu-166. The boceprevir derivative narlaprevir also demonstrated complementary in its P1 and P2 groups, which are identical to boceprevir, but its P4 group was clearly too large for the S4 pocket. Telaprevir could also be manually docked with good complementarity of its P1 sidechain and of its P2 group, which is a different proline analog from that of boceprevir (FIG. 22, panel C), whereas its P4 group appeared slightly too large for the S4 pocket. The P3 group of coronavirus M^{pro} substrates face out into solution, as does the t-butyl group in the analogous position of boceprevir, telaprevir, and narlaprevir. Interestingly, the cp and tp angles of the proline ring in boceprevir precisely retraced the backbone atoms of 13b in the bound configuration (FIG. 22, panel D). It was thus hypothesized that boceprevir and, to a lesser extent, narlaprevir and telaprevir, may be able to inhibit SARSCoV2 M^{pro}.

To test this hypothesis, a preliminary rapid assessment was performed using a live-cell assay of M^{pro} function. Co-expressed were SARSCoV2 M^{pro} and a substrate protein comprising green fluorescent protein (GFP), a substrate site, and the membrane-targeting CAAX sequence. Cleavage at the substrate sequence liberates GFP, allowing quantitation of activity by immunoblotting. This permitted assessment of the ability of drugs to inhibit M^{pro} activity in cells. In HEK293A cells, boceprevir and telaprevir were found to inhibit SARSCoV2 M^{pro} at micromolar concentrations (FIG. 4). These results provided evidence of the ability of HCV inhibitors with a P2 proline group to inhibit SARSCoV2 M^{pro}.

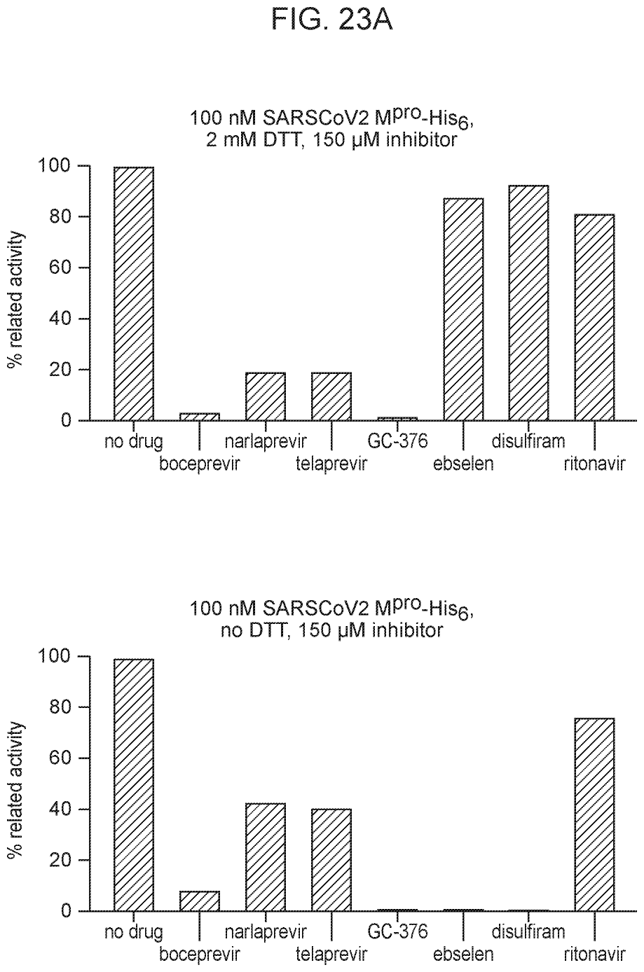

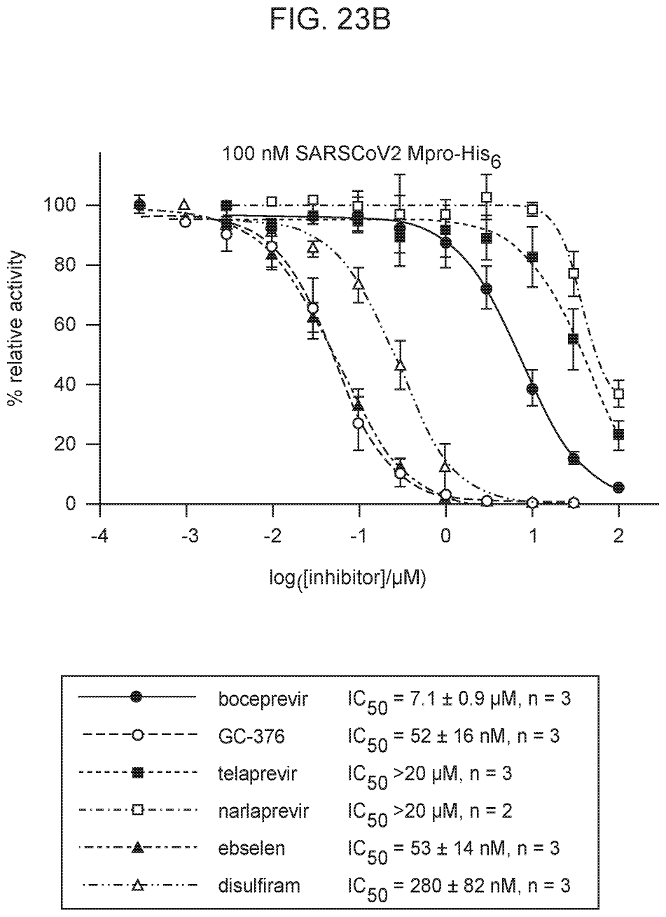

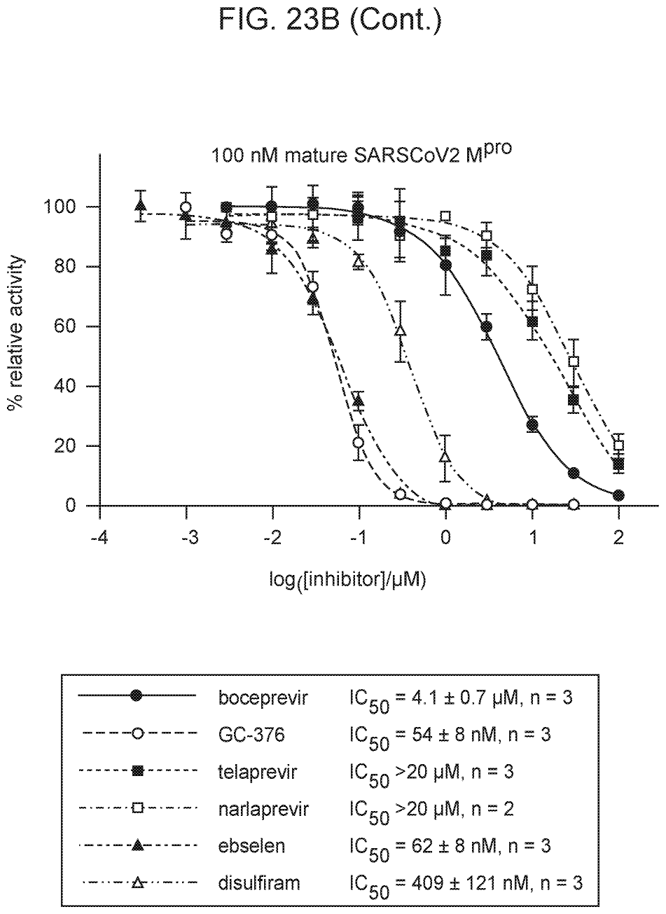

Performed next was a pilot test of the ability of HCV inhibitors with a P2 proline group to inhibit SARSCoV2 M^{pro} in vitro (FIG. 23, panel A). As controls, also tested were GC-376 as a known broad-spectrum coronavirus M^{pro} inhibitor, ebselen and disulfiram as compounds recently reported to inhibit SARSCoV2 M^{pro}, and ritonavir as a protease inhibitor without any structural homology to coronavirus M^{pro} substrates. Indeed, it was observed that boceprevir, narlaprevir, telaprevir, GC-376, disulfiram, and ebselen (at 150 μM concentration in air-equilibrated buffer) were able to inhibit SARSCoV2 M^{pro}, whereas ritonavir showed no inhibitory effect (FIG. 23, panel A). Incidentally, disulfiram and ebselen showed no inhibitory effect in the presence of DTT, whereas other compounds were unaffected by DTT. These results are consistent with proposals of disulfiram and ebselen forming a reducible bond with M^{pro}. As the interior of the cell is reducing, reduction of ebselen adducts could contribute to its relatively high EC₅₀ of 4.7 μM for blocking SARSCoV2 replication in cells.

Full inhibition curves SARSCoV2 M^{pro} were then performed to obtain IC₅₀ values for the above compounds. The initial pilot test above was performed with SARSCoV2 M^{pro} with a C-terminal His₆-tag for rapid purification, but the presence of a C-terminal extension may have an inhibitory effect on protease activity. Inhibition curves were thus performed on both C-terminally extended and fully mature SARSCoV2 M^{pro} (FIG. 23, panel B). Drugs were tested in the presence of DTT, except for ebselen and disulfiram, for which DTT was omitted. The results showed that the proline-containing HCV protease inhibitors boceprevir, telaprevir, and narlaprevir all could inhibit activity to some extent in both C-terminally extended and fully mature forms of SARSCoV2 M^{pro}. Among these, boceprevir was the most potent, with an IC₅₀ value of 4.1 μM on mature SARSCoV2 M^{pro} (FIG. 23, panel B and the table below, where data are presented as mean±standard deviation of 3 replicates. ND, not determined.).

Measured 50% Inhibitory Constants (IC₅₀ Values) of ML1000 (Compound 1) and ML1100 (Compound 2) on SARSCoV2 M^{pro} In Vitro

| | 100 nM mature | 100 nM mature | 20 nM mature | 100 nM mature |
|---|---|---|---|---|
| | SARSCoV2 M^{pro} | SARSCoV2 M^{pro} at | SARSCoV2 M^{pro} | MHV M^{pro} at |
| | at 30° C. (nM) | 37° C. (nM) | at 30° C. (nM) | 30° C. (nM) |
| GC-376 | 54 ± 8 | 53 ± 3 | 14 ± 5 | 67 ± 5 |
| ML1000 | 34 ± 8 | 47 ± 15 | 12 ± 6 | 130 ± 25 |
| ML1100 | 147 ± 8 | 124 ± 36 | ND | 301 ± 152 |
| Boceprevir | 4100 ± 700 | 3700 ± 200 | ND | 15000 ± 7000 |

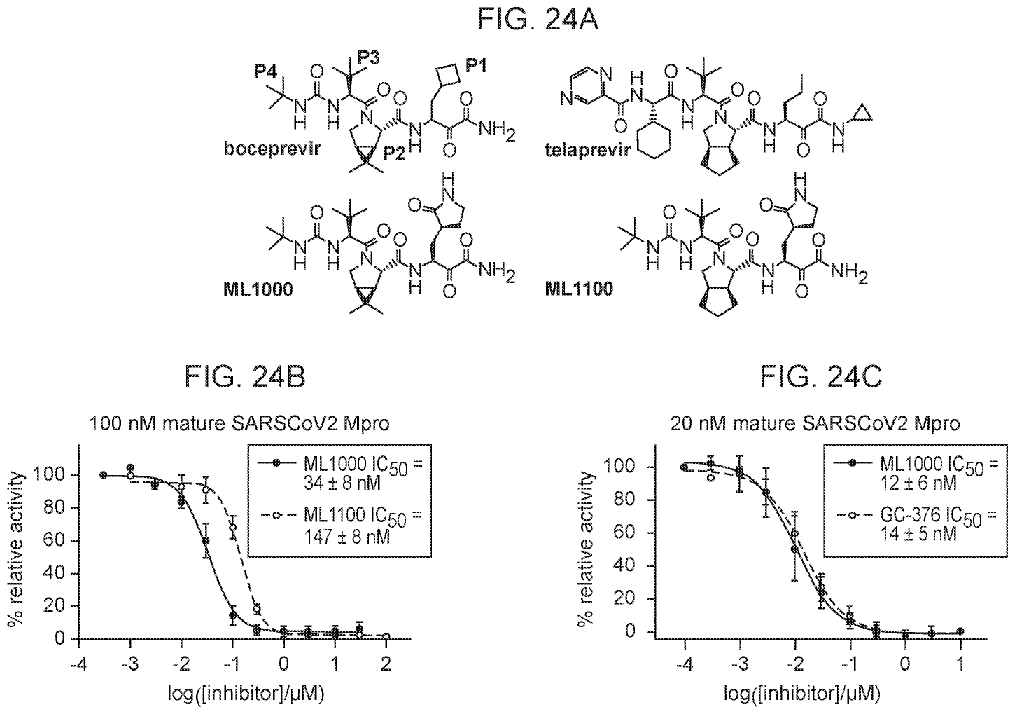

Having established that the P2 proline analogues in boceprevir, telaprevir, and narlaprevir were compatible with binding to the SARSCoV2 M^{pro} active site, next sought was to design next-generation coronavirus inhibitors that could combine the entropic stabilization conferred by these P2 rings with side groups optimized for coronavirus M^{pro} binding. A P1 Gln residue is strongly preferred by all coronavirus M^{pro} species. In fact, this preference is conserved with the related enterovirus 3C proteases such as human rhinovirus (HRV) protease and even with the more distantly related potyvirus proteases such as tobacco etch virus (TEV) protease. Beginning with the HRV protease inhibitors AG7088 (rupintrivir) and its derivative AG7404, enterovirus and coronavirus protease inhibitors have incorporated a γ-lactam group to mimic the hydrogen bond acceptor function of Gln in the P1 position. Enzyme-inhibitor cocrystal structures have confirmed that the γ-lactam amide group is positioned similarly to the Gln amide group in natural structures. Thus, designed was a novel inhibitor, ML1000 (Compound 1, FIG. 5), with the ketoamide warhead of boceprevir, the γ-lactamyl P1 group of rupintrivir, and the P2 through P4 moieties (including backbone and side chains) of boceprevir (FIG. 24, panel A). Also designed was a second inhibitor, ML1100 (Compound 2, FIG. 5), composed of the ketoamide warhead of boceprevir, the γ-lactamyl P1 group of rupintrivir, the P2 cyclic structure of telaprevir, and the P3 and P4 moieties of boceprevir (FIG. 24, panel A). The P3 and P4 structure of boceprevir was retained as these groups did not demonstrate any clashes with the enzyme pocket in the manually docked boceprevir model, and as their hydrophobicity likely contributes to membrane permeability.

ML1000 and ML1100 proved to be highly potent inhibitors of SARSCoV2 M^{pro}. In the presence of 100 nM of enzyme, ML1000 and ML1100 produced IC₅₀ values of 34 nM and 147 nM (FIG. 24, panel B), compared to 54 nM for GC-376 (FIG. 23, panel B). Against the M^{pro} enzyme from the distantly related coronavirus mouse hepatitis virus (MHV), ML1000 and ML1100 exhibited IC₅₀ values of 130 and 301 nM, respectively, compared to 67 nM for GC-376. These results demonstrate that ketoamide inhibitors with proline ring analogs at the P2 position can function as broad-spectrum coronavirus M^{pro} inhibitors with potency approaching that of aldehyde inhibitors.

It was noted that the measured IC₅₀ values of ML1000 and GC-376 for SARSCoV2 M^{pro} were within experimental error of the theoretical limit of half of the enzyme concentration in the assay, hindering the ability to discern differences in potency between them. Thus, also measured were IC₅₀ values at a lower SARSCoV2 M^{pro} concentration of 20 nM (FIG. 24, panel C). In these conditions, ML1000 and GC-376 still demonstrated comparable IC₅₀ values of 12 and 14 nM, respectively, below the concentration of M^{pro} (i.e. the tight binding limit). Physiological temperature, compared to the 30° C. at which these assays are usually performed, could potentially increase structural flexibility and alter drug affinity of M^{pro}. However, no significant difference in potency was found between 37° C. and 30° C. for each inhibitor. These results indicate that ML1000 has high potency in vitro and represents the tightest-binding non-aldehyde SARSCoV2 M^{pro} inhibitor discovered so far.

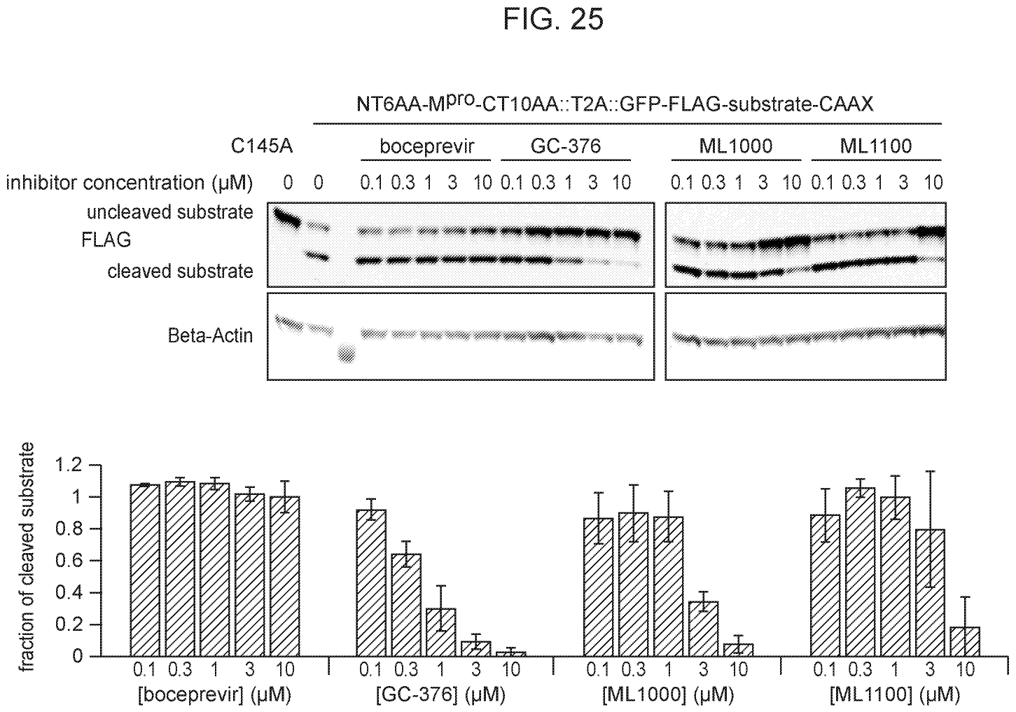

Next, the efficacy of the new inhibitors against SARSCoV2 M^{pro} was tested in human Huh7 cells by measuring the extent of cleavage of coexpressed substrate (FIG. 25). Here, observed was the rank order of effectiveness to be GC-376>ML1000>ML1100>boceprevir (FIG. 25). This assay does not directly measure the fraction of active M^{pro}. If a small fraction of active M^{pro} is sufficient to result in substrate cleavage, this would effectively increase the measured concentration needed to suppress protease activity. However, the assay does provide an assessment of the relative ability of a set of drugs to cross the cellular membrane and inhibit M^{pro} within living cells.

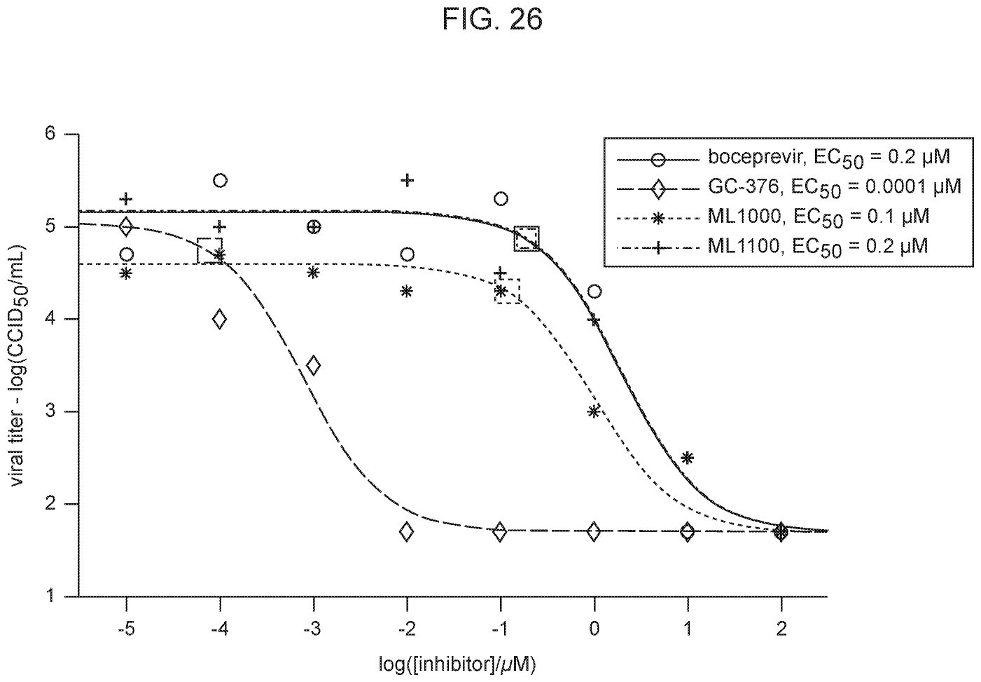

Finally, the ability of ML1000 and ML1100 to inhibit SARSCoV2 virus replication was tested in Caco-2 human intestinal cells. For comparison, also tested were boceprevir and GC-376. Boceprevir inhibited viral replication with an EC₅₀ of 0.2 μM, while GC-376 was even more potent, with a remarkably low EC₅₀ of 0.1 nM (table below, FIG. 26). ML1000 and ML1100 inhibited SARSCoV2 replication with EC₅₀ values of 0.1 and 0.2 μM, respectively (table below, FIG. 26). While the enhancement in anti-viral efficacy of ML1000 relative to boceprevir is not as large as the enhancement in enzyme inhibition observed in vitro, the EC₅₀ value of ML1000 is nevertheless the lowest reported to date for any non-aldehyde inhibitors of SARSCoV2 M^{pro}. In addition, ML1000 and ML1100 exhibited no cytotoxicity at the highest dose tested of 100 μM in Caco-2 cells and Huh-7 cells (table below), resulting in selectivity indices of >1000 and >500 respectively. These results confirm that ML1000 is a potent and selective ketoamide inhibitor of SARSCoV2 replication.

Antiviral Activity of ML1000 and ML1100 on SARSCoV2 Replication in Caco-2 Cells

| | EC₅₀ (μM) | CC₅₀ (μM) | Selectivity index |
|---|---|---|---|
| GC-376 | 0.0001 | >100 | >10⁶ |
| ML1000 | 0.1 | >100 | >1000 |
| ML1100 | 0.2 | >100 | >500 |
| Boceprevir | 0.2 | >100 | >500 |

EC₅₀: 50% effective antiviral concentration
CC₅₀: 50% cytotoxic concentration of compound without virus added
Selectivity index = CC₅₀/EC₅₀

### Methods for Example 4

### Inhibitors

ML1000 and ML1100 were synthesized under fee-for-service agreements by ACME Bioscience (Palo Alto, Calif., USA), and Chempartner (Shanghai, China), respectively. All other inhibitors were readily available: boceprevir (Cayman Chemical, ≥98%), narlaprevir (AdooQ, ≥98%), telaprevir (AdooQ Bioscience, ≥98%), GC-376 (AOBIOUS, ≥98%), ebselen (Cayman Chemical, ≥99%), disulfiram (LKT Laboratories, ≥98%), ritonavir (Santa Cruz Biotechnology, ≥98%).

Cell-Based M^{pro} Activity Assay in HEK293A and Huh7

Cell culture and transfection. HEK293A and HEK293FT cells were cultured at 37° C. in 5% CO₂ in Dulbecco's Modified Eagle's Medium (DMEM, Gibco) supplemented with 10% FBS and 100 U/mL penicillin and 100 μg/mL streptomycin. Huh7 cells were cultured at 37° C. in 5% CO₂ in Roswell Park Memorial Institute 1640 medium (RPMI 1640, Life Technologies) supplemented with 10% FBS and 100 U/mL penicillin and 100 μg/mL streptomycin.

M^{pro} activity assay in HEK293A. Cells were transfected with a pcDNA3.1/Puro-CAG plasmid containing the construct shown in FIG. 2 using Lipofectamine 3000 (Life Technologies) in Opti-MEM (Life Technologies) according to the manufacturer's protocol. Telaprevir and boceprevir were added 2 h post-transfection. 24 h post-transfection, cells were washed twice with PBS, then lysed with 50 μL hot LDS lysis buffer (50% 4×LDS Sample Buffer (NuPAGE, Life Technologies), 10% 2-mercaptoethanol), and DNA was sheared by sonication. After heating at 80-90° C. for 1-2 minutes, cell lysates were loaded onto 4%-12% or 12% Bis-Tris gels (NuPAGE, Life Technologies) along with a Precision Plus protein dual-color standard (Bio-Rad). Protein bands were transferred to PVDF membranes using a Trans-Blot Turbo Transfer System (Bio-Rad) and blocked with SuperBlock T20 (TBS) Blocking Buffer (Thermo Scientific). Membranes were probed with primary antibodies in SuperBlock T20 (TBS) Blocking Buffer and fluorophore-conjugated secondary antibodies in SuperBlock (TBS) Blocking Buffer (Thermo Scientific), with 3 washes in SuperBlock T20 (TBS) Blocking Buffer after each step. Membranes were imaged using an Odyssey imaging system (LI-COR). Western blots were quantified using ImageJ. The following primary antibodies were used for immunoblotting at the indicated dilutions: mouse monoclonal anti-FLAG (Sigma-Aldrich, F1804), 1:2000; rabbit polyclonal anti-beta Actin (Abcam, ab8227), 1:5000. Secondary antibodies were LI-COR 680RD goat-anti-rabbit and 800CW goat-anti-mouse, used at 1:5000 dilution each.

M^{pro} activity assay in Huh7. Lentiviruses were produced by transfecting HEK293FT cells with pLL3.7 plasmids containing the construct shown in FIG. 2 or a version with a catalytically dead M^{pro} C145A mutation and two helper plasmids (psPAX2, MD2G). Transfection was carried out with CalPhos™ Mammalian Transfection Kit (Takara Bio) at a ratio of pLL3.7:psPAX2:MD2G=20:15:6. The virus-containing supernatant was harvested 48 or 72 hours post transfection. Cell debris was removed with a 5 min 2000 g centrifugation, followed by a 0.45 μm filtration (Millipore). Virus titer was determined using Lenti-X™ GoStix™ Plus (Takara Bio), and Huh7 cells were infected at a MOI of 5. Drugs were added 24 hours post infection and cells were lysed and prepared for western blotting, as described above, 4 days post infection.

M^{pro} Expression and Purification

Modified versions of previous protocols based on HRV protease or SUMO protease processing of M^{pro} fusion proteins were used to obtain purified M^{pro} with either native or extended termini after expression in E. coli. Specifically, M^{pro} variants were cloned either as a GST-M^{pro}-His₆ or His₆-SUMO-M^{pro} fusion into a pET vector (Addgene plasmid #29666) using synthetic gene blocks for the M^{pro} portion of the SARS-CoV2 polyprotein (pp1ab residue 3264-3569) or the MHV1 polyprotein (pp1ab 3314-3624). In general, the His₆-SUMO-M^{pro} fusions produced higher yields of soluble protein compared to the GST-M^{pro}-His₆ constructs. This is likely due to toxicity and growth retardation associated with M^{pro} activation upon autocatalytic removal of the GST-tag during expression of the GST-M^{pro}-His₆ fusion. In contrast, the His₆-SUMO-M^{pro} fusion is produced in full-length and only becomes fully active after SUMO-tag removal during subsequent purification steps.

For SARSCoV2 M^{pro} variants, the plasmids were transformed into T7 Express lysY/I^{q} Competent E. coli (NEB). All cultures were grown in 2×YT media. Overnight cultures were used to inoculate larger cultures that were grown at 37° C. to OD₆₀₀˜0.8 before induction with 0.5 mM IPTG. After induction and 4-5 h growth at 24° C., the cells were harvested, and the pellets were frozen. Chemical lysis of the resuspended pellets was performed in BPER (Thermo Fisher) supplemented with 20 U/mL Pierce universal nuclease (ThermoFisher Scientific), and the supernatant cleared by 15 min of centrifugation at 15000 g. The soluble fraction was batch absorbed onto INDIGO-Ni resin (Cube Biotech) in a buffer with imidazole and NaCl added to 10 mM and 200 mM, respectively. The resin was loaded onto gravity flow columns and washed with 20 column volumes of wash buffer containing 50 mM Tris (pH 8), 25 mM Imidazole, and 300 mM NaCl. High purity protein was eluted in a buffer of 50 mM Tris (pH 8), 250 mM Imidazole, and 300 mM NaCl. Eluted fractions with high protein content were pooled and buffer exchanged into HRV protease cleavage buffer (50 mM Tris (pH 7.3), 150 mM NaCl, 1 mM DTT) or SUMO protease cleavage buffer (50 mM Tris (pH 8), 150 mM NaCl, 1 mM DTT). The proteins were cleaved by incubation with 1.5% (w/w) His-tagged HRV-3C protease (Millipore Sigma SAE0045) or 15 U/mg His-tagged SUMO protease (Millipore Sigma SAE0067) by overnight incubation at 4° C. The fully processed M^{pro} variants were then purified using reverse affinity chromatography to remove the His-tagged HRV and SUMO proteases and the cleaved His₆-tagged fusion-domains/peptides. Purity of the samples was checked on SDS-PAGE, and protein concentrations were determined based on A₂₈₀ and predicted extinction coefficients.

For MHV M^{pro}, expression from construct IV did not result in acceptable yields of M^{pro}-His₆. Furthermore, the expression of construct V and VI could be dramatically improved by adding 10 mM DTT to the lysis, wash, and elution buffers described above. All other steps and conditions were unchanged from the protocol described above for SARSCoV2 M^{pro}.

In-Vitro M^{pro} Inhibition Assays

The proteolytic activity of purified M^{pro} was primarily measured using a fluorogenic peptide, Covidyte IF670 (AAT Bioquest), that includes a far-red fluorophore iFluor™ 670 and a quencher Tide Quencher™ 5, TQ5. Upon cleavage of the iFluor-VNSTLQ/SGLRK(TQ5)M peptide by M^{pro}, energy transfer to the quencher decreases and the fluorescence intensity of iFluor increases. A well-plate reader (Tecan, Safire 2) was used to monitor the fluorescence with excitation at 640/20 nm and emission at 680/20 nm. All measurements were performed as bottom-reads from lid-covered black 96 well plates with clear bottoms. The in-vitro M^{pro} activity was measured in an assay buffer consisting of 50 mM Tris (pH 7.3), 50 mM NaCl, 1 mM EDTA, 2 mM DTT, and 1-2% DMSO. In specific experiment DTT was omitted from the buffer.

For the majority of IC₅₀ measurements, 100 nM M^{pro} was preincubated with varying concentrations of inhibitor for 30 min at 30° C. Next, the substrate was added to a final concentration of 2 μM and a final volume of 90 μL. The well-plate was immediately inserted into the plate reader, that had been preheated to 30° C., and the fluorescence intensity was monitored at 30 s intervals for 45 min. For selected experiments, the measurements were performed at 37° C. instead of 30° C.

The rate of substrate cleavage was extracted by linear fitting of the fluorescence signal increase as a function of time. The IC₅₀ curves were evaluated using the following equation in Prism (GraphPad):

$${v}_{r e l} = {v}_{min} + \frac{( {v}_{max} - {v}_{min} )}{1 + {( [ I ] / {IC}_{50} )}^{n}}$$

Here vᵣₑₗ is the experimentally measured rate of substrate cleavage normalized to the rate of substrate cleavage in the absence of inhibitor, I. The maximal and minimal rate of substrate cleavage in each experiment, vₘₐₓ and vₘᵢₙ, respectively, the Hill coefficient, n, and IC₅₀ are all fitting parameters in the non-linear fitting routine.

For GC-376 and ML1000, IC₅₀ values were also quantified at a lower M^{pro} concentration of 20 nM in attempt to escape the tight binding regime. For these experiments, a substrate with faster reaction rate, Covidyte TF670 (AAT Bioquest), was used to counteract the reduced M^{pro} activity and increase the sensitivity of the assay. This substrate consists of a far-red fluorophore Tide Fluor™ 5, TF5, and the TQ5 quencher linked by a peptide substrate, TF5-KTSAVLQ/SGFRKME(TQ5)M (SEQ ID NO:4).

Cytotoxicity Assay in Huh7 Cells

24 h prior to the drug treatment, 10000 Huh7 cells were seeded in 100 μl culture medium (DMEM with 10% FBS, 2 mM L-glutamine, penicillin-streptomycin) in a 96-well white microplate (Greiner Bio-One, Austria) pre-coated with poly-D-lysine (10 μg/ml, Sigma). The next day, the culture medium was replaced with assay medium (DMEM with 5% FBS, 2 mM L-glutamine, penicillin-streptomycin, 1% DMSO) containing inhibitors at the desired concentration (1-100 μM). Staurosporine, a non-selective protein kinase inhibitor known to induce apoptosis, was used as a positive control (0.01 μM-1 μM). After 48 h, cell viability was determined using the CellTiter-Glo 2.0 kit (Promega, USA) according to the instructions of the manufacturer. The bioluminescence signal was measured on a multi-mode microplate reader (Tecan, Safire 2).

Antiviral and Cytotoxicity Assays in Caco-2 Cells

The tested M^{pro} inhibitors were serially diluted from 10 mM DMSO stocks using eight log₁₀ dilutions in test medium (MEM supplemented with 2% FBS and 50 μg/mL gentamicin) yielding a concentration range of 10 μM-100 μM. Each dilution was added to 5 wells of a 96-well plate with 80-100% confluent Caco-2 cells. Three wells of each dilution were infected with virus, and two wells remained uninfected as toxicity controls. Six wells were infected and untreated as virus controls, and six wells were uninfected and untreated as cell controls. SARSCoV2 (USA_WA1/2020 strain passaged twice in Vero 76 cells in MEM supplemented with 2% fetal bovine serum and 50 μg/ml gentamicin to prepare a working stock) was prepared at a multiplicity of infection (MOI) that would yield measurable virus titers within 72 hours. Plates were incubated at 37±2° C. and 5% CO₂.

For virus yield reduction assays, the supernatant fluid from each condition was collected on day 3 post-infection (3 wells pooled) and tested for virus titer using an endpoint dilution in Vero 76 cells. The virus titer was determined by visual observation of cells under a light microscope on day 5 post-infection. Viral titers, VT, were quantified on a logarithm scale in the form of CCID₅₀/mL using the Reed Muench equation. The data was fitted to the following equation:

$$V T = V {T}_{min} + \frac{V {T}_{max} - V {T}_{min}}{1 + ( [ I ] / {VT}_{mid} )}$$

The 50% effective concentration, EC₅₀, was defined as the concentration of inhibitor were VT reaches VTₘₐₓ-log 2 and extracted from the fitted curves.

Drug cytotoxicity was also assayed on day 3 in a neutral red viability assay. Plates were stained with dye for 2 hours (±15 minutes). Supernatant dye was removed, wells rinsed with PBS, and the incorporated dye extracted in 50:50 Sorensen citrate buffer/ethanol for >30 minutes before measuring the optical density at 540 nm, OD₅₄₀. OD₅₄₀ was used to calculate the relative viability to non-exposed cell controls.

### Example 5—A Cell-Based Bioluminescence Assay for Characterization of Protease Inhibitors

Described herein is the development of a cell-based bioluminescence assay for characterization of protease inhibitors—in this particular example, SARS-CoV-2 Mpro inhibitors. The assay is based on a fusion reporter protein, which contains a native SARS-CoV-2 Mpro, with the autocatalytic cleavage sites at both ends, fused within a split NanoLuc luciferase, NanoBiT. In this example, SARS-CoV-2 Mpro was inserted in between the SmBiT and LgBiT of the split luciferase, NanoBiT, using flexible linkers that includes the natural cleavage sites of SARS-CoV-2 Mpro. Provided in the table below is the amino acid sequence of the reporter protein employed in this example, as well as a cDNA sequence that encodes the reporter protein. For the amino acid sequence, alternating underlining indicates the nine domains shown in the schematic illustration of the reporter protein in FIG. 19 (top).

| | |
|---|---|
| Amino Acid | MVTGYRLFEEILAGGGGSGMTSAVLQSGFRKMAFPSGK |
| Sequence | VEGCMVQVTCGTTTLNGLWLDDVVYCPRHVICTSEDML |
| (SEQ ID | NPNYEDLLIRKSNHNFLVQAGNVQLRVIGHSMQNCVLK |
| NO: 5) | LKVDTANPKTPKYKFVRIQPGQTFSVLACYNGSPSGVY |
| | QCAMRPNFTIKGSFLNGSCGSVGFNIDYDCVSFCYMHH |
| | MELPTGVHAGTDLEGNFYGPFVDRQTAQAAGTDTTITV |
| | NVLAWLYAAVINGDRWFLNRFTTTLNDFNLVAMKYNYE |
| | PLTQDHVDILGPLSAQTGIAVLDMCASLKELLQNGMNG |
| | RTILGSALLEDEFTPFDVVRQCSGVTFQSAVKRTIKGT |
| | TSGGGSGGVFTLEDFVGDWEQTAAYNLDQVLEQGGVSS |
| | LLQNLAVSVTPIQRIVRSGENALKIDIHVIIPYEGLSA |
| | DQMAQIEEVFKVVYPVDDHHFKVILPYGTLVIDGVTPN |
| | MLNYFGRPYEGIAVFDGKKITVTGTLWNGNKIIDERLI |
| | TPDGSMLFRVTINSGGSGGSYPYDVPDYA |
| cDNA | ATGGTGACCGGCTACCGGCTGTTCGAGGAGATTCTGGC |
| Sequence | GGGTGGCGGAGGATCTGGAATGACCTCAGCTGTTTTGC |
| (SEQ ID | AGAGTGGTTTTAGAAAAATGGCATTCCCATCTGGTAAA |
| NO: 6) | GTTGAGGGTTGTATGGTACAAGTAACTTGTGGTACAAC |
| | TACACTTAACGGTCTTTGGCTTGATGACGTAGTTTACT |
| | GTCCAAGACATGTGATCTGCACCTCTGAAGACATGCTT |
| | AACCCTAATTATGAAGATTTACTCATTCGTAAGTCTAA |
| | TCATAATTTCTTGGTACAGGCTGGTAATGTTCAACTCA |
| | GGGTTATTGGACATTCTATGCAAAATTGTGTACTTAAG |
| | CTTAAGGTTGATACAGCCAATCCTAAGACACCTAAGTA |
| | TAAGTTTGTTCGCATTCAACCAGGACAGACTTTTTCAG |
| | TGTTAGCTTGTTACAATGGTTCACCATCTGGTGTTTAC |
| | CAATGTGCTATGAGGCCCAATTTCACTATTAAGGGTTC |
| | ATTCCTTAATGGTTCATGTGGTAGTGTTGTTTTAACAT |
| | AGATTATGACTGTGTCTCTTTTTGTTACATGCACCATA |
| | TGGAATTACCAACTGGAGTTCATGCTGGCACAGACTTA |
| | GAAGGTAACTTTTATGGACCTTTTGTTGACAGGCAAAC |
| | AGCACAAGCAGCTGGTACGGACACAACTATTACAGTTA |
| | ATGTTTTAGCTTGGTTGTACGCTGCTGTTATAAATGGA |
| | GACAGGTGGTTTCTCAATCGATTTACCACAACTCTTAA |
| | TGACTTTAACCTTGTGGCTATGAAGTACAATTATGAAC |
| | CTCTAACACAAGACCATGTTGACATACTAGGACCTCTT |
| | TCTGCTCAAACTGGAATTGCCGTTTTAGATATGTGTGC |
| | TTCATTAAAAGAATTACTGCAAAATGGTATGAATGGAC |
| | GTACCATATTGGGTAGTGCTTTATTAGAAGATGAATTT |
| | ACACCTTTTGATGTTGTTAGACAATGCTCAGGTGTTAC |
| | TTTCCAAAGTGCAGTGAAAAGAACAATCAAGGGTACAA |
| | CTAGTGGCGGAGGCTCTGGAGGAGTCTTCACACTCGAA |
| | GATTTCGTTGGGGACTGGGAACAGACAGCCGCCTACAA |
| | CCTGGACCAAGTCCTTGAACAGGGAGGTGTGTCCAGTT |
| | TGCTGCAGAATCTCGCCGTGTCCGTAACTCCGATCCAA |
| | AGGATTGTCCGGAGCGGTGAAAATGCCCTGAAGATCGA |
| | CATCCATGTCATCATCCCGTATGAAGGTCTGAGCGCCG |
| | ACCAAATGGCCCAGATCGAAGAGGTGTTTAAGGTGGTG |
| | TACCCTGTGGATGATCATCACTTTAAGGTGATCCTGCC |
| | CTATGGCACACTGGTAATCGACGGGGTTACGCCGAACA |
| | TGCTGAACTATTTCGGACGGCCGTATGAAGGCATCGCC |
| | GTGTTCGACGGCAAAAAGATCACTGTAACAGGGACCCT |
| | GTGGAACGGCAACAAAATTATCGACGAGCGCCTGATCA |
| | CCCCCGACGGCTCCATGCTGTTCCGAGTAACCATCAAC |
| | AGCGGAGGTAGTGGTGGTAGCTACCCTTACGATGTCCC |
| | TGACTACGCG |

After the reporter vector is transiently expressed in mammalian cells, the autocatalytic activity of Mpro leads to the separation of the two split NanoLuc luciferase components SmBiT and LgBiT, resulting in low background bioluminescence. In the presence of protease inhibitors, the fusion protein remains intact, retaining its ability to reconstitute the NanoBiT luciferase activity. Thus, in this assay, the reconstituted NanoBiT luciferase acts as a turn-on biosensor of SARS-CoV-2 Mpro inhibition, making it ideal for platereader-based high-throughput screening. A feature of the turn-on reporter is a reduced risk of false positives caused by compound induced cell-toxicity. The fusion reporter protein and assay are schematically illustrated in FIG. 19.

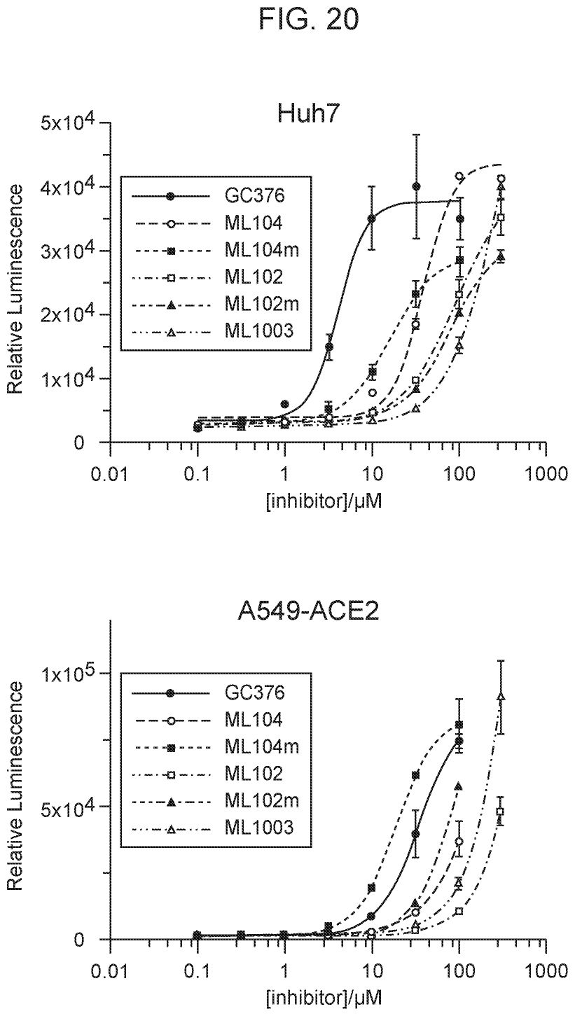

Shown in FIG. 20 are results produced by the assay in Huh7 and A549-ACE2 cells using selected compounds. Huh7 or A549-ACE2 cells were transfected using lipofectamine in white 96 well plates. After 2 h, compounds were added at a range of concentrations to individual wells across the well plate. Following 48 h of incubation with expression of the reporter in the presence of compound, the cells were lyzed and the bioluminescent signal developed using Nano-Glo luciferase assay (Promega) or NLuc GFLOW assay (NanoLight technology). However, the assay is not limited to usage with cell lysates, as other kits allow development of bioluminescence signals from live cells. The increase in bioluminescence as a function of compound concentration was plotted and fitted to a logistic function as shown in FIG. 20. In general there is good correlation between this assay and the benchmark of viral replication presented in Example 9. However, in contrast to the experiments discussed in Example 9, the assay presented here can be conveniently performed under biosafety level 2 conditions that are more accessible.

### Example 6—Inhibition of Mpro of Different Coronaviruses

Inhibition of Mpro of different coronaviruses by a group of designed compounds was tested. Shown in the table below are IC50 values for the inhibition of recombinantly expressed and purified Mpro of different coronaviruses. In addition to the Mpro variants described in Example 4, affinities of the compounds towards mature Mpro of MERS (pp1ab 3248-3553) and HCoV229E (pp1ab 2966-3267) were also tested.

| | IC50 [μM] | | | | | |
|---|---|---|---|---|---|---|
| | SARSCov2 Mpro | MERS Mpro | MHV Mpro | HCoV229E Mpro | HCathepsin B | HCathepsin L |
| Boceprevir | 4.1 | — | 15 | — | 4.5 | 0.4 |
| Telaprevir | >20 | — | — | — | 36 | 25 |
| GC376 | 0.02 | 0.09 | 0.05 | 0.19 | 0.001 | 0.001 |
| ML1000 | 0.01 | 0.53 | 0.13 | 0.13 | 5.9 | 3.2 |
| ML1100 | 0.07 | 1.3 | 0.31 | 1.2 | 62 | 38 |
| ML1001 | 0.02 | 0.26 | 0.07 | 0.50 | 42 | 33 |
| ML1002 | 0.02 | 0.19 | — | 0.26 | — | — |
| ML1002m | 0.02 | 0.25 | — | 0.40 | — | — |
| ML1003 | 0.04 | 0.16 | — | 0.35 | — | — |
| ML101 | 0.09 | 1.4 | 0.71 | 0.61 | 2.7 | 1.3 |
| ML102 | 0.09 | 1.2 | 0.98 | 0.29 | 2.2 | 0.8 |
| ML102m | 0.12 | 1.1 | — | 0.72 | — | — |
| ML104 | 0.03 | 0.19 | 0.25 | 0.44 | 17 | 11 |
| ML104m | 0.03 | 0.29 | — | 0.23 | — | — |
| ML105 | 0.02 | 0.34 | 0.06 | 0.01 | 0.7 | 0.3 |

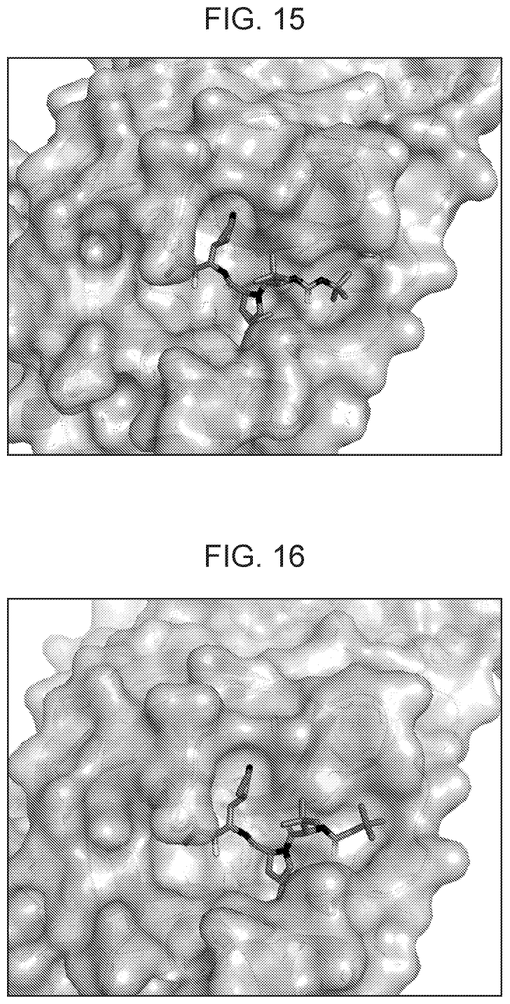

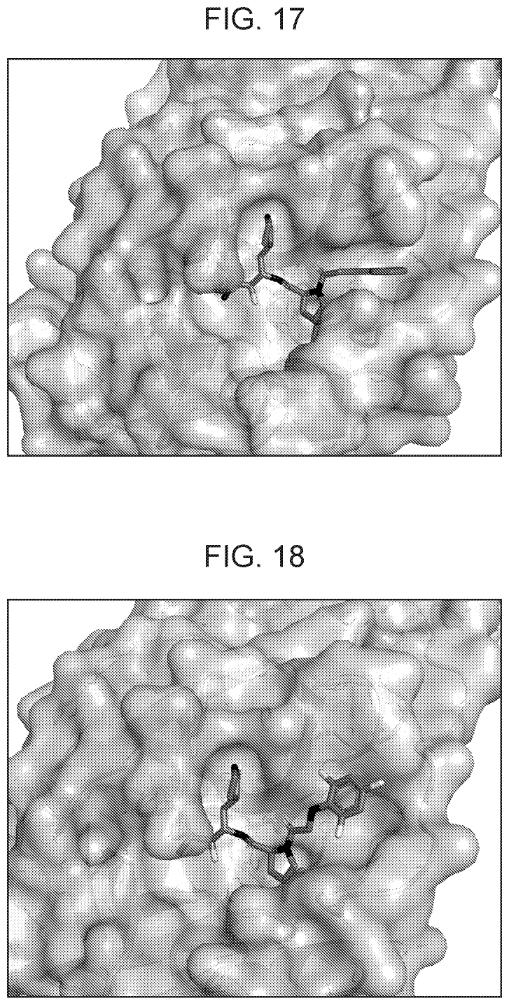

The data shows that the designed compounds all inhibit SARS-CoV-2 Mpro with high affinity in the nM range. In particular, the following compounds show IC50s against SARS-CoV-2 Mpro below 50 nM: ML1000, ML1001, ML1002, ML1003, ML104, ML104m, ML105. Thus, these designs, with a P1 of gamma-lactamyl, P2 of boceprevir proline derivative, and the information provided herein by the exploration of P3 and P4 provide the basis for improved Mpro inhibitors. Together with the structural data of FIGS. 15-18 this IC50 dataset provides a basis for further lowering IC50 values and/or improving other therapeutically important factors through rational approaches. For example, it allows those skilled in the art to identify neutral permutations of the compounds that can improve the pharmacological profile of the compounds without negatively impacting the high affinity towards Mpro established herein. In particular, it is noted that the cocrystal structure of ML104 (FIG. 18) shows a conformation that is significantly different from the other compounds explored herein, while simultaneously maintaining an IC50 of 30 nM. From this example, but with no limitation to the specific structure, it naturally follows that other trihalophenylaminomethyl, substituted phenylaminomethyl, trihalophenoxymethyl, substituted phenoxymethyl could replace the 2,4,6-trifluorophenylaminomethyl of ML104 and ML104m and potentially improve the therapeutic utility of this scaffold.

In addition the compounds of the ML-series show broad inhibitory effects towards Mpro of other coronavirus variants. Thus, the compounds of the present disclosure have the potential to serve as efficient inhibitors of Mpro proteins from diverse coronaviruses.

As a test for protease specificity, the compounds were also tested against human Cathepsins B and L. The results are shown in the table below. In contrast to the aldehyde-based GC-376, that shows strong cross reactivity with the cathepsins, the IC50 values of most of the tested ketoamide-based compounds of the ML-series are in the μM range. Thus, the explored designs show the potential to specifically target exogenous viral protease over endogenous host proteases. This should minimize the risk for adverse side-effects of the compounds. These properties are not limited to the ketoamide warheads presented here, but identifies the possibility to obtain high specificity by choice of the electrophile incorporated into the compounds. The present work provides the basis for selecting other electrophilic warheads of interest.

Methods for Example 6

Expression and purification of Mpro variants was performed using the procedure described in the Methods section of Example 4. The assay was performed as described in the Methods section of Example 4. A fluorogenic reporter (Covidyte TF670 or IF670, AAT Bioquest) was added and used to monitor the protease activity after 30 min of incubation of the Mpro variants in the presence of varying concentrations of compound. In these assays the final concentration of the Mpro variants were: 20 nM SARSCoV2 Mpro, 100 or 200 nM MERS Mpro, 100 nM MHV Mpro, and 20 nM HCoV229E 20 nM, respectively.

Human Cathepsin B and L were obtained from R&D Systems. The assays were performed with 0.05 ng/uL of Cathepsin B or 0.01 ng/uL Cathepsin L in 50 mM MES buffer pH 5.5 with 2 mM DTT at room temperature. First, the cathepsins were preactivated by incubation in assay buffer for 30 min prior to 30 min incubation with the compounds. After incubation with the compounds, 20 μM Z-LR-AMC fluorogenic peptide substrate (R&D Systems) was added to monitor the proteolytic activity. The relative activity of the protease was obtained by normalization against a control that contained no inhibitor.

IC50 values were in all cases extracted as described in the Methods section of Example 4.

### Example 7—Melting Temperature Measurements

The change in protein melting temperature of 3 μM SARS-CoV-2 mature Mpro or the corresponding inactive Mpro C145A mutant in the presence of 30 μM of selected compounds was assessed by differential scanning fluorometry using a BioRad CFX96 instrument. The samples were heated from 25-100° C. at ˜1° C./min and fluorescence of SYPRO Orange (5× final concentration, ThermoFisher Scientific) was monitored in the FRET channel. Results are shown in the table below.

| ΔTm [° C.] | Mpro | Mpro C145A |
|---|---|---|
| Boceprevir | 3.6 | 0 |
| Telaprevir | 0.9 | −0.1 |
| GC376 | 17.9 | 0 |
| ML1000 | 20.2 | 0.2 |
| ML1100 | 20.7 | 0 |
| ML1001 | 21.6 | 1.2 |
| ML1002 | 21.6 | 0.1 |
| ML1002m | 21 | 0.1 |
| ML1003 | 21.3 | 0.1 |
| ML101 | 15.5 | 0 |
| ML102 | 15.5 | 0 |
| ML102m | 15.2 | 0 |
| ML104 | 17.5 | 0 |
| ML104m | 17.1 | 0 |
| ML105 | 18.6 | 0 |

In contrast to boceprevir and telaprevir, the designed compound stabilized the protein to a larger degree, indicating that the covalently bound compounds of the ML series better complement the binding pocket of SARS-CoV-2 Mpro. In the absence of covalent bond formation to SARS-CoV-2 Mpro C145A, only ML1001 showed a substantial stabilization of the protein scaffold. Thus, the P3 and P4 groups of this particular design may favor increased residence time of ML1001 in the protease binding site before covalent bond formation and after reversible cleavage of the thiohemiketal. This observation provides the basis for modifications of P3 and P4 groups by considering properties of alternative substituents such as, but not limited to, flexibility, bulk, hybridization, and electrostatics. Examples of this include, but are not limited to, the incorporation of fluorinated alkyls at P4.

### Example 8—Cytotoxicity Measurements

The cytotoxicity of selected compounds was assessed in Caco2, Huh7 and A549-ACE2 cells after a 72h incubation with the compounds. Specifically, cell viability in the presence of compound was quantified using the CellTiter-Glo 2.0 assay (Promega) and normalized against controls containing an equivalent amounts of DMSO. Results are shown in the table below.

| CC50 [μM] | Caco-2 | Huh7 | A549-ACE2 |
|---|---|---|---|
| Boceprevir | >100 | >100 | >100 |
| Telaprevir | — | 10-100 | 10-100 |
| GC376 | >100 | >100 | >100 |
| ML1000 | >100 | >100 | >100 |
| ML1100 | >100 | >100 | >100 |
| ML1001 | >100 | >100 | >100 |
| ML1002 | — | >100 | >100 |
| ML1002m | — | >100 | >100 |
| ML1003 | — | >100 | >100 |
| ML101 | — | >100 | >100 |
| ML102 | — | >100 | >100 |
| ML102m | — | >100 | >100 |
| ML104 | >100 | >100 | >100 |
| ML104m | — | >100 | >100 |
| ML105 | >100 | >100 | >100 |

As shown, no significant cytotoxicity was observed in any of the cell lines at 100 μM of the designed compounds. This illustrates that the molecular scaffold of the present disclosure provides compounds with good safety profiles.

### Example 9—Inhibition of Viral Replication

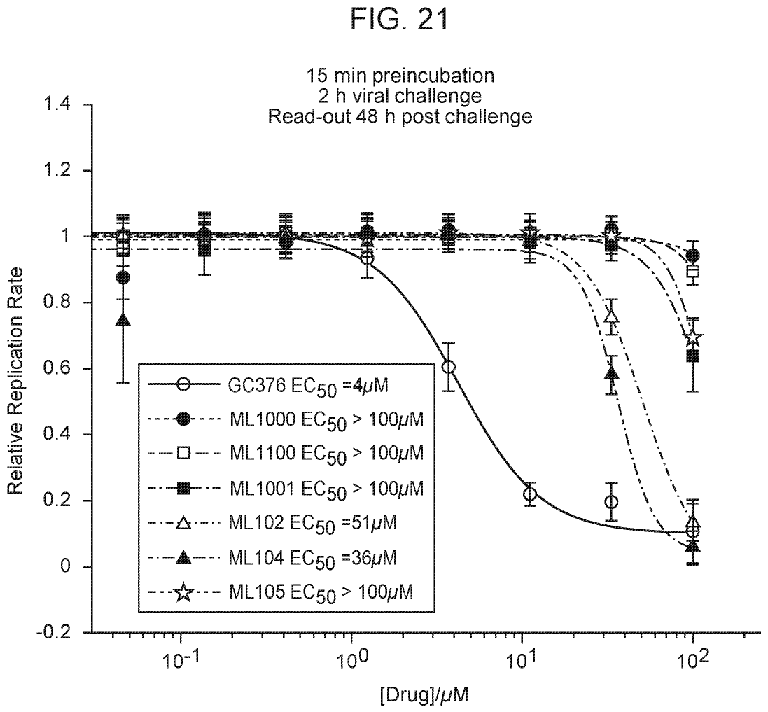

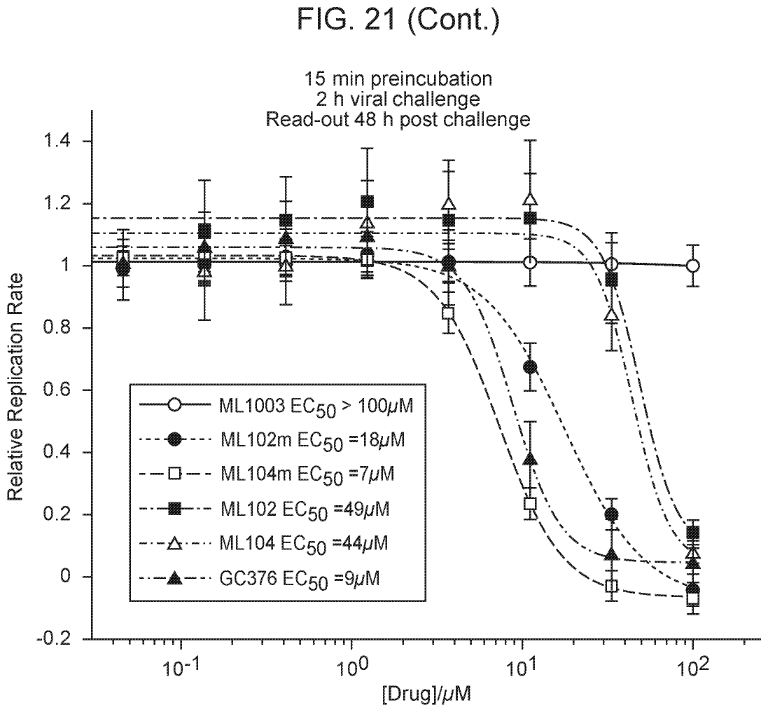

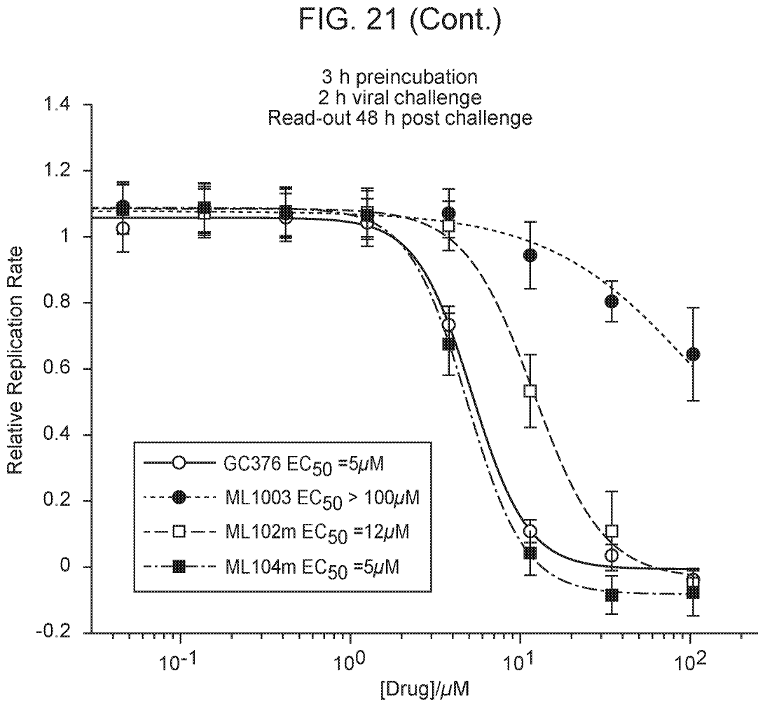

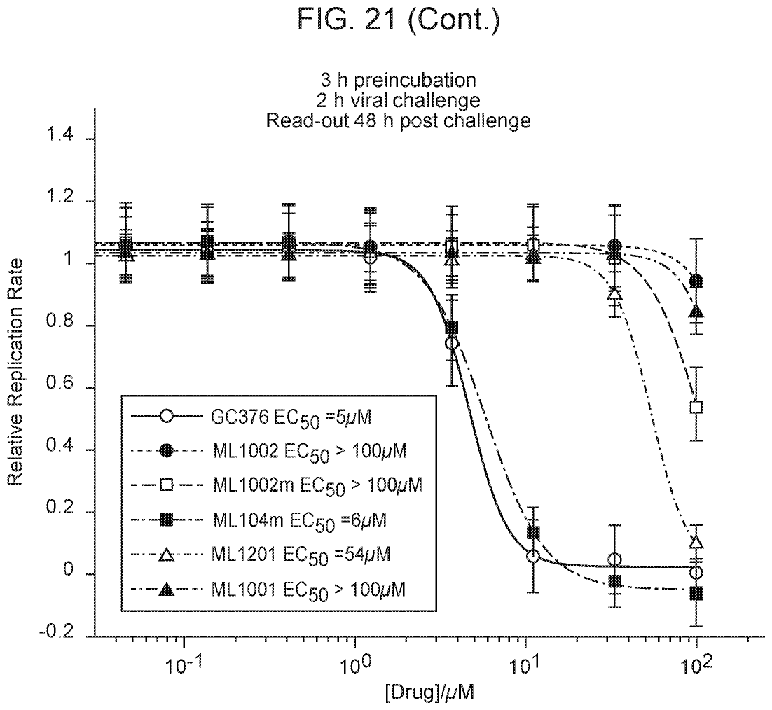

Inhibition of viral replication in A549-ACE2 cells was tested for selected compounds. Results are shown in FIG. 21. This dataset shows that compounds of the present disclosure are capable of inhibiting SARS-CoV-2 replication in a coronavirus disease-relevant epithelial cell line derived from human lung tissue. Comparison of ML102 to ML102m and ML104 to ML104m shows that methylation of the N-atom of the ketoamide group can be utilized to improve the cell permeability of the compounds. This is supported by the data in Example 6 demonstrating that this improvement is not due to increased affinity towards Mpro. From the N-methyl substitutions incorporated into ML102m and ML104m, it follows that other substitutions that provide secondary and tertiary ketoamides may improve the cell permeability of the compounds. Other alternative warheads, for example a nitrile, provide similar benefits by removing non-essential hydrogen-bond donors. Additional ways to improve permeability while maintaining affinity towards Mpro involves exchanging P1 to other groups that in contrast to the gamma-lactamyl contain only a hydrogen-bond acceptor and no hydrogen-bond donor, non-limiting examples of which are pyridinyls.

Methods for Example 9

SARS-CoV-2-nLuc (doi.org/10.1038/s41586-020-2708-8) in the form of a passage 1 stock was a kind gift from Jacob Hou and Ralph Baric. The virus was passaged twice in VeroE6 cells and titered by plaque assay on VeroE6 cells. Drugs were added in DMEM with 2% FBS to A549-ACE2 cells (doi.org/10.1038/s41467-020-19619-7), which were a gift from Ralf Bartenschlager, in 96-well plates. Control wells were treated with equal concentrations of DMSO. Next, cells were then infected at MOI 0.1 in the presence of drug, washed, and incubated with drug for 48 hrs before assessment by lytic Nano-Glo assay (Promega) and read on a GloMax plate reader (Promega). Infections and plate reading occurred inside class II biosafety cabinets under biosafety level 3 conditions. The logarithm of the luminescent signal was used as a measure of the viral replication rate. The relative reduction of the replication rate as a function of the drug concentration was fitted to a logistic function to extract the EC50 values as the concentration resulting in a 50% reduction in the replication rate.

### Example 10—Absorption, Distribution, Metabolism, and Excretion (ADME)

Selected compounds were tested for absorption, distribution, metabolism, and excretion (ADME). Results are shown in the table below.

| | Kinetic | Caco-2 Permeability | | Plasma Stability | |
|---|---|---|---|---|---|
| | Solubility | Papp A-B | Papp B- | | Mouse |
| | in PBS | [10-6 | A[10-6 | Human | T1/2 |
| ADME | [μM] | cm/s] | cm/s] | T1/2 [min] | [min] |
| Boceprevir | 73 | 2 | 29 | 495 | 28 |
| GC376 | 71 | <0.3 | 5 | 95 | 232 |
| ML1000 | 87 | <0.2 | 0.4 | 109 | 23 |
| ML1001 | 66 | <0.2 | 0.5 | 330 | 26 |
| ML1002 | >100 | <0.2 | <0.2 | — | 21 |
| ML1002m | >100 | 0.4 | 0.7 | — | 170 |
| ML101 | 79 | <0.2 | 1.3 | ∞ | 8 |
| ML102 | 83 | <0.2 | 0.7 | ∞ | 18 |
| ML102m | >100 | 0.4 | 1.8 | — | 45 |
| ML104 | 86 | <0.2 | 0.9 | ∞ | 100 |
| ML104m | 98 | 0.5 | 2.5 | — | 81 |
| ML105 | 80 | <0.2 | 0.3 | ∞ | 20 |

The tested compounds of the ML-series exhibit good solubility in physiological buffer and overall good stability in plasma from humans and mice. Upon comparison of the following sets of compounds, ML1002 versus ML1002m, ML102 versus ML102m, and ML104 versus ML104m, the data supports the previous observation that methylation of the ketoamide improves cell permeability.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A compound having the formula: 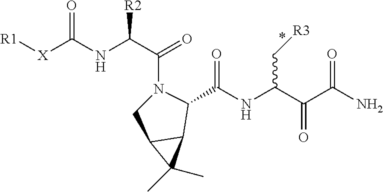
wherein:
R1 is 1,1-dimethylethyl, 1-methyl-1-fluoroethyl, 1-methylethyl, 1,1-difluoroethyl, 1-fluoroethyl, ethyl, trifluoromethyl, difluoromethyl, fluoromethyl, methyl, 2-fluoroethyl, 2,2-difluoroethyl, 2,2,2-trifluoroethyl, 1-methyl-2-fluoroethyl, 1-methyl-2,2-difluoroethyl, 1-methyl-2,2,2-trifluoroethyl, alkyl, substituted alkyl, aryl, substituted aryl, heterocycle, or substituted heterocycle;
R2 is 1,1-dimethylethyl, 1-methyl-1-fluoroethyl, 1-methylethyl, 1,1-difluoroethyl, 1-fluoroethyl, trifluoromethyl, difluoromethyl, 1-methyl-2-fluoroethyl, hydrogen, halogen, alkyl, substituted alkyl, aryl, substituted aryl, heterocycle, or substituted heterocycle;
R3 is gamma-lactamyl, cyclopentanonyl, cyclopentyl, beta-lactamyl, cyclobutanonyl, cyclobutyl, acetonyl, acetyl, gamma-lactonyl, furanonyl, pyrrolonyl, cyclopentenonyl, oxazolonyl, imidazolonyl, alkyl, substituted alkyl, aryl, substituted aryl, heterocycle, or substituted heterocycle;
X is CH₂, NH, or O; and
the bond indicated by the asterisk is a single or double bond.

2. The compound of claim 1, wherein the compound is:

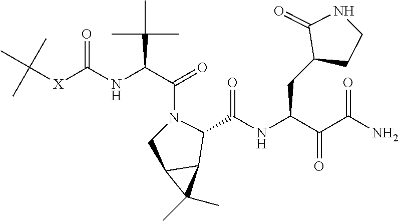

3. The compound of claim 2, wherein the compound is:

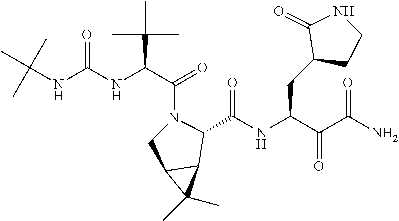 [SMILES: CC(C)(C)NC(=O)N[C@H](C(=O)N1C[C@H]2[C@@H]([C@H]1C(=O)N[C@@H](C[C@@H]1CCNC1=O)C(=O)C(N)=O)C2(C)C)C(C)(C)C]

4. A compound having the formula: 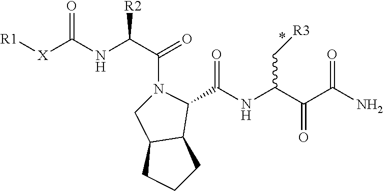
wherein:
R1 is 1,1-dimethylethyl, 1-methyl-1-fluoroethyl, 1-methylethyl, 1,1-difluoroethyl, 1-fluoroethyl, ethyl, trifluoromethyl, difluoromethyl, fluoromethyl, methyl, 2-fluoroethyl, 2,2-difluoroethyl, 2,2,2-trifluoroethyl, 1-methyl-2-fluoroethyl, 1-methyl-2,2-difluoroethyl, 1-methyl-2,2,2-trifluoroethyl, alkyl, substituted alkyl, aryl, substituted aryl, heterocycle, or substituted heterocycle;
R2 is 1,1-dimethylethyl, 1-methyl-1-fluoroethyl, 1-methylethyl, 1,1-difluoroethyl, 1-fluoroethyl, trifluoromethyl, difluoromethyl, 1-methyl-2-fluoroethyl, hydrogen, halogen, alkyl, substituted alkyl, aryl, substituted aryl, heterocycle, or substituted heterocycle;
R3 is gamma-lactamyl, cyclopentanonyl, cyclopentyl, beta-lactamyl, cyclobutanonyl, cyclobutyl, acetonyl, acetyl, gamma-lactonyl, furanonyl, pyrrolonyl, cyclopentenonyl, oxazolonyl, imidazolonyl, alkyl, substituted alkyl, aryl, substituted aryl, heterocycle, or substituted heterocycle;
X is CH₂, NH, or O; and
the bond indicated by the asterisk is a single or double bond.

5. The compound of claim 4, wherein the compound is:

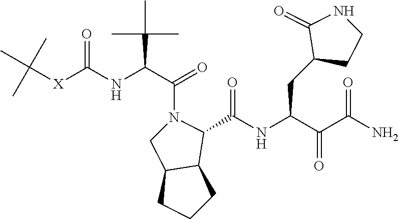

6. The compound of claim 5, wherein the compound is:

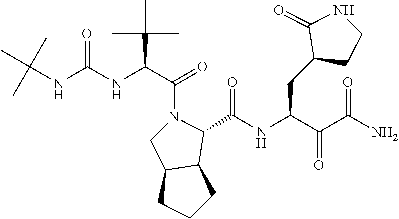 [SMILES: CC(C)(C)NC(=O)N[C@H](C(=O)N1C[C@@H]2CCC[C@@H]2[C@H]1C(=O)N[C@@H](C[C@@H]1CCNC1=O)C(=O)C(N)=O)C(C)(C)C]

7. A compound having the formula: 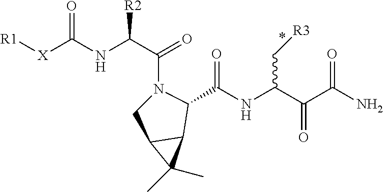
wherein:
R1 is 1,1-dimethylethyl, 1-methyl-1-fluoroethyl, 1-methylethyl, 1,1-difluoroethyl, 1-fluoroethyl, ethyl, trifluoromethyl, difluoromethyl, fluoromethyl, methyl, 2-fluoroethyl, 2,2-difluoroethyl, 2,2,2-trifluoroethyl, 1-methyl-2-fluoroethyl, 1-methyl-2,2-difluoroethyl, 1-methyl-2,2,2-trifluoroethyl, alkyl, substituted alkyl, aryl, substituted aryl, heterocycle, or substituted heterocycle;
R2 is 1,1-dimethylethyl, 1-methyl-1-fluoroethyl, 1-methylethyl, 1,1-difluoroethyl, 1-fluoroethyl, trifluoromethyl, difluoromethyl, 1-methyl-2-fluoroethyl, hydrogen, halogen, alkyl, substituted alkyl, aryl, substituted aryl, heterocycle, or substituted heterocycle;
R3 is gamma-lactamyl, cyclopentanonyl, cyclopentyl, beta-lactamyl, cyclobutanonyl, cyclobutyl, acetonyl, acetyl, gamma-lactonyl, furanonyl, pyrrolonyl, cyclopentenonyl, oxazolonyl, imidazolonyl, alkyl, substituted alkyl, aryl, substituted aryl, heterocycle, or substituted heterocycle;
R4 is hydrogen, halomethyl, hydroxymethyl, diazomethyl, acyloxymethyl, ketone, ketoamide, ketoacid, ketoester, alkyl, substituted alkyl aryl, substituted aryl, heterocycle, or substituted heterocycle;
X is CH₂, NH, or O; and
the bond indicated by the asterisk is a single or double bond.

8. A compound having the formula: 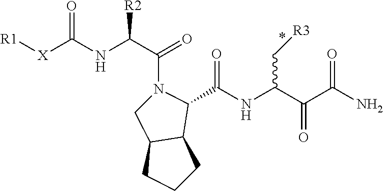
wherein:
R1 is 1,1-dimethylethyl, 1-methyl-1-fluoroethyl, 1-methylethyl, 1,1-difluoroethyl, 1-fluoroethyl, ethyl, trifluoromethyl, difluoromethyl, fluoromethyl, methyl, 2-fluoroethyl, 2,2-difluoroethyl, 2,2,2-trifluoroethyl, 1-methyl-2-fluoroethyl, 1-methyl-2,2-difluoroethyl, 1-methyl-2,2,2-trifluoroethyl, alkyl, substituted alkyl, aryl, substituted aryl, heterocycle, or substituted heterocycle;
R2 is 1,1-dimethylethyl, 1-methyl-1-fluoroethyl, 1-methylethyl, 1,1-difluoroethyl, 1-fluoroethyl, trifluoromethyl, difluoromethyl, 1-methyl-2-fluoroethyl, hydrogen, halogen, alkyl, substituted alkyl, aryl, substituted aryl, heterocycle, or substituted heterocycle;
R3 is gamma-lactamyl, cyclopentanonyl, cyclopentyl, beta-lactamyl, cyclobutanonyl, cyclobutyl, acetonyl, acetyl, gamma-lactonyl, furanonyl, pyrrolonyl, cyclopentenonyl, oxazolonyl, imidazolonyl, alkyl, substituted alkyl, aryl, substituted aryl, heterocycle, or substituted heterocycle;
R4 is hydrogen, halomethyl, hydroxymethyl, diazomethyl, acyloxymethyl, ketone, ketoamide, ketoacid, ketoester, alkyl, substituted alkyl aryl, substituted aryl, heterocycle, or substituted heterocycle;
X is CH₂, NH, or O; and
the bond indicated by the asterisk is a single or double bond.

9. A compound having the formula: 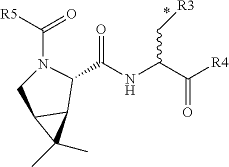
wherein:
R5 is pyrazole, substituted pyrazole, imidazole, substituted imidazole, piperidine, substituted piperidine, pyridine, substituted pyridine, pyridone, substituted pyridone, indole, methoxyindole, other substituted indole, isoindole, substituted isoindole, purine, substituted purine, benzyloxy, substituted benzyloxy, benzylamino, substituted benzylamino, phenoxymethyl, substituted phenoxymethyl, phenylaminomethyl, substituted phenylaminomethyl, phenylethyl, substituted phenylethyl, alkyl, substituted alkyl, aryl, substituted aryl, heterocycle, or substituted heterocycle;
R3 is gamma-lactamyl, cyclopentanone, cyclopentyl, beta-lactamyl, cyclobutanonyl, cyclobutyl, acetonyl, acetyl, gamma-lactonyl, furanonyl, pyrrolonyl, oxazolonyl, imidazolonyl, cyclopentenonyl, alkyl, substituted alkyl, aryl, substituted aryl, heterocycle, or substituted heterocycle;
R4 is hydrogen, halomethyl, hydroxymethyl, diazomethyl, acyloxymethyl, ketone, ketoamide, ketoacid, ketoester, alkyl, substituted alkyl aryl, substituted aryl, heterocycle, or substituted heterocycle; and
the bond indicated by the asterisk is a single or double bond.

10. A compound having the formula: 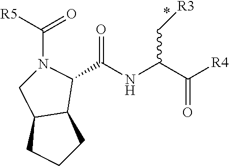
wherein:
R5 is pyrazole, substituted pyrazole, imidazole, substituted imidazole, piperidine, substituted piperidine, pyridine, substituted pyridine, pyridone, substituted pyridone, indole, methoxyindole, other substituted indole, isoindole, substituted isoindole, purine, substituted purine, benzyloxy, substituted benzyloxy, benzylamino, substituted benzylamino, phenoxymethyl, substituted phenoxymethyl, phenylaminomethyl, substituted phenylaminomethyl, phenylethyl, substituted phenylethyl, alkyl, substituted alkyl, aryl, substituted aryl, heterocycle, or substituted heterocycle;
R3 is gamma-lactamyl, cyclopentanone, cyclopentyl, beta-lactamyl, cyclobutanonyl, cyclobutyl, acetonyl, acetyl, gamma-lactonyl, furanonyl, pyrrolonyl, oxazolonyl, imidazolonyl, cyclopentenonyl, alkyl, substituted alkyl, aryl, substituted aryl, heterocycle, or substituted heterocycle;
R4 is hydrogen, halomethyl, hydroxymethyl, diazomethyl, acyloxymethyl, ketone, ketoamide, ketoacid, ketoester, alkyl, substituted alkyl aryl, substituted aryl, heterocycle, or substituted heterocycle; and
the bond indicated by the asterisk is a single or double bond.

11. A compound having the formula: 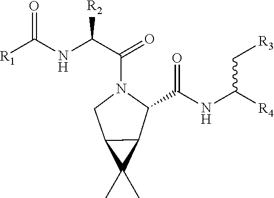
wherein:
R₁ and R₂ are independently alkyl, cycloalkyl, substituted alkyl, heteroalkyl, or heterocycloalkyl;
R₃ is gamma-lactamyl, cyclopentanone, cyclopentyl, beta-lactamyl, cyclobutanonyl, cyclobutyl, acetonyl, acetyl, gamma-lactonyl, furanonyl, pyrrolonyl, oxazolonyl, imidazolonyl, cyclopentenonyl, alkyl, substituted alkyl, aryl, substituted aryl, heterocycle, or substituted heterocycle; and
R₄ is primary alpha-ketoamide, secondary alpha-ketoamide, or tertiary alpha-ketoamide.

12. A compound having the formula: 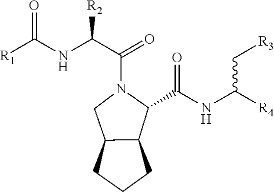
wherein:
R₁ and R₂ are independently alkyl, cycloalkyl, substituted alkyl, heteroalkyl, or heterocycloalkyl;
R₃ is gamma-lactamyl, cyclopentanone, cyclopentyl, beta-lactamyl, cyclobutanonyl, cyclobutyl, acetonyl, acetyl, gamma-lactonyl, furanonyl, pyrrolonyl, oxazolonyl, imidazolonyl, cyclopentenonyl, alkyl, substituted alkyl, aryl, substituted aryl, heterocycle, or substituted heterocycle; and
R₄ is primary alpha-ketoamide, secondary alpha-ketoamide, or tertiary alpha-ketoamide.

13. A compound having the formula: 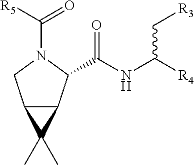
wherein:
R₃ is gamma-lactamyl, cyclopentanone, cyclopentyl, beta-lactamyl, cyclobutanonyl, cyclobutyl, acetonyl, acetyl, gamma-lactonyl, furanonyl, pyrrolonyl, oxazolonyl, imidazolonyl, cyclopentenonyl, alkyl, substituted alkyl, aryl, substituted aryl, heterocycle, or substituted heterocycle;
R₄ is primary alpha-ketoamide, secondary alpha-ketoamide, tertiary alpha-ketoamide, or nitrile; and
R₅ is pyrazole, substituted pyrazole, imidazole, substituted imidazole, piperidine, substituted piperidine, pyridine, substituted pyridine, pyridone, substituted pyridone, indole, methoxyindole, other substituted indole, isoindole, substituted isoindole, purine, substituted purine, benzyloxy, substituted benzyloxy, benzylamino, substituted benzylamino, phenoxymethyl, substituted phenoxymethyl, phenylaminomethyl, substituted phenylaminomethyl, phenylethyl, substituted phenylethyl, phenylmethyl, substituted phenylmethyl, alkyl, substituted alkyl, aryl, substituted aryl, heterocycle, or substituted heterocycle.

14. A compound having the formula: 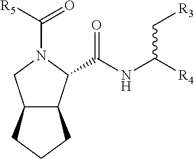
wherein:
R₃ is gamma-lactamyl, cyclopentanone, cyclopentyl, beta-lactamyl, cyclobutanonyl, cyclobutyl, acetonyl, acetyl, gamma-lactonyl, furanonyl, pyrrolonyl, oxazolonyl, imidazolonyl, cyclopentenonyl, alkyl, substituted alkyl, aryl, substituted aryl, heterocycle, or substituted heterocycle;
R₄ is primary alpha-ketoamide, secondary alpha-ketoamide, tertiary alpha-ketoamide, or nitrile; and
R₅ is pyrazole, substituted pyrazole, imidazole, substituted imidazole, piperidine, substituted piperidine, pyridine, substituted pyridine, pyridone, substituted pyridone, indole, methoxyindole, other substituted indole, isoindole, substituted isoindole, purine, substituted purine, benzyloxy, substituted benzyloxy, benzylamino, substituted benzylamino, phenoxymethyl, substituted phenoxymethyl, phenylaminomethyl, substituted phenylaminomethyl, phenylethyl, substituted phenylethyl, phenylmethyl, substituted phenylmethyl, alkyl, substituted alkyl, aryl, substituted aryl, heterocycle, or substituted heterocycle.

15. A compound having the formula: 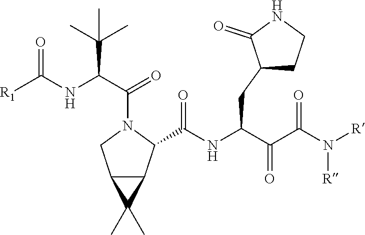
wherein:
R₁ is alkyl, cycloalkyl, substituted alkyl, heteroalkyl, or heterocycloalkyl; and
R′ and R″ are independently H, alkyl, aryl, heteroalkyl, alkenyl, alkynyl, heteroaryl, cycloalkyl, heterocyclyl, arylalkyl, heteroarylalkyl, or substituted alkyl,
optionally wherein R′ and R″ are interconnected.

16. The compound of claim 15, wherein R₁ is bicyclo[1.1.1]pentanyl.

17. The compounds of claim 15, wherein the compound is:

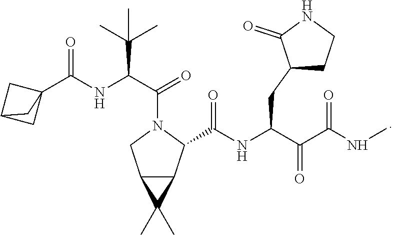 [SMILES: CNC(=O)C(=O)[C@H](C[C@@H]1CCNC1=O)NC(=O)[C@@H]1[C@@H]2[C@H](CN1C(=O)[C@@H](NC(=O)C13CC(C1)C3)C(C)(C)C)C2(C)C]

18. The compounds of claim 15, wherein the compound is:

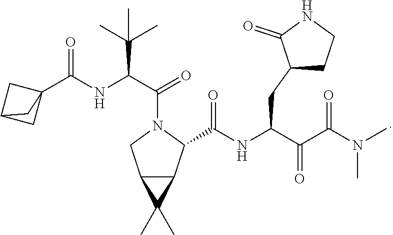 [SMILES: CN(C)C(=O)C(=O)[C@H](C[C@@H]1CCNC1=O)NC(=O)[C@@H]1[C@@H]2[C@H](CN1C(=O)[C@@H](NC(=O)C13CC(C1)C3)C(C)(C)C)C2(C)C]

19. The compound of claim 15, wherein R₁ is neopentanyl.

20. The compound of claim 15, wherein the compound is:

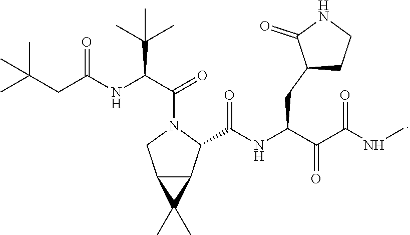 [SMILES: CNC(=O)C(=O)[C@H](C[C@@H]1CCNC1=O)NC(=O)[C@@H]1[C@@H]2[C@H](CN1C(=O)[C@@H](NC(=O)CC(C)(C)C)C(C)(C)C)C2(C)C]

21. The compound of claim 15, wherein the compound is:

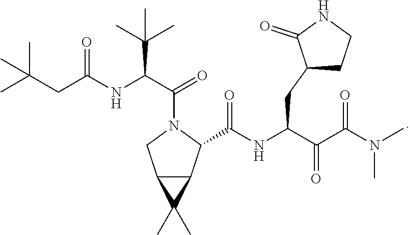 [SMILES: CN(C)C(=O)C(=O)[C@H](C[C@@H]1CCNC1=O)NC(=O)[C@@H]1[C@@H]2[C@H](CN1C(=O)[C@@H](NC(=O)CC(C)(C)C)C(C)(C)C)C2(C)C]

22. The compound of claim 15, wherein R′ and R″ are each methyl.

23. The compound of claim 15, wherein the compound is:

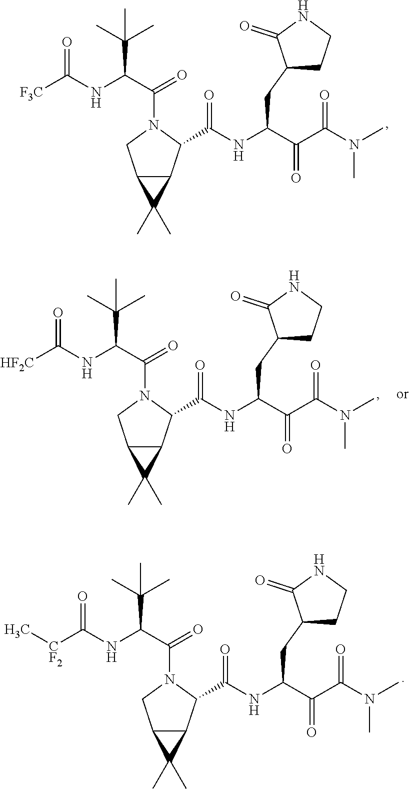

24. The compound of claim 15, wherein the compound is:

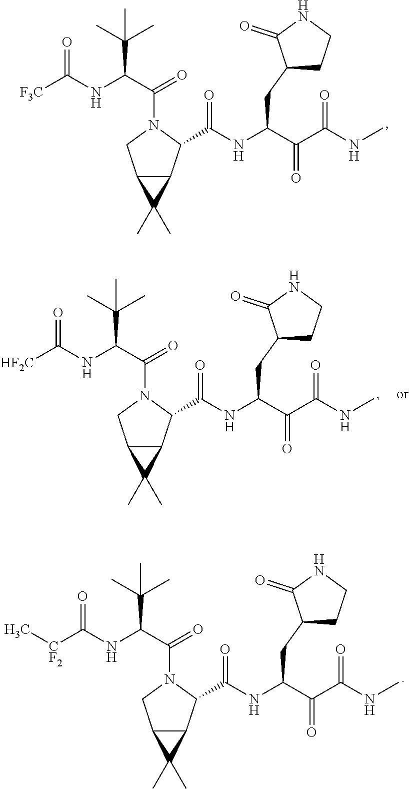

25. A compound having the formula: 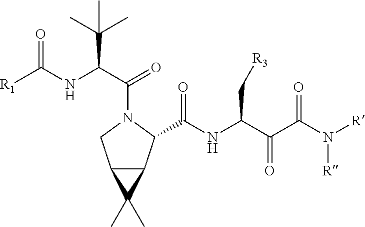
wherein:
R₁ is alkyl, cycloalkyl, substituted alkyl, heteroalkyl, or heterocycloalkyl;
R₃ is pyridinyl, substituted pyridinyl, phenyl, substituted phenyl, heteroaryl, or heterocyclyl; and
R′ and R″ are independently H, alkyl, aryl, heteroalkyl, alkenyl, alkynyl, heteroaryl, cycloalkyl, heterocyclyl, arylalkyl, heteroarylalkyl, or substituted alkyl,
optionally wherein R′ and R″ are interconnected.

26. The compound of claim 25, wherein R₁ is neopentanyl, R₃ is pyridinyl, and R′ and R″ are independently H or methyl.

27. The compound of claim 25, wherein the compound is:

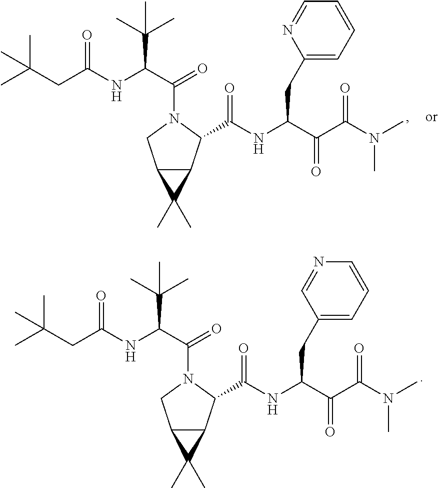

28. The compound of claim 25, wherein the compound is:

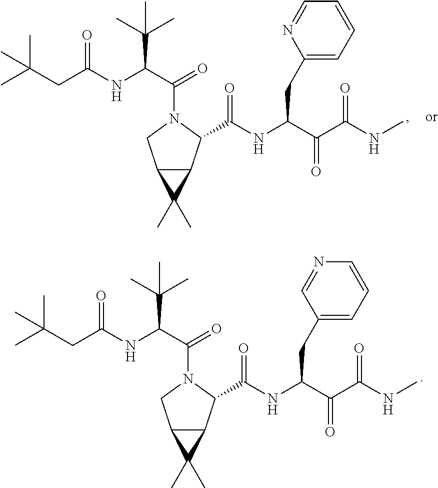

29. The compound of claim 25, wherein R₁ is bicyclo[1.1.1]pentanyl, R₃ is pyridinyl, and R′ and R″ are independently H or methyl.

30. The compound of claim 25, wherein the compound is:

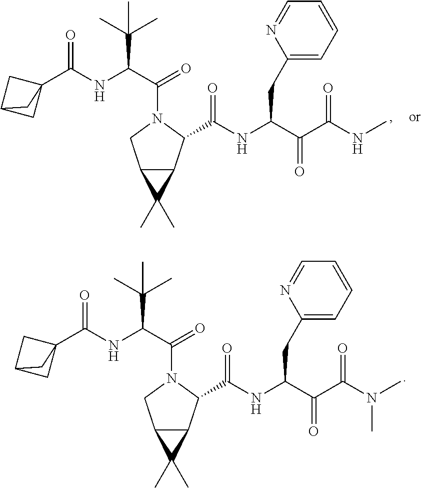

31. The compound of claim 25, wherein the compound is:

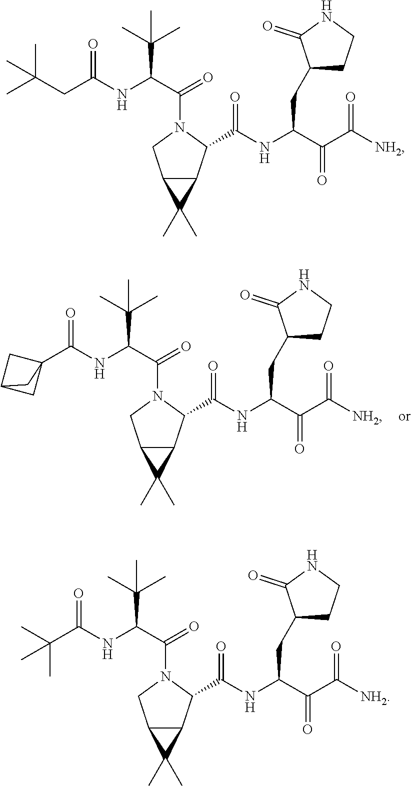 [SMILES: CC(C)(C)CC(=O)N[C@H](C(=O)N1C[C@H]2[C@@H]([C@H]1C(=O)N[C@@H](C[C@@H]1CCNC1=O)C(=O)C(N)=O)C2(C)C)C(C)(C)C ; CC(C)(C)[C@H](NC(=O)C12CC(C1)C2)C(=O)N1C[C@H]2[C@@H]([C@H]1C(=O)N[C@@H](C[C@@H]1CCNC1=O)C(=O)C(N)=O)C2(C)C ; "or" ; CC(C)(C)C(=O)N[C@H](C(=O)N1C[C@H]2[C@@H]([C@H]1C(=O)N[C@@H](C[C@@H]1CCNC1=O)C(=O)C(N)=O)C2(C)C)C(C)(C)C]

32. A compound having the formula: 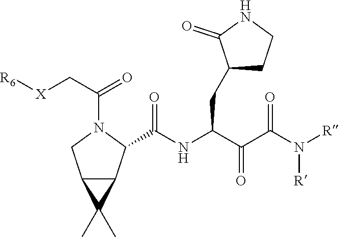
wherein:
X is CH₂, NH, or O;
R₆ is aryl, heteroaryl, substituted aryl, or substituted heteroaryl; and
R′ and R″ are independently H, alkyl, aryl, heteroalkyl, alkenyl, alkynyl, heteroaryl, cycloalkyl, heterocyclyl, arylalkyl, heteroarylalkyl, or substituted alkyl,
optionally wherein R′ and R″ are interconnected.

33. The compound of claim 32, wherein X is CH₂ and R₆ is substituted phenyl.

34. The compound of claim 32, wherein the compound is:

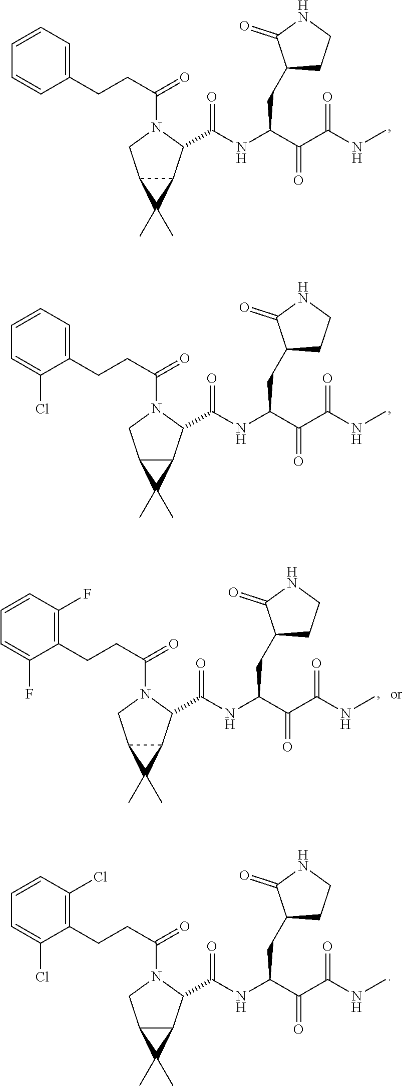 [SMILES: CNC(=O)C(=O)[C@H](C[C@@H]1CCNC1=O)NC(=O)[C@@H]1[C@@H]2[C@H](CN1C(=O)CCc1ccccc1)C2(C)C ; CNC(=O)C(=O)[C@H](C[C@@H]1CCNC1=O)NC(=O)[C@@H]1[C@@H]2[C@H](CN1C(=O)CCc1ccccc1Cl)C2(C)C ; CNC(=O)C(=O)[C@H](C[C@@H]1CCNC1=O)NC(=O)[C@@H]1[C@@H]2[C@H](CN1C(=O)CCc1c(F)cccc1F)C2(C)C ; CNC(=O)C(=O)[C@H](C[C@@H]1CCNC1=O)NC(=O)[C@@H]1[C@@H]2[C@H](CN1C(=O)CCc1c(Cl)cccc1Cl)C2(C)C]

35. The compound of claim 32, wherein X is NH and R₆ is substituted phenyl.

36. The compound of claim 35, wherein the compound is:

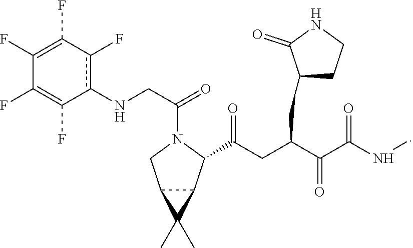

37. The compound of claim 35, wherein R₆ is trihalophenyl.

38. The compound of claim 37, wherein the compound is:

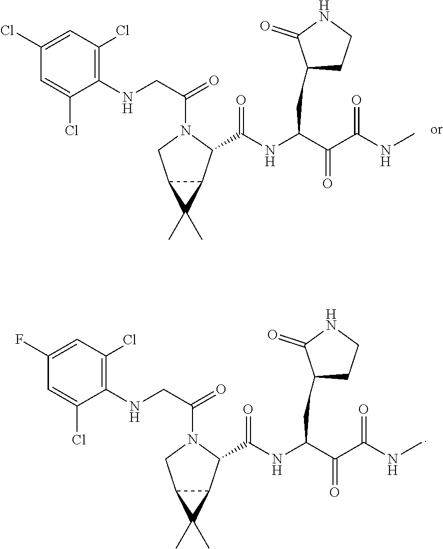 [SMILES: CNC(=O)C(=O)[C@H](C[C@@H]1CCNC1=O)NC(=O)[C@@H]1[C@@H]2[C@H](CN1C(=O)CNc1c(Cl)cc(Cl)cc1Cl)C2(C)C ; CNC(=O)C(=O)[C@H](C[C@@H]1CCNC1=O)NC(=O)[C@@H]1[C@@H]2[C@H](CN1C(=O)CNc1c(Cl)cc(F)cc1Cl)C2(C)C]

39. The compound of claim 35, wherein R₆ is trifluorophenyl.

40. The compound of claim 39, wherein the compound is:

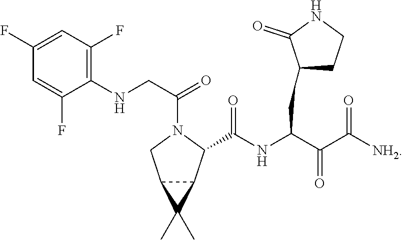 [SMILES: CC1(C)[C@@H]2[C@@H](C(=O)N[C@@H](C[C@@H]3CCNC3=O)C(=O)C(N)=O)N(C(=O)CNc3c(F)cc(F)cc3F)C[C@@H]21]

41. The compound of claim 39, wherein the compound is:

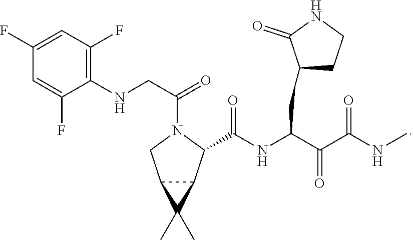 [SMILES: CNC(=O)C(=O)[C@H](C[C@@H]1CCNC1=O)NC(=O)[C@@H]1[C@@H]2[C@H](CN1C(=O)CNc1c(F)cc(F)cc1F)C2(C)C]

42. The compound of claim 39, wherein the compound is:

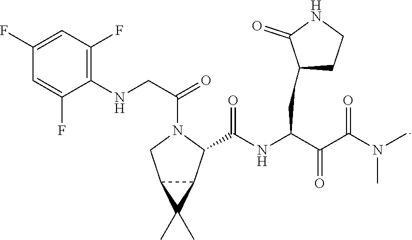 [SMILES: CN(C)C(=O)C(=O)[C@H](C[C@@H]1CCNC1=O)NC(=O)[C@@H]1[C@@H]2[C@H](CN1C(=O)CNc1c(F)cc(F)cc1F)C2(C)C]

43. The compound of claim 32, wherein X is O and R₆ is substituted phenyl.

44. The compound of claim 43, wherein R₆ is trihalophenyl.

45. The compound of claim 44, wherein the compound is:

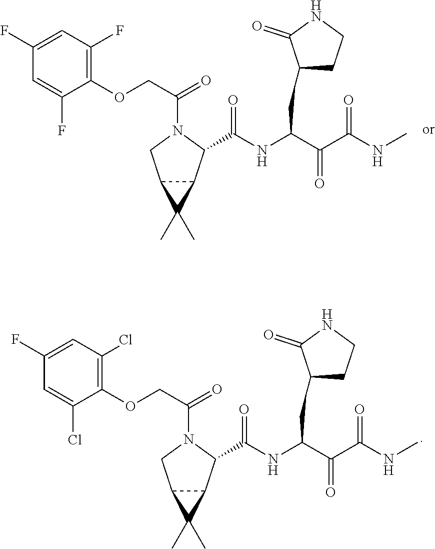 [SMILES: CNC(=O)C(=O)[C@H](C[C@@H]1CCNC1=O)NC(=O)[C@@H]1[C@@H]2[C@H](CN1C(=O)COc1c(F)cc(F)cc1F)C2(C)C ; CNC(=O)C(=O)[C@H](C[C@@H]1CCNC1=O)NC(=O)[C@@H]1[C@@H]2[C@H](CN1C(=O)COc1c(Cl)cc(F)cc1Cl)C2(C)C]

46. A compound having the formula: 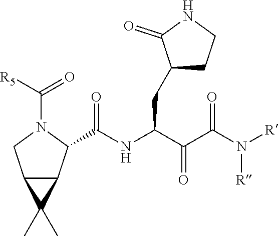
wherein:
R₅ is alkyl, substituted alkyl, aryl, heteroalkyl, heteroaryl, cycloalkyl, heterocyclyl, arylalkyl, cyclylalkyl, heterocyclylalkyl, arylheteroalkyl, heteroarylalkyl, substituted arylalkyl, substituted heteroaryl, or substituted heteroarylalkyl; and
R′ and R″ are independently H, alkyl, aryl, heteroalkyl, alkenyl, alkynyl, heteroaryl, cycloalkyl, heterocyclyl, arylalkyl, heteroarylalkyl, or substituted alkyl,
optionally wherein R′ and R″ are interconnected.

47. The compound of claim 46, wherein the compound is:

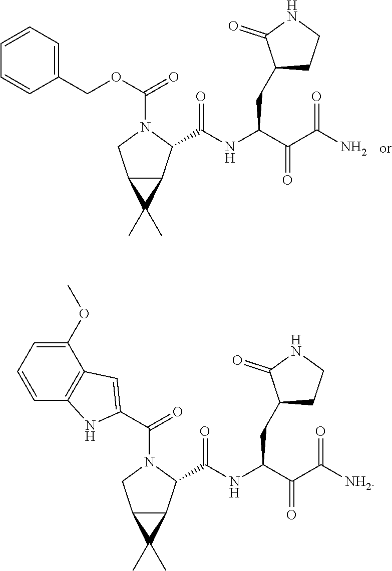 [SMILES: CC1(C)[C@@H]2[C@@H](C(=O)N[C@@H](C[C@@H]3CCNC3=O)C(=O)C(N)=O)N(C(=O)OCc3ccccc3)C[C@@H]21 ; COc1cccc2[nH]c(C(=O)N3C[C@H]4[C@@H]([C@H]3C(=O)N[C@@H](C[C@@H]3CCNC3=O)C(=O)C(N)=O)C4(C)C)cc12]

48. The compound of claim 46, wherein R′ is methyl and R″ is H or methyl.

49. A compound having the formula: 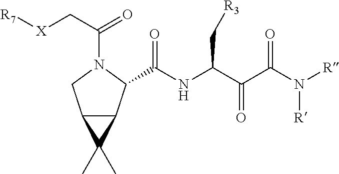
wherein:
X is CH₂, NH, or O;
R₃ is pyridinyl, substituted pyridinyl, phenyl, substituted phenyl, heteroaryl, or heterocyclyl;
R₇ is a substituted phenyl; and
R′ and R″ are independently H, alkyl, aryl, heteroalkyl, alkenyl, alkynyl, heteroaryl, cycloalkyl, heterocyclyl, arylalkyl, heteroarylalkyl, or substituted alkyl,
optionally wherein R′ and R″ are interconnected.

50. The compound of claim 49, wherein X is NH and R₇ is a trihalophenyl.

51. The compound of claim 50, wherein R₇ is a trifluorophenyl.

52. The compound of claim 51, wherein R₃ is pyridinyl.

53. The compound of claim 52, wherein the compound is:

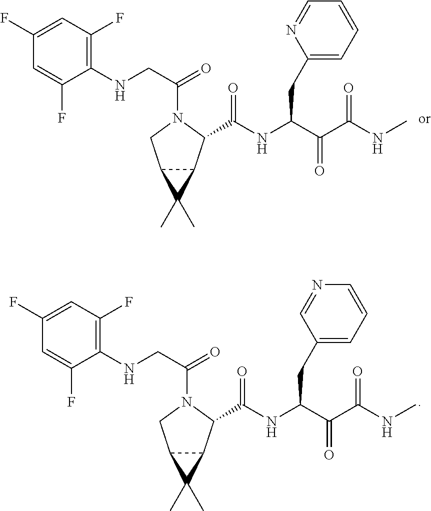 [SMILES: CNC(=O)C(=O)[C@H](Cc1ccccn1)NC(=O)[C@@H]1[C@@H]2[C@H](CN1C(=O)CNc1c(F)cc(F)cc1F)C2(C)C ; CNC(=O)C(=O)[C@H](Cc1cccnc1)NC(=O)[C@@H]1[C@@H]2[C@H](CN1C(=O)CNc1c(F)cc(F)cc1F)C2(C)C]

54. A compound having the formula: 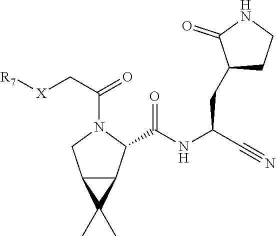
wherein:
X is CH₂, NH, or O; and
R₇ is a substituted phenyl.

55. The compound of claim 54, wherein the compound is:

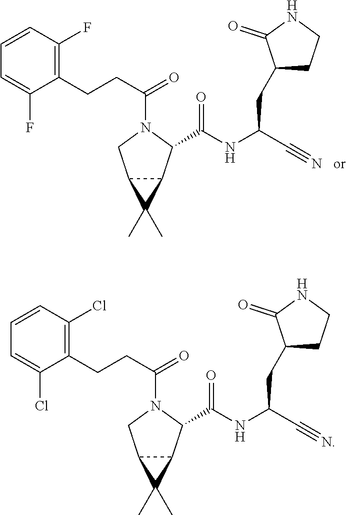 [SMILES: CC1(C)[C@@H]2[C@@H](C(=O)N[C@H](C#N)C[C@@H]3CCNC3=O)N(C(=O)CCc3c(F)cccc3F)C[C@@H]21 ; CC1(C)[C@@H]2[C@@H](C(=O)N[C@H](C#N)C[C@@H]3CCNC3=O)N(C(=O)CCc3c(Cl)cccc3Cl)C[C@@H]21]

56. The compound of claim 54, wherein X is NH and R₇ is a trihalophenyl.

57. The compound of claim 56, wherein the compound is:

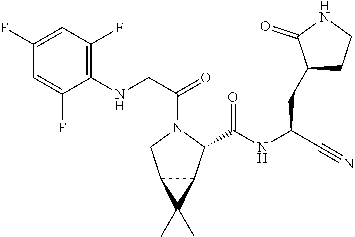 [SMILES: CC1(C)[C@@H]2[C@@H](C(=O)N[C@H](C#N)C[C@@H]3CCNC3=O)N(C(=O)CNc3c(F)cc(F)cc3F)C[C@@H]21]

58. The compound of claim 54, wherein X is O and R₇ is a trihalophenyl.

59. The compound of claim 56, wherein the compound is:

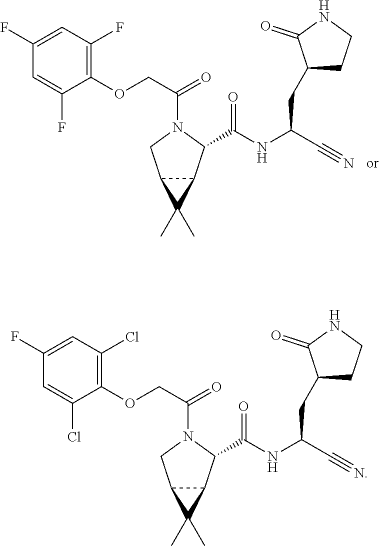 [SMILES: CC1(C)[C@@H]2[C@@H](C(=O)N[C@H](C#N)C[C@@H]3CCNC3=O)N(C(=O)COc3c(F)cc(F)cc3F)C[C@@H]21 ; CC1(C)[C@@H]2[C@@H](C(=O)N[C@H](C#N)C[C@@H]3CCNC3=O)N(C(=O)COc3c(Cl)cc(F)cc3Cl)C[C@@H]21]

60. A pharmaceutical composition, comprising:
the compound of any one of claims 1 to 59; and
a pharmaceutically acceptable carrier.

61. The pharmaceutical composition of claim 60, wherein the pharmaceutical composition is formulated for parenteral, inhalational, intranasal, subcutaneous, intramuscular, or intravenous administration.

62. The pharmaceutical composition of claim 60 or claim 61, further comprising a coronavirus polymerase inhibitor.

63. The pharmaceutical composition of claim 62, wherein the coronavirus polymerase inhibitor is a SARS-CoV-2 polymerase inhibitor.

64. The pharmaceutical composition of claim 62 or claim 63, wherein the coronavirus polymerase inhibitor is selected from remdesivir and favipiravir.

65. A method of treating or preventing a coronavirus infection in an individual, comprising:
administering to an individual the pharmaceutical composition of any one of claims 60 to 64 in an amount effective to treat or prevent a coronavirus infection in the individual.

66. The method according to claim 65, wherein the method is for treating or preventing a SARS-CoV-2 infection in the individual.

67. A method of treating or preventing a SARS-CoV-2 infection in an individual, comprising:
administering to an individual a pharmaceutical composition comprising boceprevir (BPV), narlaprevir (NPV), telaprevir (TPV), rupintrivir, or any combination thereof, in an amount effective to treat or prevent a SARS-CoV-2 infection in the individual.

68. The method according to any one of claims 65 to 67, further comprising administering an effective amount of a coronavirus polymerase inhibitor to the individual.

69. The method according to claim 68, wherein the coronavirus polymerase inhibitor is a SARS-CoV-2 polymerase inhibitor.

70. The method according to claim 68 or claim 69, wherein the coronavirus polymerase inhibitor is present in the same pharmaceutical composition as the compound.

71. The method according to any one of claims 68 to 70, wherein the coronavirus polymerase inhibitor is selected from remdesivir and favipiravir.

72. A kit comprising:
the pharmaceutical composition of any one of claims 60 to 64; and
instructions for administering an effective amount of the pharmaceutical composition to an individual to treat or prevent a coronavirus infection in the individual.

73. The kit of claim 72, wherein the pharmaceutical composition is present in two or more unit dosages.

74. The kit of claim 72 or claim 73, further comprising a coronavirus polymerase inhibitor.

75. The kit of claim 74, wherein the coronavirus polymerase inhibitor is selected from remdesivir and favipiravir.

76. A method of assessing inhibition of coronavirus protease activity by an agent, comprising:
culturing a cell comprising a first nucleic acid sequence that encodes a coronavirus main protease (Mpro) and a second nucleic acid sequence that encodes a fusion protein comprising a substrate for the Mpro disposed between an optically detectable protein and a membrane localization signal, under conditions in which the Mpro and fusion protein are expressed and the fusion protein is localized to the cell membrane via the membrane localization signal;
introducing an agent into the cell; and
assessing cellular localization of the optically detectable protein, wherein retention of cell membrane localization of the optically detectable protein indicates that the agent is an inhibitor of the Mpro.

77. The method according to claim 76, wherein the Mpro is SARS-CoV-2 Mpro.

78. The method according to claim 76 or claim 77, wherein the substrate for the Mpro comprises the sequence SAVLQ↓SGFRK (SEQ ID NO:1).

79. The method according to any one of claims 76 to 78, wherein the optically detectable protein is a fluorescent protein.

80. The method according to claim 79, wherein the fluorescent protein is a green fluorescent protein (GFP), a blue fluorescent protein (BFP), a cyan fluorescent protein (CFP), a yellow fluorescent protein (YFP), an orange fluorescent protein (OFP), or a red fluorescent protein (RFP).

81. The method according to any one of claims 76 to 78, wherein the optically detectable protein is a luminescent protein.

82. The method according to claim 81, wherein the luminescent protein is a luciferase.

83. The method according to any one of claims 76 to 82, wherein the cell is a human cell.

84. A method of assessing inhibition of activity of a protease by an agent, comprising:
culturing a cell comprising a nucleic acid sequence that encodes a reporter polypeptide comprising in order:
a first portion of a split reporter protein;
a first flexible linker comprising a substrate for a protease;
the protease;
a second flexible linker comprising a substrate for the protease; and
a remaining portion of the split reporter protein,
under conditions in which the cell expresses the reporter polypeptide and the protease cleaves one or both of the flexible linkers in the absence of inhibition of activity of the protease, thereby inactivating the split reporter protein by separating the first and remaining portions thereof;
introducing an agent into the cell; and
assaying for activity of the reporter protein to assess inhibition of activity of the protease by the agent, wherein activity of the reporter protein indicates inhibition of activity of the protease by the agent.

85. The method according to claim 84, wherein the protease is a coronavirus main protease (Mpro).

86. The method according to claim 85, wherein the Mpro is SARS-CoV-2 Mpro.

87. The method according to claim 86, wherein the first flexible linker and the second flexible linker comprise a substrate for the SARS-CoV-2 Mpro independently selected from: a substrate comprising the amino acid sequence TSAVLQ↓SGFRK (SEQ ID NO:2) and a substrate comprising the amino acid sequence VTFQ↓SAVKRTIKGTTS (SEQ ID NO:3).

88. The method according to any one of claims 84 to 87, wherein the split reporter protein is a split luminescent protein.

89. The method according to claim 88, wherein the split luminescent protein is a split luciferase.

90. The method according to any one of claims 84 to 89, wherein the cell is a human cell.

91. The method according to any one of claims 84 to 90, wherein the agent is a small molecule.


SEQUENCE LISTING (6 sequences)

SEQ ID NO: 1 - protein, 10 aa, Artificial sequence
  synthetic sequence
SAVLQSGFRK

SEQ ID NO: 2 - protein, 11 aa, Artificial sequence
  synthetic sequence
TSAVLQSGFRK

SEQ ID NO: 3 - protein, 16 aa, Artificial sequence
  synthetic sequence
VTFQSAVKRTIKGTTS

SEQ ID NO: 4 - protein, 15 aa, Artificial sequence
  synthetic sequence
  SITE (1)..(1): The amino acid at position 1 comprises TF5
  SITE (14)..(15): The amino acids at positions 14 and 15 comprise a TQ5 quencher
KTSAVLQSGFRKMEM

SEQ ID NO: 5 - protein, 523 aa, Artificial sequence
  synthetic sequence
MVTGYRLFEEILAGGGGSGMTSAVLQSGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQSAVKRTIKGTTSGGGSGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGSYPYDVPDYA

SEQ ID NO: 6 - DNA, 1569 bp, Artificial sequence
  synthetic sequence
atggtgaccggctaccggctgttcgaggagattctggcgggtggcggaggatctggaatgacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacaactagtggcggaggctctggaggagtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcggaggtagtggtggtagctacccttacgatgtccctgactacgcg